(19) United States
(12) Patent Application Publication
(10) Pub. No.: US 2005/0064577 A1
(43) Pub. Date: Mar. 24, 2005

(54) Hydrogen production with photosynthetic organisms and from biomass derived therefrom

(76) Inventor: Isaac Berzin, Newton, MA (US)

Correspondence Address: Michael J. Pomianek, Ph.D.;Wolf, Greenfield & Sacks, P.C., 600 Atlantic Avenue, Boston, MA, 02210-2206, US

(21) Appl. No.: 10/924,742

(22) Filed: Aug. 23, 2004

Related U.S. Application Data
(63) Continuation-in-part of application No. PCT/US03/15364, filed on May 13, 2003.
(60) Provisional application No. 60/380,179, filed on May 13, 2002.
(60) Provisional application No. 60/497,445, filed on Aug. 22, 2003.

(51) Int. Cl. A61L 9/01

(52) U.S. Cl. 435/266

(57) ABSTRACT

Certain embodiments and aspects of the present invention relate to photobioreactor apparatus designed to contain a liquid medium comprising at least one species of photosynthetic organism therein, and to methods of using the photobioreactor apparatus as part of a hydrogen production process and system configured to generate hydrogen with and/or from biomass produced in the photobioreactor apparatus. In certain embodiments, the disclosed hydrogen production systems and methods, photobioreactor apparatus, methods of using such apparatus, and/or gas treatment systems and methods provided herein can be utilized as part of an integrated combustion and hydrogen production method and system, wherein photosynthetic organisms utilized within the photobioreactor are used to at least partially remove certain pollutant compounds contained within combustion gases, e.g. CO₂ and/or NOₓ, and are subsequently harvested from the photobioreactor, processed, and utilized as a fuel source for generating hydrogen and/or as a fuel source for a combustion device (e.g. an electric power plant generator and/or incinerator).

### RELATED APPLICATIONS

This application is a continuation-in-part of PCT International Application No. PCT/US03/15364 filed May 13, 2003, which was published under PCT Article 21(2) in English, and claims the benefit of priority via PCT/US03/15364 under Title 35, U.S.C. §119(e) to U.S. provisional application Ser. No. 60/380,179, filed May 13, 2002. Both applications are incorporated herein by reference.

This non-provisional application claims the benefit under Title 35, U.S.C. §119(e) of co-pending U.S. provisional application Ser. No. 60/497,445, filed, Aug. 22, 2003, which is incorporated herein by reference.

### FIELD OF INVENTION

The invention relates generally to hydrogen production using photosynthetic organisms and/or from biomass derived therefrom, and in certain embodiments, from biomass produced by photobioreactors operated for the treatment of gases, such as flue gases.

### BACKGROUND OF THE INVENTION

In the United States alone, there are 400 coal burning power plants representing 1,600 generating units and another 10,000 fossil fuel plants. Although coal plants are the dirtiest of the fossil fuel users, oil and gas plants also produce flue gas (combustion gases) that may include CO₂, NO_{X}, SO_{X}, mercury, mercury-containing compounds, particulates and other pollutant materials.

Photosynthesis is the carbon recycling mechanism of the biosphere. In this process, photosynthetic organisms, such as plants, synthesize carbohydrates and other cellular materials by CO₂ fixation. One of the most efficient converters of CO₂ and solar energy to biomass are algae, the fastest growing plants on earth and one of nature's simplest microorganisms. In fact, over 90% of CO₂ fed to algae can be absorbed, mostly in the production of cell mass. (Sheehan John, Dunahay Terri, Benemann John R., Roessler Paul, “A Look Back at the U.S. Department of Energy's Aquatic Species Program: Biodiesel from Algae,” 1998, NERL/TP-580-24190; hereinafter “Sheehan et al. 1998”). In addition, algae are capable of growing in saline waters that are unsuitable for agriculture.

Using algal biotechnology, CO₂ bio-regeneration can be advantageous due to the production of a useful, high-value products from waste CO₂. Production of algal biomass during combustion gas treatment for CO₂ reduction is an attractive concept since dry algae has a heating value roughly equivalent to coal. Algal biomass can also be turned into high quality liquid fuel (similar to crude oil) through thermochemical conversion by known technologies. Algal biomass can also be used for gasification to produce highly flammable organic fuel gases, suitable for use in gas-burning power plants. (e.g., see Reed T. B. and Gaur S. “A Survey of Biomass Gasification” NREL, 2001; hereinafter “Reed and Gaur 2001”).

Approximately 114 kilocalories (477 kJ) of free energy are stored in plant biomass for every mole of CO₂ fixed during photosynthesis. Algae are responsible for about one-third of the net photosynthetic activity worldwide. Photosynthesis can be simply represented by the equation:
CO₂+H₂O+light→(CH₂O)+O₂
where (CH₂O) represents a generalized chemical formula for carbonaceous biomass.

Although photosynthesis is fundamental to the conversion of solar radiation into stored biomass, efficiencies can be limited by the limited wavelength range of light energy capable of driving photosynthesis (400-700 nm, which is only about half of the total solar energy). Other factors, such as respiration requirements (during dark periods), efficiency of absorbing sunlight and other growth conditions can affect photosynthetic efficiencies in algal bioreactors. The net result is an overall photosynthetic efficiency that can range from 6% in the field (for open pond-type reactors) to 24% in the most efficient lab scale photobioreactors.

Algal cultures can also be used for biological NO_{X} removal from combustion gases. (Nagase Hiroyasu, Ken-Ichi Yoshihara, Kaoru Eguchi, Yoshiko Yokota, Rie Matsui, Kazumasa Hirata and Kazuhisa Miyamoto, “Characteristics of Biological NO_{X} Removal from Flue Gas in a Dunaliella tertiolecta Culture System,” Journal of Fermentation and Bioengineering, 83, 1997; hereinafter “Hiroyasu et al. 1997”). Some algae species can remove NO_{X} at a wide range of NO_{X} concentrations and combustion gas flow rates. Nitrous oxide (NO), a major NO_{X} component, is dissolved in the aqueous phase, after which it is oxidized to NO₂ and assimilated by the algal cell. The following equation describes the reaction of dissolved NO with dissolved O₂:
4NO+O₂+2H₂O→4NO₂−+4H⁺

The dissolved NO₂ is then used by the algal as a nitrogen source and is partially converted into gaseous N₂. The dissolution of NO in the aqueous phase is believed to be the rate-limiting step in this NO_{X} removal process. This process can be described by the following equation, when k is a temperature-dependent rate constant:
−d[NO]/dt=4k[NO]²[O₂]

For example, NO_{X} removal using the algae species Dunaliella can occur under both light and dark conditions, with an efficiency of NOₓ, removal of over 96% (under light conditions).

Creating fuels from algal biotechnology has also been proposed. Over an 18-year period, the U.S. Department of Energy (DOE) funded an extensive series of studies to develop renewable transportation fuels from algae (Sheehan et al. 1998). In Japan, government organizations (MITI), in conjunction with private companies, have invested over $250 million into algal biotechnology. Each program took a different approach but because of various problems, addressed by certain embodiments of the present invention, none has been commercially successful to date.

A major obstacle for feasible algal bio-regeneration and pollution abatement has been an efficient, yet cost-effective, growth system. DOE's research focused on growing algae in massive open ponds as big as 4 km². The ponds require low capital input; however, algae grown in open and uncontrolled environments result in low algal productivity. The open pond technology made growing and harvesting the algae prohibitively expensive, since massive amounts of dilute algal waters required very large agitators, pumps and centrifuges. Furthermore, with low algal productivity and large flatland requirements, this approach could, in the best-case scenario, be applicable to only 1% of U.S. power plants. (Sheehan et al. 1998). On the other hand, the MITI approach, with stricter land constraints, focused on very expensive closed algal photobioreactors utilizing fiber optics for light transmission. In these controlled environments, much higher algal productivity was achieved, but the algal growth rates were not high enough to offset the capital costs of the expensive systems utilized.

Typical conventional photobioreactors have taken several forms, such as cylindrical or tubular bioreactors, for example as taught by Yogev et al. in U.S. Pat. No. 5,958,761. These bioreactors, when oriented horizontally, typically require additional energy to provide mixing (e.g., pumps), thus adding significant capital and operational expense. In this orientation, the O₂ produced by photosynthesis can become trapped in the system, thus causing a reduction in algal proliferation. Other known photobioreactors are oriented vertically and agitated pneumatically. Many such photobioreactors operate as “bubble columns,” as discussed below. Some known photobioreactor designs rely on artificial lighting, e.g. fluorescent lamps, (such as described by Kodo et al. in U.S. Pat. No. 6,083,740). Photobioreactors that do not utilize solar energy but instead rely solely on artificial light sources can require enormous energy input.

Many conventional photobioreactors comprise cylindrical algal photobioreactors that can be categorized as either “bubble columns” or “air lift reactors.” Bubble columns are typically translucent large diameter containers filled with algae suspended in liquid medium, in which gases are bubbled at the bottom of the container. Since no precisely defined flow lines are reproducibly formed, it can be difficult to control the mixing properties of the system which can lead to low mass transfer coefficients poor photomodulation, and low productivity. Air lift reactors typically consist of vertically oriented concentric tubular containers, in which the gases are bubbled at the bottom of the inner tube. The pressure gradient created at the bottom of this tube creates an annular liquid flow (upwards through the inner tube and downwards between the tubes). The external tube is made out of translucent material, while the inner tube is usually opaque. Therefore, the algae are exposed to light while passing between the tubes, and to darkness while passing in the inner tube. The light-dark cycle is determined by the geometrical design of the reactor (height, tube diameters) and by operational parameters (e.g., gas flow rate). Air lift reactors can have higher mass transfer coefficients and algal productivity when compared to bubble columns. However, control over the flow patterns within an air lift reactor to achieve a desired level of mixing and photomodulation can still be difficult or impractical. In addition, because of geometric design constraints, during large-scale, outdoor algal production, both types of cylindrical-photobioreactors can suffer from low productivity, due to factors related to light reflection and auto-shading effects (in which one column is shading the other).

A fuel of increasing importance and substantial short- and long-term future significance is hydrogen. The value of hydrogen in producing clean, abundant energy cannot be overestimated. Rapidly developing fuel cell technology holds the promise to produce abundant energy from hydrogen while producing essentially no greenhouse gases or pollutants (water is the primary reaction product). The importance of hydrogen fuel and fuel cell technology is reflected in U.S. President George W. Bush's 2002 State of the Union Address, in which he stated: “Tonight, I am proposing $1.2 billon in research funding so that America can lead the world in developing clean, hydrogen-power automobiles.” This program has come to be termed “The Hydrogen Fuel Initiative.”

However, if the promise of hydrogen as a fuel, and fuel cell technology, is to be fulfilled, more environmentally friendly sources and methods of producing hydrogen must be developed. Currently, hydrogen is typically produced through the steam reforming of natural gas or other fossil fuels. Not only are these sources non-renewable and in limited supply, but current gasification and reforming technologies for producing hydrogen from such sources produce the greenhouse gas CO₂ as a primary by-product, as well as other pollutant gases, e.g. NOₓ, which are typically released to the atmosphere. Thus, current hydrogen production technologies substantially attenuate and undermine the promise of hydrogen as a clean, abundant source of energy for the future. What is needed are new sources for the production of hydrogen, and methods for producing hydrogen from them, that are clean and/or renewable and that reduce or eliminate net production and additional release to the atmosphere of greenhouse gases such as CO₂.

### SUMMARY OF THE INVENTION

Certain embodiments and aspects of the present invention relate to methods and systems for producing hydrogen using photosynthetic organisms, such as algae, and/or from biomass produced therefrom, especially, in certain embodiments, biomass produced by and harvested from photobioreactors. In certain embodiments, systems and methods are provided whereby hydrogen is produced from biomass produced in photobioreactors that form part of an integrated combustion/gas-treatment/carbon fuel recycling/hydrogen production system.

In a first set of embodiments, a series of gas-treatment and/or hydrogen production systems is disclosed. In one embodiment, a hydrogen production system is disclosed comprising: a photobioreactor system comprising a plurality of enclosed photobioreactor apparatuses (i.e. a plurality of enclosed photobioreactors) and containing a liquid medium therein comprising at least one species of photosynthetic organisms, at least a portion of at least one photobioreactor apparatus being configured to transmit light to the photosynthetic organisms, and the photobioreactor system comprising an inlet configured to be connectable to a source of gas to be treated and an outlet configured to release treated gas from the photobioreactor system; and a hydrogen generating system configured to produce hydrogen gas from biomass comprising photosynthetic organisms harvested from the photobioreactor system.

In another set of embodiments, a system for producing hydrogen is disclosed. The system comprises: a photobioreactor; means for propagating at least one species of photosynthetic organisms within the photobioreactor; means for exposing at least a portion of the photobioreactor and the at least one species of photosynthetic organisms to sunlight as a source of light driving photosynthesis; means for harvesting biomass comprising photosynthetic organisms from the photobioreactor; and means for forming hydrogen gas from harvested biomass.

In another set of embodiments, methods of producing hydrogen using photosynthetic organisms and/or from biomass derived therefrom and/or for treating a gas with a photobioreactor are disclosed. In one embodiment, a method of producing hydrogen comprising acts of: growing at least one species of algae in an enclosed photobioreactor system exposed to sunlight as a source of light driving photosynthesis; and generating hydrogen with the algae is disclosed.

In another embodiment, a method of producing hydrogen comprising acts of growing at least one species of algae in a photobioreactor system comprising a plurality of enclosed photobioreactor apparatuses; and generating hydrogen with the algae is disclosed.

In another embodiment, a method of producing hydrogen is disclosed. The method comprises acts of: providing a liquid medium comprising at least one species of photosynthetic organisms within an enclosed photobioreactor; exposing at least a portion of the photobioreactor and the at least one species of photosynthetic organisms to sunlight as a source of light driving photosynthesis; harvesting at least a portion of the photosynthetic organisms from the bioreactor to form biomass; and generating hydrogen from the biomass.

In another embodiment, a method of producing hydrogen is disclosed. The method comprises acts of: providing a liquid medium comprising at least one species of photosynthetic organisms within a photobioreactor system comprising a plurality of enclosed photobioreactor apparatuses; exposing at least a portion of at least one of the photobioreactor apparatuses and the at least one species of photosynthetic organisms therein to sunlight as a source of light driving photosynthesis; harvesting at least a portion of the photosynthetic organisms from a bioreactor exposed to the sunlight to form biomass; and generating hydrogen from the biomass.

In yet another embodiment, a method for facilitating the production of hydrogen is disclosed. The method comprises an act of: providing biomass produced in an enclosed photobioreactor exposed to sunlight as a source of light driving photosynthesis.

In yet another embodiment, a method for facilitating the production of hydrogen is disclosed. The method comprises an act of: providing biomass produced in a photobioreactor system comprising a plurality of enclosed photobioreactor apparatuses.

In another embodiment, a method of producing hydrogen is disclosed. The method comprises acts of: obtaining biomass produced in an enclosed photobioreactor exposed to sunlight as a source of light driving photosynthesis; and generating hydrogen from the biomass.

In another embodiment, a method of producing hydrogen is disclosed. The method comprises acts of: obtaining biomass produced in a photobioreactor system comprising a plurality of enclosed photobioreactor apparatuses; and generating hydrogen from the biomass.

In another series of embodiments, integrated combustion/gas-treatment/carbon fuel recycling/hydrogen production methods and systems are disclosed. In one such embodiment, an integrated combustion and hydrogen production method is disclosed. The method comprises acts of: burning a fuel with a combustion device to produce a combustion gas stream; passing the combustion gas to an inlet of an enclosed photobioreactor containing a liquid medium therein comprising at least one species of photosynthetic organisms and exposed to sunlight as a source of light driving photosynthesis; at least partially removing at least one substance from the combustion gas with the photosynthetic organisms, the at least one substance being utilized by the organisms for growth and reproduction; removing at least a portion of the at least one species of photosynthetic organisms from the photobioreactor to form a biomass product; and using at least a portion of the biomass product to produce hydrogen gas.

In another embodiment, an integrated combustion and hydrogen production method is disclosed comprising acts of: burning a fuel with a combustion device to produce a combustion gas stream; passing the combustion gas to an inlet of a photobioreactor system comprising a plurality of enclosed photobioreactor apparatuses and containing a liquid medium therein comprising at least one species of photosynthetic organisms; at least partially removing at least one substance from the combustion gas with the photosynthetic organisms, the at least one substance being utilized by the organisms for growth and reproduction; removing at least a portion of the at least one species of photosynthetic organisms from the photobioreactor system to form a biomass product; and using at least a portion of the biomass product to produce hydrogen gas.

### BRIEF DESCRIPTION OF THE DRAWINGS

Other advantages, novel features, and uses of the invention will become more apparent from the following detailed description of non-limiting embodiments of the invention when considered in conjunction with the accompanying drawings, which are schematic and which are not intended to be drawn to scale. In the figures, each identical, or substantially similar component that is illustrated in various figures is typically represented by a single numeral or notation. For purposes of clarity, not every component is labeled in every figure, nor is every component of each embodiment of the invention shown where illustration is not necessary to allow those of ordinary skill in the art to understand the invention. In the drawings:

FIG. 1 is a schematic, cross-sectional view of a tubular, triangular photobioreactor, according to one embodiment of the invention;

FIG. 2 is a schematic front perspective view of a multi-photobioreactor gas treatment array employing ten of the photobioreactors of FIG. 1 arranged in parallel, according to one embodiment of the invention;

FIG. 3 is a schematic right side perspective view of an annular photobioreactor, according to one embodiment of the invention;

FIG. 3a is a cross-sectional view of the annular photobioreactor of FIG. 3, taken along lines 3a-3a;

FIGS. 4a-4g are schematic, cross-sectional views of a variety of photobioreactor configurations;

FIGS. 5a-5g are schematic, cross-sectional views of a variety of annular photobioreactor configurations;

FIG. 6a is a schematic diagram of a photobioreactor system employing the photobioreactor of FIG. 1 and including a computer-implemented control system, according to one embodiment of the invention;

FIG. 6b is a graph illustrating an algae growth curve;

FIG. 7a is a block flow diagram illustrating one embodiment of a method for operating the computer-implemented control system of the photobioreactor system of FIG. 6a;

FIG. 7b is a block flow diagram illustrating another embodiment of a method for operating the computer-implemented control system of the photobioreactor system of FIG. 6a;

FIG. 8a is a block flow diagram illustrating one embodiment of a method for pre-conditioning an algal culture, according to one embodiment of the invention;

FIG. 8b is a block flow diagram illustrating one embodiment of a method for performing step 807 of FIG. 8a;

FIG. 8c is a block flow diagram illustrating one embodiment of a method for performing step 807c of FIG. 8b;

FIG. 8d is a schematic process flow diagram of one embodiment of an automated cell culture adaptation system;

FIG. 8e is a perspective view from the top of one embodiment of a cell culture module of FIG. 8d;

FIG. 8f is a perspective view from the bottom the cell culture module of FIG. 8e;

FIG. 8g, is a schematic plan view of one embodiment of a chopper wheel that forms part of the light source modulator of FIG. 8d; and

FIG. 9 is a schematic process flow diagram of one embodiment of an integrated combustion method and system, according to one embodiment of the invention; and

FIG. 10 is a schematic process flow diagram of certain embodiments of hydrogen production methods and systems that can, in certain embodiments, form part of an integrated combustion method and system, such as that illustrated in FIG. 9.

### DETAILED DESCRIPTION OF THE INVENTION

Certain embodiments and aspects of the present invention relate to photobioreactor apparatus designed to contain a liquid medium comprising at least one species of photosynthetic organism therein, and to methods of using the photobioreactor apparatus as part of a hydrogen production process and/or gas-treatment process and system able to at least partially remove certain undesirable pollutants from a gas stream. In certain embodiments, the disclosed photobioreactor apparatus, methods of using such apparatus, and/or hydrogen production and gas treatment systems and methods provided herein can be utilized as part of an integrated combustion method and system, wherein photosynthetic organisms utilized within the photobioreactor at least partially remove certain pollutant compounds contained within combustion gases, e.g. CO₂ and/or NOₓ, and are, optionally, subsequently harvested from the photobioreactor and processed, and are utilized to produce hydrogen and/or as a fuel source for a combustion device (e.g. an electric power plant generator and/or incinerator). Such uses of certain embodiments of the invention can provide an efficient means for producing hydrogen and/or recycling carbon contained within a combustion fuel (i.e. by converting CO₂ in a combustion gas to biomass in a photobioreactor, and, in certain embodiments, converting this biomass to hydrogen fuel in a hydrogen production system), thereby reducing both CO₂ emissions and fossil fuel requirements for a given quantum of energy produced. In certain embodiments, a photobioreactor apparatus can be combined with a supplemental gas treatment apparatus to effect removal of other typical combustion gas/flue gas contaminants, such as SOₓ, mercury, and/or mercury-containing compounds. In certain embodiments, a photobioreactor apparatus can comprise and/or be combined with a hydrogen generation system configured to generate hydrogen, for example from biomass produced in and harvested from the photobioreactor.

In certain embodiments a control system and methodology is utilized in the operation of a photobioreactor, which is configured to enable automatic, real-time, optimization and/or adjustment of operating parameters to achieve desired or optimal photomodulation and/or growth rates for a particular environmental operating conditions. In yet another aspect, the invention involves methods and systems for pre-selecting, adapting, and conditioning one or more species of photosynthetic organisms to specific environmental and/or operating conditions to which the photosynthetic organisms will subsequently be exposed during utilization in a photobioreactor apparatus of a gas treatment system.

Certain aspects of the invention are directed to photobioreactor designs and to methods and systems utilizing photobioreactors. A “photobioreactor,” or “photobioreactor apparatus,” as used herein, refers to an apparatus containing, or configured to contain, a liquid medium comprising at least one species of photosynthetic organism and having either a source of light capable of driving photosynthesis associated therewith, or having at least one surface at least a portion of which is partially transparent to light of a wavelength capable of driving photosynthesis (i.e. light of a wavelength between about 400-700 nm). Preferred photobioreactors for use herein comprise an enclosed bioreactor system, as contrasted with an open bioreactor, such as a pond or other open body of water, open tanks, open channels, etc.

The term “photosynthetic organism” or “biomass,” as used herein, includes all organisms capable of photosynthetic growth, such as plant cells and micro-organisms (including algae and euglena) in unicellular or multi-cellular form, that are capable of growth in a liquid phase (except that the term “biomass,” when appearing in the titles of documents referred to herein or in such references that are incorporated by reference, may be used to more generically to refer to a wider variety of plant and/or animal-derived organic matter). These terms may also include organisms modified artificially or by gene manipulation. While certain photobioreactors disclosed in the context of the present invention are particularly suited for the cultivation of algae, or photosynthetic bacteria, and while in the discussion below, the features and capabilities of certain embodiments that the inventions are discussed in the context of the utilization of algae (i.e. algal biomass) as the photosynthetic organisms, it should be understood that, in other embodiments, other photosynthetic organisms may be utilized in place of or in addition to algae. For an embodiment utilizing one or more species of algae, algae of various types, (for example Chlorella, Spirolina, Dunaliella, Porphyridum, etc) may be cultivated, alone or in various combinations, in the photobioreactor.

The phrases of “at least partially transparent to light” and “configured to transmit light,” when used in the context of certain surfaces or components of a photobioreactor, refers to such surface or component being able to allow enough light energy to pass through, for at least some levels of incident light energy exposure, to drive photosynthesis within a photosynthetic organism.

The term “hydrogen,” as used herein, unless otherwise noted, refers to molecular hydrogen (e.g. H₂) and does not refer to hydrogen atoms associated with and chemically bound to other, non-hydrogen, atoms in a chemical compound. Thus, for example, the phrase “generating hydrogen,” as used herein, would encompass the production of molecular hydrogen in any phase or form, whether in a pure state or in a mixture, solution, suspension, dispersion, etc. with other materials, but would not encompass the production of hydrogen atom/non hydrogen atom-containing molecules, such as, e.g. H₂O, hydrocarbons, alcohols, ethers, esters, aldehydes, ketones, phenols, amines, carbonyls, nitrites, nitro compounds, organic acids, metal hydrides, etc. which may include hydrogen atom(s) as part of their chemical structure.

The phrase “generating hydrogen from,” or “producing hydrogen from,” or “form(ing) hydrogen from,” as used herein in the context of the production of hydrogen from biomass, refers to the conversion, by chemical reaction, biological digestion, etc. of the biomass, or at least a portion thereof, into a non-biomass product, at least a portion of which comprises hydrogen. This is to be distinguished from production, generation, formation, etc. of hydrogen “with” photosynthetic organisms/biomass, as used herein, which is used to refer to a broader genus of hydrogen production utilizing photosynthetic organisms/biomass, and which describes production and release of hydrogen by the photosynthetic organisms themselves during, and as a product of their, metabolism, as well as conversion, by chemical reaction, biological digestion, etc. of the biomass, or at least a portion thereof, into a non-biomass product, at least a portion of which comprises hydrogen.

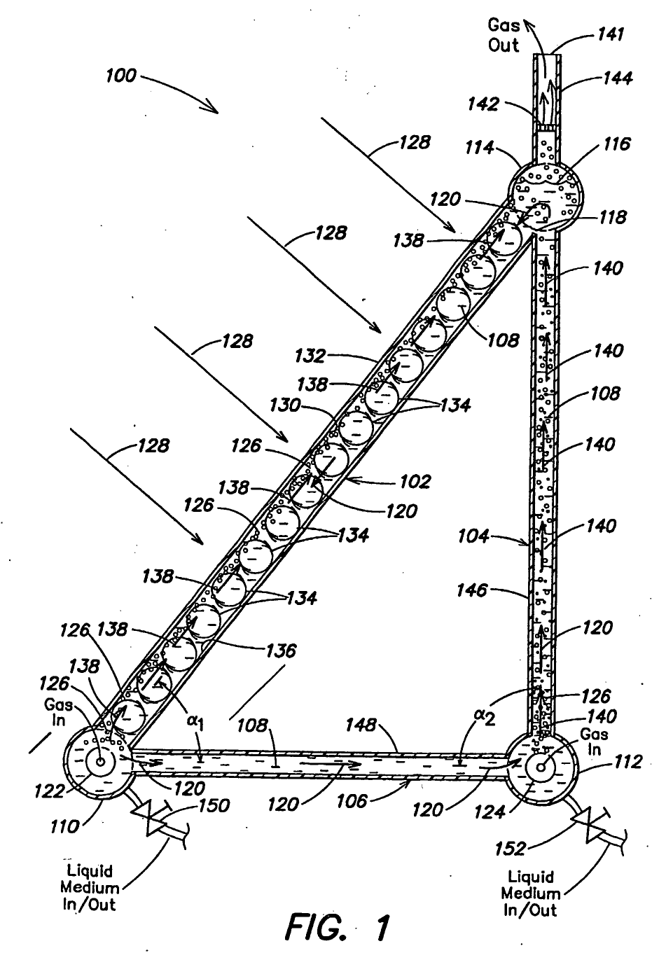

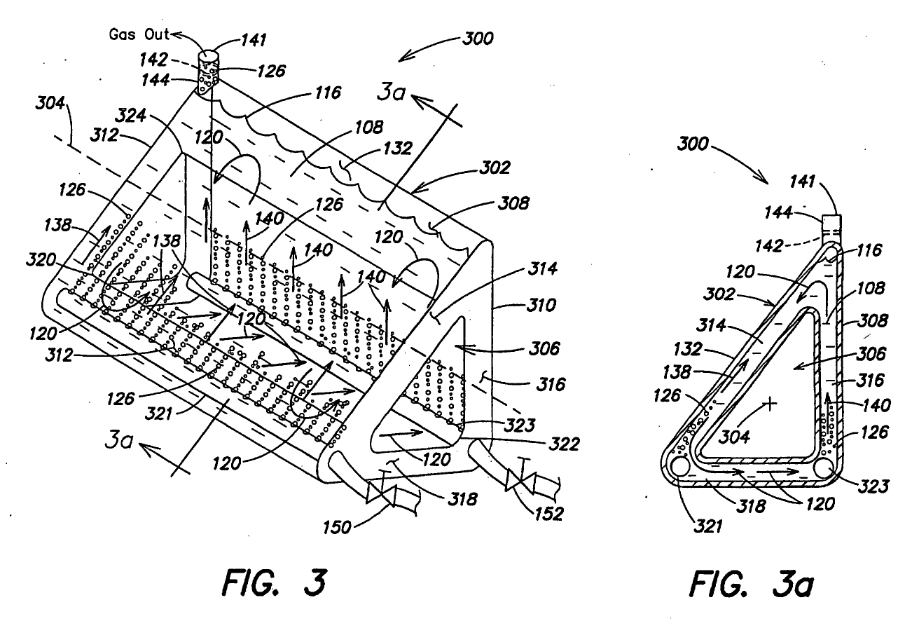

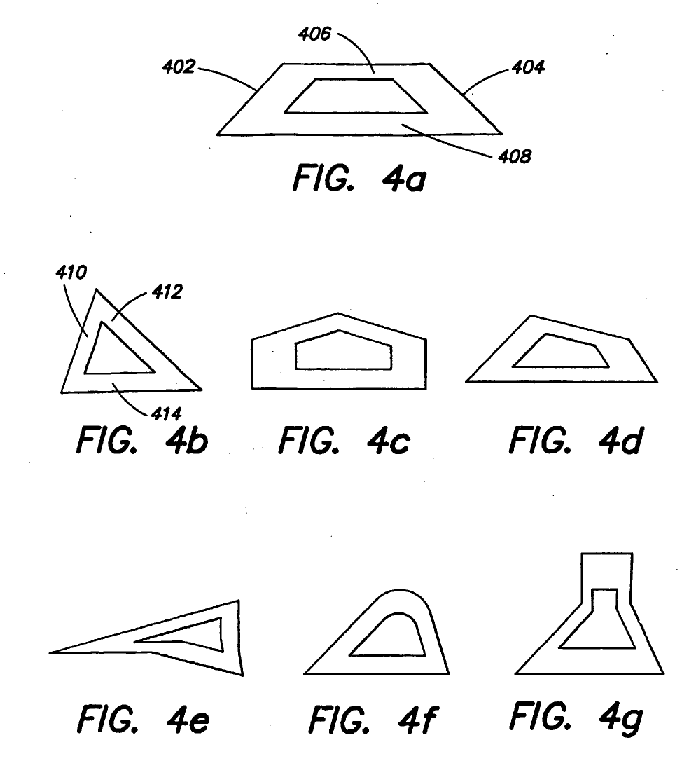

FIG. 1 illustrates one exemplary embodiment of a tubular, loop photobioreactor apparatus 100, according to one aspect of the invention. Photobioreactor 100 comprises three fluidically interconnected conduits 102, 104, and 106, which together provide a flow loop enabling the liquid medium 108 contained within the photobioreactor to flow sequentially from a region of origin (e.g. header or sump 110) within the flow loop, through the three conduits around the loop, and back to the region of origin. While, in the illustrated embodiment, the tubular, loop photobioreactor includes three fluidically interconnected conduits forming the recirculation flow loop, in other embodiments, for example as illustrated in FIGS. 3 and 4 discussed below, the photobioreactor can include four or more fluidically inter-connected conduits forming the flow loop and/or can be arranged having a geometry other than the triangular geometry illustrated in the figure. In yet other embodiments, certain advantages of this aspect of the present invention can be realized utilizing a photobioreactor comprising only two fluidically interconnected conduits or, in yet other embodiments, only a single conduit.

Tubular conduits 102, 104, and 106 are fluidically interconnected via connecting headers 110, 112, and 114, to which the ends of the various conduits are sealingly connected, as illustrated. In other embodiments, as would be apparent to those skilled in the art, other connecting means may be utilized to interconnect the liquid medium-containing conduits, or alternatively, the flow loop could be formed from a single tubular conduit, which is bent or otherwise formed into a triangular, or other shape forming the flow loop.

The term “fluidically interconnected”, when used in the context of conduits, chambers, or other structures provided according to the invention that are able to contain and/or transport gas and/or liquid, refers to such conduits, containers, or other structures being of unitary construction or connected together, either directly or indirectly, so as to provide a continuous flow path from one conduit, etc. to the others to which they are fluidically interconnected in at least a partially fluid-tight fashion. In this context, two conduits, etc. can be “fluidically interconnected” if there is, or can be established, liquid and/or gas flow through and between the conduits (i.e. two conduits are “fluidically interconnected” even if there exists a valve between the two conduits that can be closed, when desired, to impede fluid flow therebetween).

As discussed in greater detail below, the liquid medium contained within the photobioreactor during operation typically comprises water or a saline solution (e.g. sea water or brackish water) containing sufficient nutrients to facilitate viability and growth of algae and/or other photosynthetic organisms contained within the liquid medium. As discussed below, it is often advantageous to utilize a liquid medium comprising brackish water, sea water, or other non-portable water obtained from a locality in which the photobioreactor will be operated and from which the algae contained therein was derived or is adapted to. Particular liquid medium compositions, nutrients, etc. required or suitable for use in maintaining a growing algae or other photosynthetic organism culture are well known in the art. Potentially, a wide variety of liquid media can be utilized in various forms for various embodiments of the present invention, as would be understood by those of ordinary skill in the art. Potentially appropriate liquid medium components and nutrients are, for example, discussed in detail in: Rogers, L. J. and Gallon J. R. “Biochemistry of the Algae and Cyanobacteria,” Clarendon Press Oxford, 1988; Burlew, John S. “Algal Culture: From Laboratory to Pilot Plant.” Carnegie Institution of Washington Publication 600. Washington, D.C., 1961 (hereinafter “Burlew 1961”); and Round, F. E. The Biology of the Algae. St Martin's Press, New York, 1965; each incorporated herein by reference).

Photobioreactor 100, during operation, should be filled with enough liquid medium 108 so that the fill level 116 is above the lower apex 118 of the connecting joint between conduit 102 and conduit 104, so as to permit a recirculating loop flow of liquid medium (e.g. in the direction of arrows 120) during operation. As is explained in more detail below, in certain embodiments, a gas injection and liquid flow inducing means is utilized enabling the liquid flow direction to be either counter-clockwise, as illustrated, or clockwise, or, in yet other embodiments, essentially stagnant. In the illustrated embodiment, as described in more detail below, photobioreactor 100 employs a feed gas introducing mechanism and liquid medium flow-inducing mechanism comprising two gas spargers 122 and 124, which are configured to create a plurality of bubbles 126 rising up and through conduits 102 and 104, thereby inducing liquid flow.

In certain embodiments, photobioreactor apparatus 100, is configured to be utilized in conjunction with a source of natural light, e.g. sunlight 128. In such an embodiment, at least one of conduits 102, 104, and 106 should be at least partially transparent to light of a wavelength capable of driving photosynthesis. In the illustrated embodiment, conduit 102 comprises a “solar panel” tube that is at least partially transparent to sunlight 128, and conduits 104 and 106 have at least a portion of which that is not transparent to the sunlight. In certain embodiments, essentially the entirety of conduits 104 and 106 are not transparent to sunlight 128, thereby providing “dark tubes.”

For embodiments where conduit 102 is at least partially transparent to sunlight 128, conduit 102 may be constructed from a wide variety of transparent or translucent materials that are suitable for use in constructing a bioreactor. Some examples include, but are not limited to, a variety of transparent or translucent polymeric materials, such as polyethylenes, polypropylenes, polyethylene terephthalates, polyacrylates, polyvinylchlorides, polystyrenes, polycarbonates, etc. Alternatively, conduit 102 can be formed from glass or resin-supported fiberglass. Preferably, conduit 102, as well as non-transparent conduits 104 and 106 are sufficiently rigid to be self-supporting and to withstand typical expected forces experienced during operation without collapse or substantial deformation. Non-transparent conduits, e.g. 104 and/or 106, can be made out of similar materials as described above for conduit 102, except that, when they are desired to be non-transparent, such materials should be opaque or coated with a light-blocking material. As will be explained in more detail below, an important consideration in designing certain photobioreactors according to the invention is to provide a desirable level of photomodulation (i.e. temporal pattern of alternating periods of exposure of the photosynthetic organisms to light at an intensity sufficient to drive photosynthesis and to dark or light at an intensity insufficient to drive photosynthesis) within the photobioreactor. By making at least a portion of at least one of the conduits (e.g. conduits 104 and/or 106) non-transparent, dark intervals are built into the flow loop and can help establish a desirable ratio of light/dark exposure of the algae in the photobioreactor leading to improved growth and performance.

While conduits 102, 104, and 106, as illustrated, comprise straight, linear segments, in alternative embodiments, one or more of the conduits may be arcuate, serpentine, or otherwise non-linear, if desired. While, in certain embodiments, tubular conduits 102, 104, and 106 may have a wide variety of cross-sectional shapes, for example, square, rectangular, oval, triangular, etc., in a preferred embodiment, as illustrated, each of the conduits comprises a length of tubing having an essentially circular cross-sectional shape. Additionally, if desired, one or more of conduits 102, 104 and 106 (and especially solar panel conduit 102) can have a variety of flow-disrupting and/or mixing-enhancing features therein to increase turbulence and/or gas-liquid interfacial mixing within the conduit. This can, for example, lead to improved short-duration “flashing light” photomodulation, as explained in more detail below, and/or to improved diffusional uptake of gas within the liquid medium for embodiments wherein the gas to be treated is injected directly into the photobioreactor (e.g., as illustrated in FIG. 1). Such flow enhancements can comprise, but are not limited to, fins, baffles, or other flow directing elements within conduit 102, and/or can comprise providing conduit 102 with a helical twist along its length, etc.

For certain embodiments, (especially for embodiments wherein the gas to be treated, such as combustion gas, flue gas, etc., is injected directly into the photobioreactor at the base of a light-transparent conduit, e.g. conduit 102), performance of the photobioreactor can, in certain situations, be improved by providing certain geometric and structural relationships, as described below.

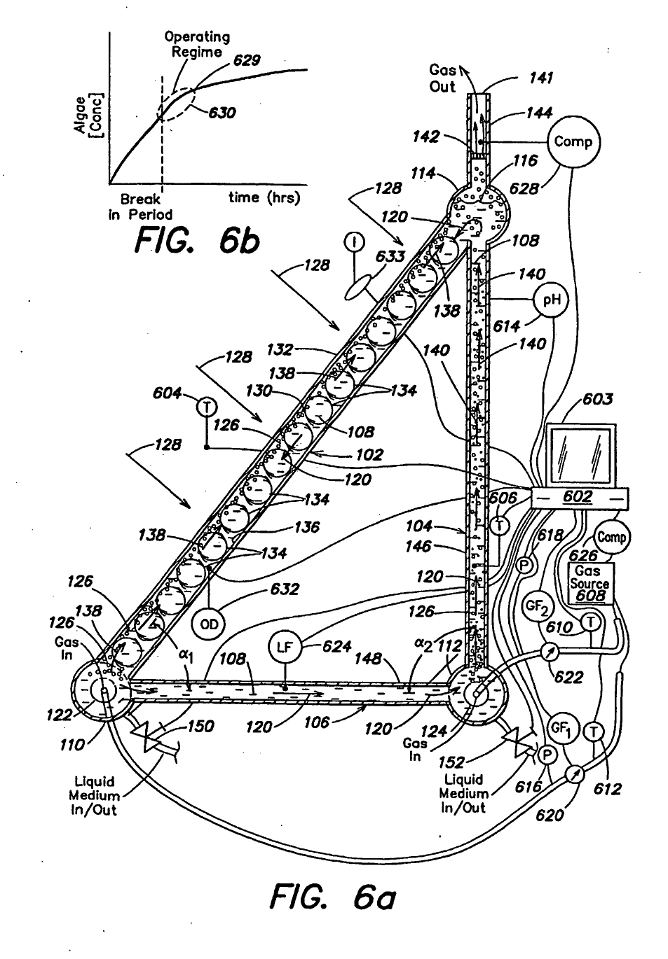

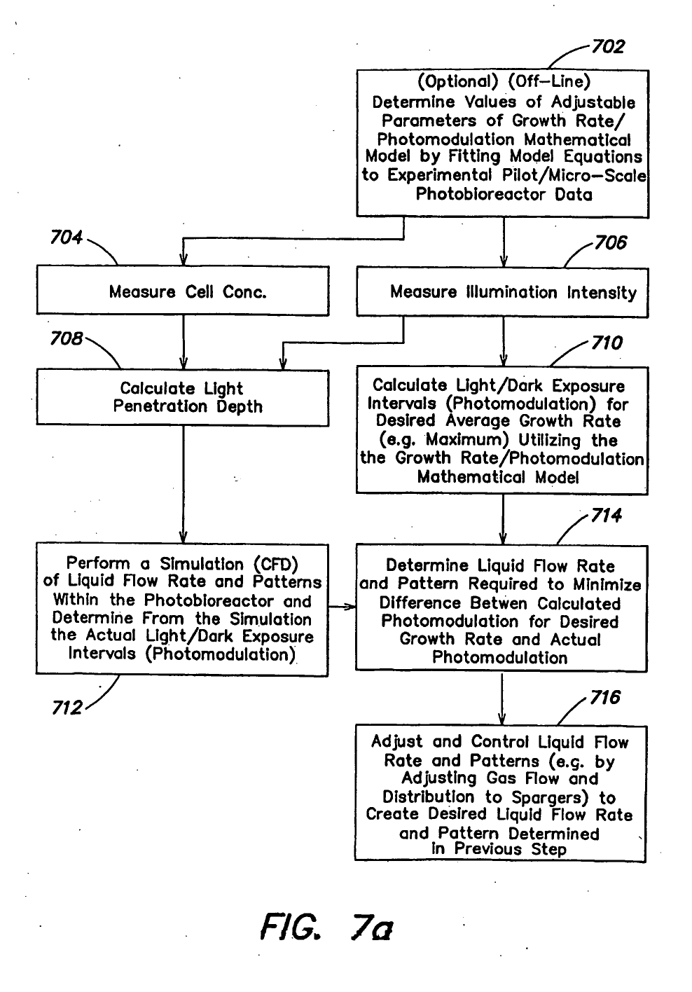

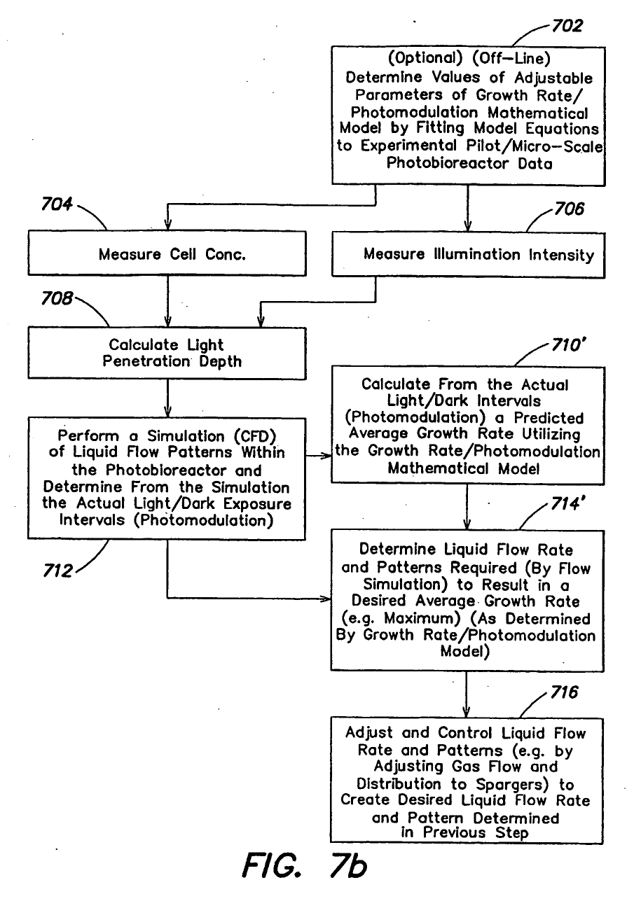

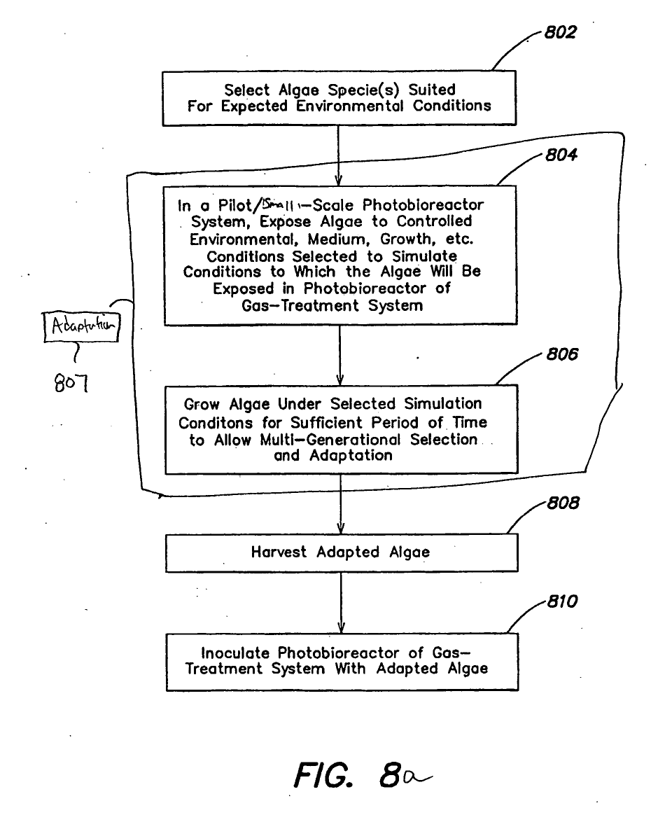

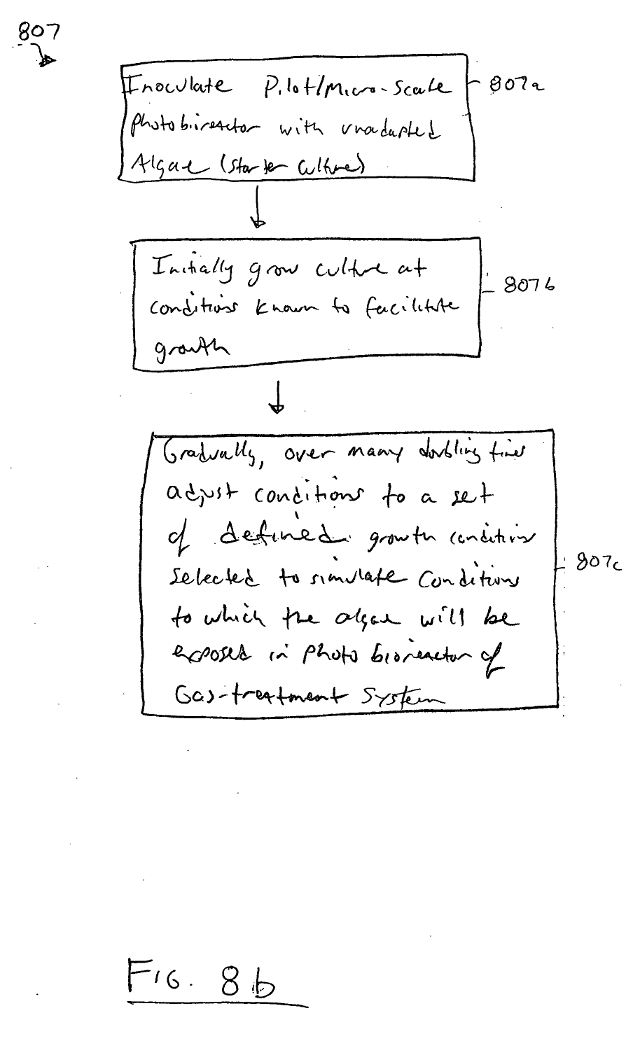

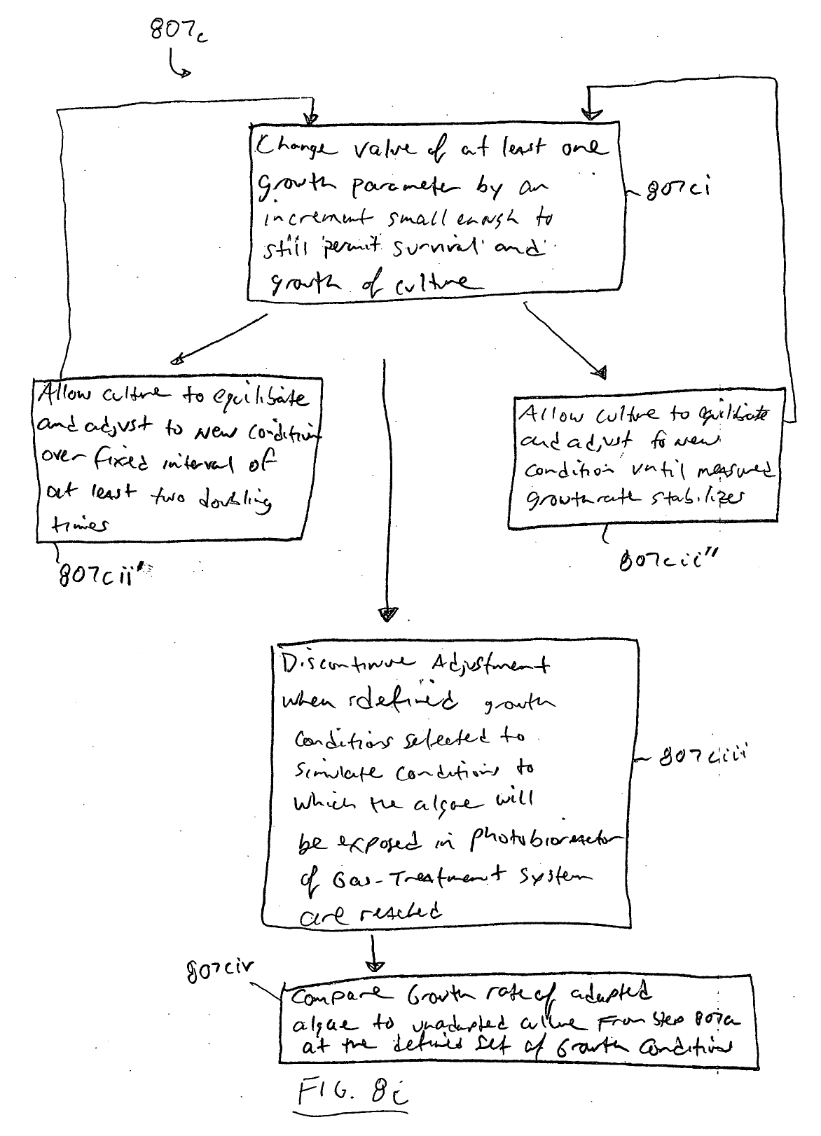

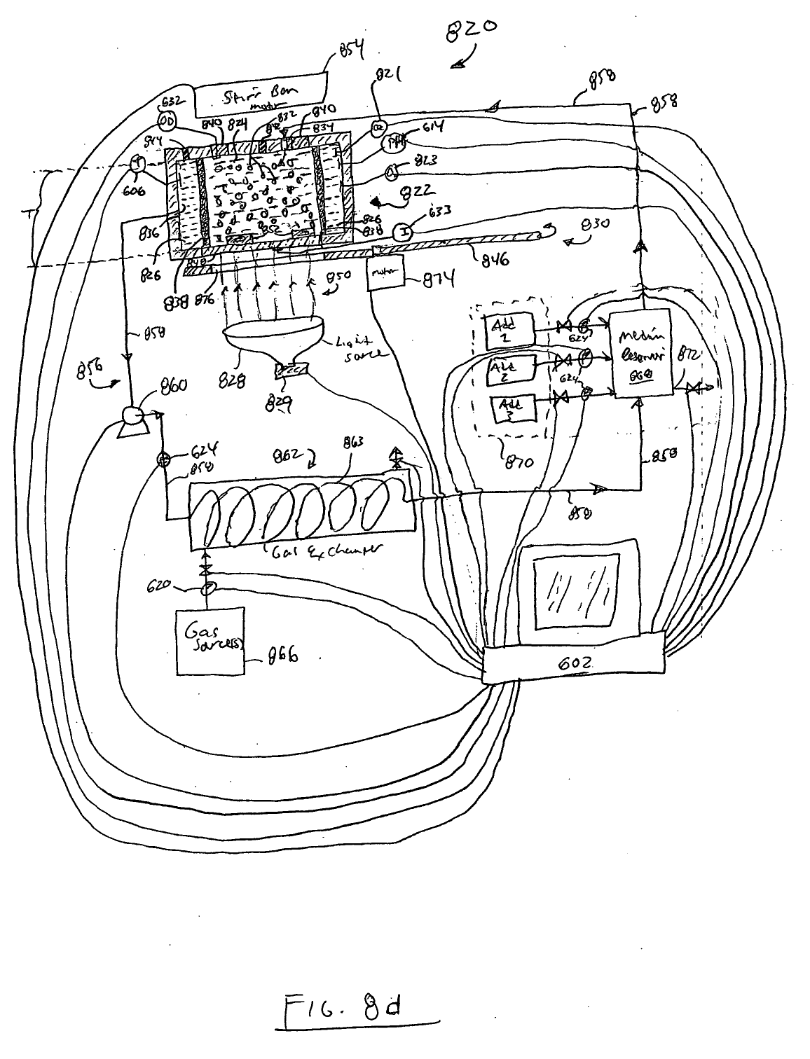

As illustrated, gas sparger 122 is configured and positioned within header 110 to introduce a gas to be treated into the lowermost end of conduit 102, so as to create a plurality of gas bubbles 126 that rise up and through liquid medium 108 contained within conduit 102 along a portion 130 of the inner surface of the conduit that is directly adjacent to that portion 132 of the outer surface of the conduit that most directly faces sunlight 128. This arrangement, in combination with providing certain angles α₁ between conduit 102 and the horizontal plane can enable sparger 122 to introduce the gas stream into the lower end of conduit 102 such that a plurality of bubbles rises up and through the liquid medium inducing a liquid flow within conduit 102 characterized by a plurality of recirculation vortices 134 and/or turbulent eddies positioned along the length of conduit 102. These recirculation vortices and/or eddies both can increase mixing and/or the residence time of contact between the bubbles and the liquid within conduit 102, as well as provide circulation of the algae from light regions near inner surface 130 of conduit 102 to darker regions positioned closer to inner surface 136 of conduit 102, thereby providing a “flashing light” relatively high frequency photomodulation effect that can be very beneficial for the growth and productivity, (i.e. in converting CO₂ to biomass). This effect, and inventive means to control and utilize it, is explained in greater detail below in the context of FIGS. 6a, 7a, and 7b. It is believed that a reason why recirculation vortices 134 and/or turbulent eddies can facilitate enhanced photomodulation is that as the as algae grows within the photobioreactor, the optical density of the liquid medium increases, thereby decreasing the effective light penetration depth within the liquid medium, such that regions within conduit 102 positioned sufficiently far away from inner surface 130 upon which sunlight 128 is incident, will be in regions of the tube where the light intensity is insufficient to drive photosynthesis.

Other advantages of the illustrated arrangement wherein gas sparger 122 and light-transparent conduit 102 are arranged such that gas bubbles 126 rise along the region of the conduit upon which the light is most directly incident include improved cleaning and thermal buffering. For example, as bubbles 126 rise up and along the inner surface 130 of conduit 102, they serve to effectively scour or scrub the inner surface, thereby reducing build up of algae on the surface and/or removing any algae adhered to the surface. In addition, because the bubbles can also be effective at reflecting at least a portion of the light incident upon conduit 102, the bubbles can act to effect a degree of thermal buffering of the liquid medium in the photobioreactor. In some embodiments, to enhance the scrubbing and/or thermal buffering effect of the bubbles, a plurality of neutrally buoyant, optionally transparent or translucent, microspheres (e.g. having a diameter of between 0.5 to about 3 mm) could also be utilized. Such buoyant particles would be carried with the liquid flow within conduit 102, thereby creating an additional scrubbing and/or thermal buffering effect, and/or an additional “flashing light” photomodulation effect.

The term “recirculation vortices” as used herein, refers to relatively stable liquid recirculation patterns (i.e. vortices 134) that are superimposed upon the bulk liquid flow direction (e.g. 120). Such recirculation vortices are distinguishable from typical turbulent eddies characterizing fully developed turbulent flow, in that recirculation vortices potentially can be present even where the flow in the conduit is not fully turbulent. In addition, turbulent eddies are typically relatively randomly positioned and chaotically formed and of, for a particular eddy, short-term duration. As will be explained below, the selection of geometries and liquid and/or gas flow rates within the photobioreactors to create such recirculation vortices and/or turbulent eddies can be determined using routine fluid dynamic calculations and simulations available to those of ordinary skill in the art.

While, in certain embodiments utilizing direct gas injection into the photobioreactor, a single gas sparger or diffuser (e.g., sparger 122) can be utilized, in certain preferred embodiments, as illustrated, the inventive photobioreactor includes two gas spargers 122 and 124, each of which is configured and positioned within the photobioreactor to inject gas bubbles at the base of an upwardly-directed conduit, such as conduit 102 and conduit 104. As will be appreciated by those skilled in the art, the gas bubble stream released from sparger 122 and rising through conduit 102 and the gas bubble stream released from sparger 124 and rising through conduit 104 (in the direction of arrows 138 and 140, respectively), each provide a driving force having a tendency to create a direction of liquid flow around the flow loop that is oppositely directed from that created by the other. Accordingly, by controlling the overall flow rate of a gas to be treated by the photobioreactor and the relative ratio or distribution of the overall flow rate that is directed to sparger 122 and to sparger 124, it is possible to induce a wide variety of pressure differentials within the photobioreactor, which are governed by differences in gas holdups in conduit 102 and conduit 104, so as to drive a bulk flow of the liquid medium either counterclockwise, as illustrated, clockwise, or, with the proper balance between the relative gas injection rates, to induce no bulk liquid flow whatsoever around the flow loop.

In short, the liquid medium fluid dynamics are governed by the ratio of gas flow rates injected into spargers 122 and 124. For example, if all of the gas flow injected into the photobioreactor were injected into one of the spargers, this would create a maximal overall liquid flow rate around the flow loop. On the other hand, there is a certain ratio of distribution that, as mentioned above, would result in a stagnant liquid phase. Thus, the relative bulk liquid flow, the gas-liquid residence time in each of conduits 102 and 104, as well as the establishment of particular liquid flow patterns within the photobioreactor (e.g., recirculation vortices) can be reproducibly controlled via control of the combination of the overall gas flow rate and the relative ratio of the overall gas flow rate injected into each of spargers 122 and 124.

This arrangement can provide a much greater range of flexibility in controlling overall liquid flow rates and liquid flow patterns for a given overall gas flow rate and can enable changes in liquid flow rates and flow patterns within the photobioreactor to be effected without, necessarily, a need to change the overall gas flow rate into the photobioreactor.

Accordingly, as discussed in more detail below in FIG. 6a, control of the gas injection rates into the spargers of such a two-sparger photobioreactor, as illustrated, can facilitate control and management of fluid dynamics within the photobioreactor on two levels, without the need for supplemental liquid recirculation means, such as pumps, etc., thereby enabling control and optimization of photomodulation (i.e., maintaining maximal continuous algae proliferation and growth via controlled light/dark cycling). These two levels of fluid dynamic control enabling photomodulation control comprise: (1) control of the overall liquid flow rate around the flow loop, which controls the relative duration and frequency that the algae is exposed to light in conduit 102 and dark in conduits 104 and 106; and (2) creation and control of rotational vortices and/or turbulent eddies in solar panel conduit 102, in which the algae are subjected to higher frequency variations of light-dark exposure creating, for example, a “flashing light” effect. The liquid flow rate within such a photobioreactor can be adjusted to give a wide range of retention time of the algae within conduit 102 (e.g., in a range of seconds to minutes).

An additional advantage of the two-sparger gas injection embodiment illustrated, is that in one of the conduits in which gas is injected, the relative direction of the gas flow with respect to the direction of bulk liquid flow will be opposite that in the other conduit into which gas is injected. In other words, as illustrated in FIG. 1, gas flow direction 140 in conduit 104 is co-current with the direction of liquid flow 120, while gas flow direction 138 in conduit 102 is counter-current to bulk liquid flow direction 120. Importantly, by providing at least one conduit in which the direction of gas flow is counter-current to the direction of liquid flow, it may be possible to substantially increase the effective rate of mass transfer between the pollutant components of a gas to be injected, (e.g., CO₂, NO_{X}), and the liquid medium.

This can be especially important in the context of NO_{X} removal in the photobioreactor. It has been shown that in bubble column and airlift photobioreactors utilized for NO_{X} removal, a counter-flow-type airlift reactor can have as much as a three times higher NO_{X} removal ability than a reactor in which gas and liquid flow are co-current (Nagase, Hiroyasu, Kaoru Eguchi, Ken-Ichi Yoshihara, Kazumasa Hirata, and Kazuhisa Miyamoto. “Improvement of Microalgal NOₓ Removal in Bubble Column and Airlift Reactors.” Journal of Fermentation and Bioengineering, Vol. 86, No. 4, 421-423. 1998; hereinafter “Hiroyasu et al. 1998”). Because this effect is expected to be more important in the context of NO_{X} removal, where, as mentioned in the background, the rate of uptake and removal is diffusion limited, and since algae can process NO_{X} under both light and dark conditions (i.e., during both photosynthesis and respiration), it may be possible to obtain a similar advantage in NO_{X} removal with the photobioreactor even for a situation wherein the direction of liquid flow 120 is opposite to that illustrated in FIG. 1; i.e. such that the gas and liquid flow in conduit 102 is co-current and the gas and liquid flow in conduit 104 is counter-current. The chemical formula “NO_{X}”, as used herein, refers throughout the present specification to any gaseous compound comprising at least one nitrogen oxide selected from the group consisting of: NO AND NO₂.

The term “gas sparger” or “sparger,” as used herein, refers to any suitable device or mechanism configured to introduce a plurality of small bubbles into a liquid. In certain preferred embodiments, the spargers comprise gas diffusers configured to deliver fine gas bubbles, on the order of about 0.3 mm mean bubble diameter or less, so as to provide maximal gas-to-liquid interfacial area of contact. A variety of suitable gas spargers and diffusers are commercially available and are known to those of ordinary skill in the art.

In the embodiment illustrated in FIG. 1, gas to be treated that is injected into photobioreactor 100 through spargers 122 and 124 makes a single pass through the photobioreactor and is released from the photobioreactor through gas outlet 141. In certain embodiments, a filter 142, such as a hydrophobic filter, having a mean pore diameter less than the average diameter of the algae can be provided to prevent algae from being carried out of the bioreactor through gas outlet 141. In this or alternative embodiments, other well known means for reducing foaming within gas outlet tube 144 and loss of algae through the gas outlet could be employed, as would be apparent to those skilled in the art. As would be apparent to those skilled in the art, and as explained in more detail below, the particular lengths, diameters, orientation, etc. of the various conduits and components of the photobioreactor, as well as the particular gas injection rates, liquid recirculation rates, etc. will depend upon the particular use to which the photobioreactor is employed and the composition and quantity of the gas to be treated. Given the guidance provided herein and the knowledge and information available to those skilled in the arts of chemical engineering, biochemical engineering, and bioreactor design, can readily select dimensions, operating conditions, etc., appropriate for a particular application, utilizing no more than a level of routine engineering and experimentation entailing no undue burden.

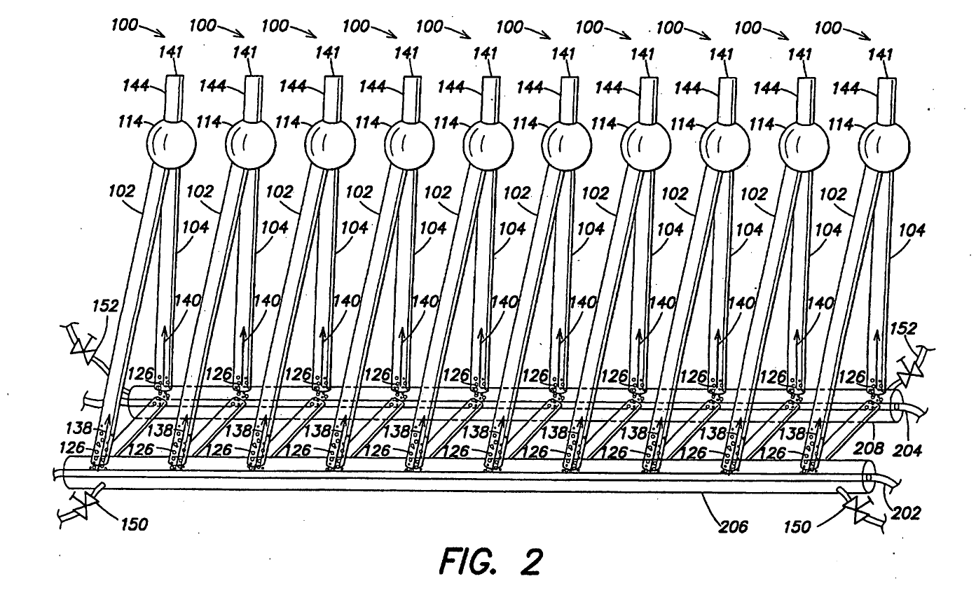

Moreover, as discussed below in the description of FIG. 2, and as would be apparent to those skilled in the art, in certain embodiments, photobioreactor 100 can comprise one of a plurality of identical or similar photobioreactors interconnected in parallel, in series, or in a combination of parallel and series configurations so as to, for example, increase the capacity of the system (e.g., for a parallel configuration of multiple photobioreactors) and/or increase the degree of removal of particular components of the gas stream (e.g., for configurations having gas outlets of a photobioreactor in series with the gas inlet of the same and/or a subsequent photobioreactor). In one such embodiment, a photobioreactor system is designed to separate algae species that are efficient in utilizing NOₓ from species efficient in utilizing CO₂. For example, a nitrogen-efficient algae is placed in a first photobioreactor(s) and carbon-efficient algae is placed in a second photobioreactor(s) in series with the first photobioreactor(s). The flue gas enters the first photobioreactor(s) and is scrubbed of nitrogen (from NOₓ), then flows through the second photobioreactor(s) and is scrubbed of carbon (from CO₂). All such configurations and arrangements of the inventive photobioreactor apparatus provided herein are within the scope of the present invention.

Although photobioreactor 100 was described as being utilized with natural sunlight 128, in alternative embodiments, an artificial light source providing light at a wavelength able to drive photosynthesis may be utilized instead of or in supplement to natural sunlight. For example, a photobioreactor utilizing both sunlight and an artificial light source may be configured to utilize sunlight during the daylight hours and artificial light in the night hours, so as to increase the total amount of time during the day in which the photobioreactor can convert CO₂ to biomass through photosynthesis.

Since different types of algae can require different light exposure conditions for optimal growth and proliferation, in certain embodiments, especially those where sensitive algal species are employed, light modification apparatus or devices may be utilized in the construction of the photobioreactors according to the invention. Some algae species either grow much more slowly or die when exposed to ultraviolet light. If the specific algae species being utilized in the photobioreactor is sensitive to ultraviolet light, then, for example, certain portions of external surface 132 of conduit 102, or alternatively, the entire conduit outer and/or inner surface, could be covered with one or more light filters that can reduce transmission of the undesired radiation. Such a light filter can readily be designed to permit entry into the photobioreactor of wavelengths of the light spectrum that the algae need for growth while barring or reducing entry of the harmful portions of the light spectrum. Such optical filter technology is already commercially available for other purposes (e.g., for coatings on car and home windows). A suitable optical filter for this purpose could comprise a transparent polymer film optical filter such as SOLUS™ (manufactured by Corporate Energy, Conshohocken, Pa.). A wide variety of other optical filters and light blocking/filtering mechanisms suitable for use in the above context will be readily apparent to those of ordinary skill in the art. In certain embodiments, especially for photobioreactors utilized in hot climates, as part of a temperature control mechanism (which temperature control strategies and mechanisms are described in much more detail below in the context of FIG. 6a), a light filter comprising an infrared filter could be utilized to reduce heat input into the photobioreactor system, thereby reducing the temperature rise in the liquid medium.

As discussed above, a particular geometric configuration, size, liquid and gas flow rates, etc. yielding desirable or optimal photobioreactor performance will depend on the particular application for which the photobioreactor is utilized and the particular environmental and operating conditions to which it is subjected. While those of ordinary skill in the art can readily, utilizing the teachings in the present specification, the routine level of knowledge and skill in the art, and readily available information, and utilizing no more than a level of routine experimentation that requires no undue burden, select appropriate configurations, sizes, flow rates, materials, etc. for a particular application, certain exemplary and/or preferred parameters are given below and, more specifically, in the examples at the end of the written description of the application, for illustrative, non-limiting purposes.

In certain embodiments, in order to more readily facilitate the formation of recirculation vortices and/or desirable liquid flow patterns, bubble trajectories, etc., a photobioreactor, such as photobioreactor 100 illustrated in FIG. 1, can be configured so that one or both of angles α₁ and a₂ differ from each other. Preferably, at least one of the conduits forms an angle with respect to the horizontal of greater than 10 degrees and less than 90 degrees, more preferably of greater than 15 degrees and less than 75 degrees, and in certain embodiments of about 45 degrees. Preferably, the angle that falls within the above-mentioned ranges and values comprises the angle between the horizontal and a conduit that is transparent to light and in which photosynthesis takes place, (e.g. angle α₁ between the horizontal and conduit 102). In the illustrated embodiment, conduit 106 has a longitudinal axis that is essentially horizontal. In certain preferred embodiments, α₂ is greater than α₁, and, in the illustrated embodiment, is about 90 degrees with respect to the horizontal.

In certain preferred embodiments, because outer surface 132 of conduit 102 acts as the primary “solar panel” of the photobioreactor, the photobioreactor is positioned, with respect to the position of incident solar radiation 128, such that outer, sun-facing surface 132 of conduit 102 forms an angle with respect to the plane normal to the direction of incident sunlight that is smaller than the angles formed between the sun-facing surfaces 146, 148 of conduits 104 and 106, respectively and the plane normal to the direction of incident sunlight. In this configuration, solar collecting surface 132 is positioned such that sun is most directly incident upon it, thereby increasing solar uptake and efficiency.

The length of gas-sparged conduits 102 and 104 is selected to be sufficient, for a given desired liquid medium circulation rate, to provide sufficient gas-liquid contact time to provide a desired level of mass transfer between the gas and the liquid medium. Optimal contact time depends upon a variety of factors, especially the algal growth rate and carbon and nitrogen uptake rate as well as feed gas composition and flow rate and liquid medium flow rate. The length of conduit 106 should be long enough, when conduit 106 is not transparent, to provide a desired quantity of dark, rest time for the algae but should be short enough so that sedimentation and settling of the algae on the bottom surface of the conduit is avoided for expected liquid flow rates through the conduit during normal operation. In certain preferred embodiments, at least one of conduits 102, 104, and 106 is between about 0.5 meter and about 8 meters in length, and in certain embodiments is between about 1.5 meters and 3 meters in length.

The internal diameter or minimum cross-sectional dimension of conduits 102, 104, and 106, similarly, will depend on a wide variety of desired operating conditions and parameters and should be selected based upon the needs of a particular application. In general, an appropriate inner diameter of conduit 104 can depend upon, for example, gas injection flow rate through sparger 124, bubble size, dimensions of the gas diffuser, etc. If the inner diameter of conduit 104 is too small, bubbles from sparger 124 might coalesce into larger bubbles resulting in a decreased level of mass transfer of CO₂, NOₓ, etc. from the gas into the liquid phase, resulting in decreased efficiency in removing pollutants and/or a decreased level or rate of biomass production.

The inner diameter of conduit 106 can depend upon the liquid medium flow rate and the sedimentation properties of the algae within the photobioreactor, as well as desired light-dark exposure intervals. Typically, this diameter should be chosen so that it is not so large to result in an unduly long residence time of the liquid and algae in conduit 106 such that the algae has time to settle and collect in the bottom of conduit 106 and/or spend too much time during a given flow loop cycle not exposed to light, thereby leading to a reduction in the solar efficiency of the photobioreactor.

The length of conduit 102 is fixed, i.e. by geometry, given a selection of lengths for conduits 104 and 106. However, similar considerations are involved in choosing an appropriate length of conduit 102 as were discussed previously in the context of conduit 104. Regarding the inner diameter of conduit 102, it can be desirable to make this inner diameter somewhat larger than the inner diameters of conduits 104 and 106 (e.g. between about 125% and about 400% of their diameters) to facilitate sufficient light exposure time and to facilitate establishment of recirculation vortices 134. In general, the diameter of conduit 102 can depend upon the intensity of solar radiation 128, algal concentration and optical density of the liquid medium, gas flow rate, and the desired mixing and flow pattern properties of the liquid medium within the conduit during operation. In certain embodiments, the cross-sectional diameter of at least one of conduits 102, 104, and 106 is between about 1 cm and about 50 cm. In certain preferred embodiments, at least one of these diameters is between about 2.5 cm and about 15 cm.

As a specific example, one photobioreactor constructed and utilized by the present inventor comprised an essentially triangular, tubular bioreactor as illustrated in FIG. 1, wherein the fluidically interconnected conduits had an essentially circular cross-sectional shape. The exemplary bioreactor had an angle α₁, of about 45 degrees and an angle α₂ of about 90 degrees, and a conduit 106 that was essentially horizontally oriented. The essentially vertical leg (104) was about 2.2 m in length and about 5 cm in diameter. The essentially horizontal leg (106) was about 1.5 m long and about 5 cm in diameter, and the hypotenuse tube (102) was about 2.6 m long and about 10 cm in diameter. This photobioreactor was used to remove CO₂ and NOₓ from a feed gas mixture comprising 7-15% CO₂, 150-350 ppm NOₓ, 2-10% O₂, with N₂ as the balance fed to the bioreactor at an overall gas flow rate of about 715 ml/min. The total volume of liquid medium in the bioreactor was about 10 liters, and the mean bubble size from the spargers was about 0.3 mm. Concentration of algae (Dunaliella) was maintained at about 1 g (dried weight)/L of liquid medium. Under the above conditions, about 90% CO₂ mitigation, about 98% and about 71% NO_{X} mitigation (in light and dark, respectively), could be achieved with a solar efficiency of about 19.6%.

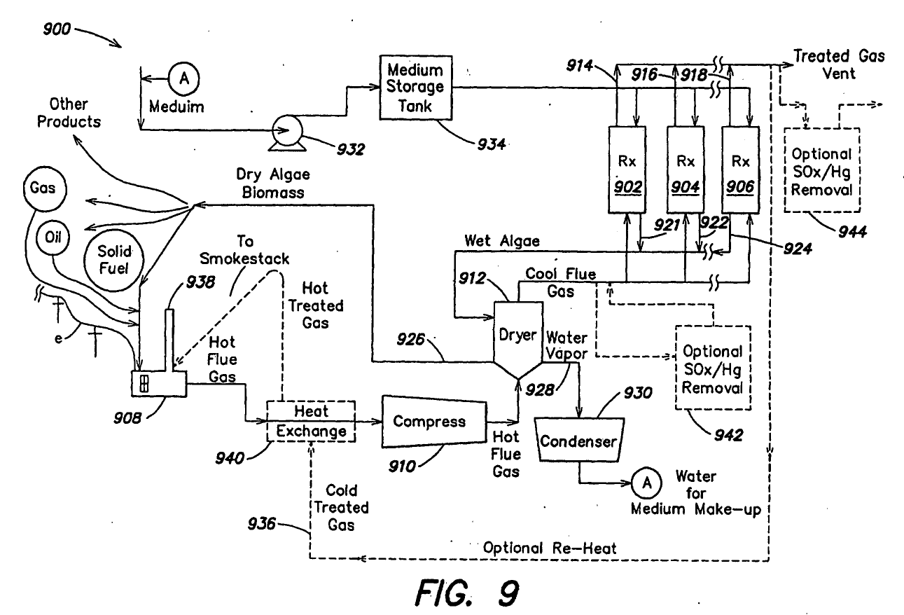

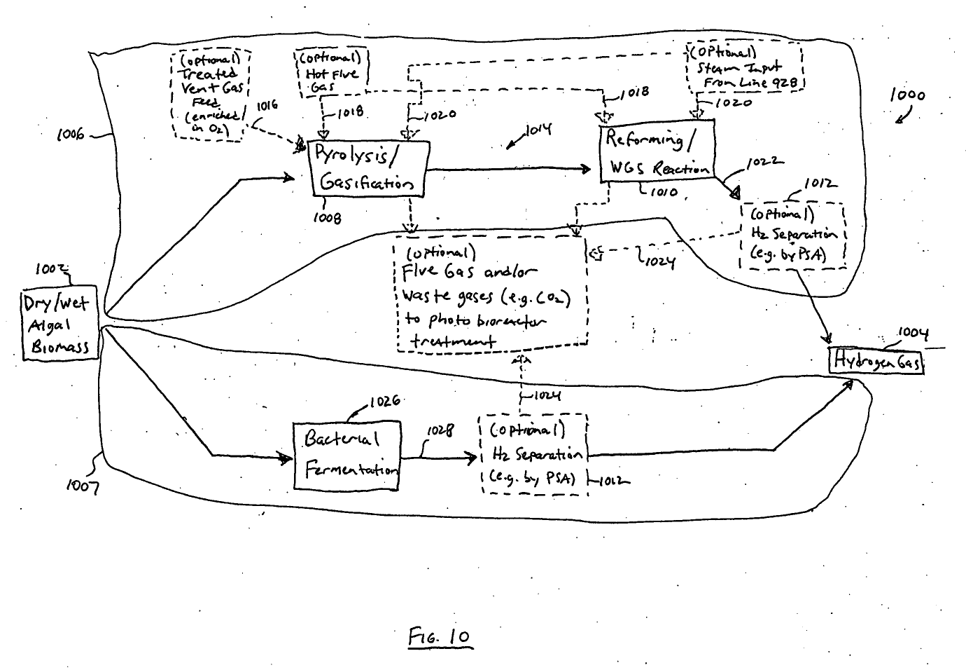

Harvesting algae, adjusting algal concentration, and introducing additional liquid medium can be facilitated via liquid medium inlet/outlet lines 150, 152 as explained in more detail below in the context of the inventive control system for operating the photo bioreactor illustrated in FIG. 6a. Control of the concentration of algae is important both from the standpoint of maintaining a desirable level of algal growth and proliferation as well as providing desirable levels of photomodulation within conduit 102. As explained below, algae is harvested periodically or continuously to maintain the desired concentration range during operation. According to a preferred method, harvesting takes place in a semi-continuous fashion, meaning that only a portion of the algae is removed from the photobioreactor at a given time. To harvest the algae and, sparging is discontinued and the algae are permitted to settle within headers 110 and 112 and conduit 106. Since algae that is denser than the liquid medium will drop to the bottom of the header, gravity can be utilized to harvest the algae; however, flocculants, chemicals that cause algae to clump and settle, may be used, in certain embodiments, to assist in the harvest. Some useful flocculants include clay (e.g. with particle size <2 μm), aluminum sulfate or polyacrylamide. After settling, algae-rich liquid medium can then be withdrawn through one or both of lines 150 and 152. In certain embodiments, fresh, algae-free liquid medium can be injected into one of lines 150 and 152, with the other line open, thereby flushing algae-rich medium out of the photo bioreactor while, simultaneously, replenishing the photobioreactor with fresh medium. In any case, a volume of algae-free fresh liquid medium that is essentially equal to the volume of algae-rich medium withdrawn is added to the photobioreactor before gas sparging is commenced. As explained below in FIG. 9, the water and nutrients contained in the harvested algae can be extracted and recycled to the liquid medium supply of the photobioreactor and/or utilized in the production of hydrogen from the biomass, as illustrated in FIG. 10. This can minimize waste and water use of the photobioreactor and overall system, thereby lowering environmental impact and operational cost.

Certain species of algae are lighter than water and, therefore, tend to float. For embodiments wherein the photo bioreactor is utilized with such species, the algal harvesting process described above could be modified so that after gas sparging is turned off, a sufficient time is permitted to allow algae to float to the top of the photo bioreactor and into header 114. In such an embodiment, a liquid medium outlet/inlet line (not shown) could be provided in header 114 to facilitate removal of the algae-rich liquid medium for harvesting.

In certain embodiments of photobioreactor apparatus provided according to the invention, fouling of the inner surface of the transparent conduit(s) by algal adherence can be reduced or eliminated and cleaning and regeneration of the inner surfaces of the photobioreactor can be facilitated by coating at least the portion of the inner surfaces with a layer of a biocompatible substance that is a solid at temperatures of normal operation (e.g. at temperatures of up to about 45 degrees C.) and that has a melting temperature that is less than the melting temperature of the surface onto which it is coated. Preferably, such substances should also be transparent or translucent such that they do not unduly reduce the transparency of the surface onto which they are coated. Examples of suitable substances can include a variety of waxes and agars. In one variation of such embodiments, a manual or automatic steam sterilization/cleaning procedure can be applied to the photobioreactor after use and prior to a subsequent use. Such a procedure can involve melting and removing the above described coating layer, thereby dislodging any algal residue that adhered thereto. Prior to use, a new coating layer can be applied. This can enable the light transmitting portions of the photo bioreactor to remain clean and translucent over an extended period of use and re-use.

Reference is now made to FIG. 2. FIG. 2 illustrates an embodiment comprising a plurality of photobioreactors 100 (ten as illustrated) arranged in parallel to form a photobioreactor array 200 providing (N) times the gas scrubbing capacity of photo bioreactor 100 (where N=the number of photobioreactors arranged in parallel). Parallel array 200 illustrates a distinct advantage of the tubular photobioreactor apparatus provided according to the invention, namely that the capacity of the photobioreactor system scales linearly with the number of photobioreactor units utilized. Photobioreactor array 200, comprising ten photobioreactor units 100 could share combined gas spargers 202 and 204 and common liquid medium headers/sumps 206 and 208 and can, for example, have a footprint as small as about 1.5 m² or less. As illustrated in the figure, individual photobioreactor units 100 are spaced apart from each other at a greater distance than would typically be the case in a real system for clarity of illustration purposes. Similarly, only a small number of bubbles within the photobioreactors are illustrated, for clarity, and sumps 206 and 208 are illustrated as being transparent, although in a typical system they need not, and typically would not, be. Sumps 206 and 208 should be designed to minimize or eliminate areas of stagnant liquid, which could lead to algal settling and death. In certain preferred systems, individual photobioreactor units 100 will typically be spaced apart from each other on headers 206 and 208 by an essentially minimized distance to reduce to a minimum the open volume within the headers between the photobioreactors. Alternatively, in some embodiments, sumps 206 and 208 may not comprise a simple conduit-like header, as illustrated, but, rather, may comprise a solid structure providing a plurality of cavities located at the points where the various conduits of the photobioreactors connect to the headers, which cavities facilitate fluid communication between the conduits of the individual photobioreactor units, while preventing liquid fluid communication between adjacent photobioreactors.

FIGS. 3 and 3a illustrate an alternative embodiment of a photobioreactor 300, which can have similar geometric and performance characteristics as previously described for tubular photobioreactor 100, while providing the increased gas scrubbing capacity of parallel photobioreactor array 200, while being constructed as a unitary, integral structure. Photobioreactor apparatus 300 comprises an elongated outer enclosure 302, which, when placed on level ground, has an essentially horizontal longitudinal axis 304, and comprises a solar panel surface 132 that is at least partially transparent to light of a wavelength capable of driving photosynthesis. Photobioreactor 300 also includes an elongated inner chamber 306, within elongated outer enclosure 302, having a longitudinal axis that is substantially aligned with longitudinal axis 304 (co-linear as illustrated).

The elongated outer enclosure 302 and the elongated inner chamber 306 together define an annular container 308 that is sealed at its ends by end walls 310 and 312. Annular container 308 provides a flow loop enabling flow of liquid medium 108 contained within the photobioreactor (e.g. in the direction of arrows 120) such that it flows sequentially from a region of origin (e.g. region 312) within the flow loop around the periphery of elongated inner chamber 306 and back to the region of origin. The annular spaces 314, 316, and 318, form three fluidically interconnected conduits akin to conduits 102, 104, and 106 of photobioreactor unit 100 of FIG. 1. Preferably, corners 320, 322, and 324 are somewhat rounded to prevent mechanical damage to algae cells during circulation around the flow loop.

“Substantially aligned with” when used within the above context of the longitudinal axis of the inner chamber being substantially aligned with the longitudinal axis of the outer enclosure, means that the two longitudinal axes are sufficiently parallel and narrowly spaced apart so that the inner chamber and outer enclosure do not come into contact or intersect along any of their faces along the length of the photobioreactor. In certain preferred embodiments, the cross-sectional shape of inner chamber 306 is similar to or essentially the same as that of outer enclosure 308, except proportionally smaller in size. The relative sizes of the inner and outer chamber, the relative spacing and alignment with respect to each other, as well as the shape and orientation of the outer enclosure and inner chamber, all of which factors can dictate the size and spacing of the fluidically interconnected conduits 314, 316, 318 formed by the structure, can be selected and designed considering similar factors as those described previously in the context of the photobioreactor 100. Similarly, materials of construction and the relative transparency or opacity of the various regions and segments of photobioreactor 300 can also be selected considering the above-described disclosure for photobioreactor apparatus 100. For example, even though in FIG. 3 all of the surfaces of photobioreactor 300, except end surfaces 310, are illustrated as being transparent for clarity of illustration, in certain embodiments, the internal and/or external faces defining flow conduits 316 and/or 318 may be rendered non transparent. In certain embodiments, only solar panel 132 is at least partially transparent to the incident light.

Circulation of liquid medium around the flow loop of bioreactor 300 can be facilitated by at least one gas sparger configured to introduce a gas stream into the flow loop of the annular container. In the illustrated embodiment, gas is introduced into both conduits 314 and 316 by elongated tubular gas spargers 321 and 323, which extend along the length of bioreactor 300. Treated gas leaves photobioreactor 300 through gas outlet tube 141.

The length of photobioreactor 300 can be chosen to provide a desired total gas treatment and/or biomass production capacity and is typically limited only by the topography/geometry of the site in which the units 300 are to be located and/or limitations in manufacturing and transportation of the units.

FIGS. 4a-4g illustrate a variety of alternative shapes and configurations for alternative embodiments of photobioreactor 100 and/or photobioreactor 300. FIG. 4a illustrates an essentially trapezoidal configuration, which can have, in an exemplary embodiment, two solar panel conduits 402 and 404 and two dark conduits 406 and 408.

FIG. 4b illustrates an alternative essentially triangular configuration to the essentially right triangle configuration of photobioreactors 100 and 300 illustrated previously. In an exemplary embodiment conduits 410 and 412 could be configured as solar panel conduits with conduit 414 providing a dark leg.

The remaining figures (FIGS. 4c-4g) represent yet additional alternative configurations contemplated by the inventor. The configuration illustrated in FIG. 4e, which has a segmented, non-horizontal non-sparged bottom conduit, could be potentially useful for installations having an irregular or crested terrain. The embodiment in FIG. 4f illustrates a configuration having at least one conduit comprising a curved or arcuate tube and/or surface.

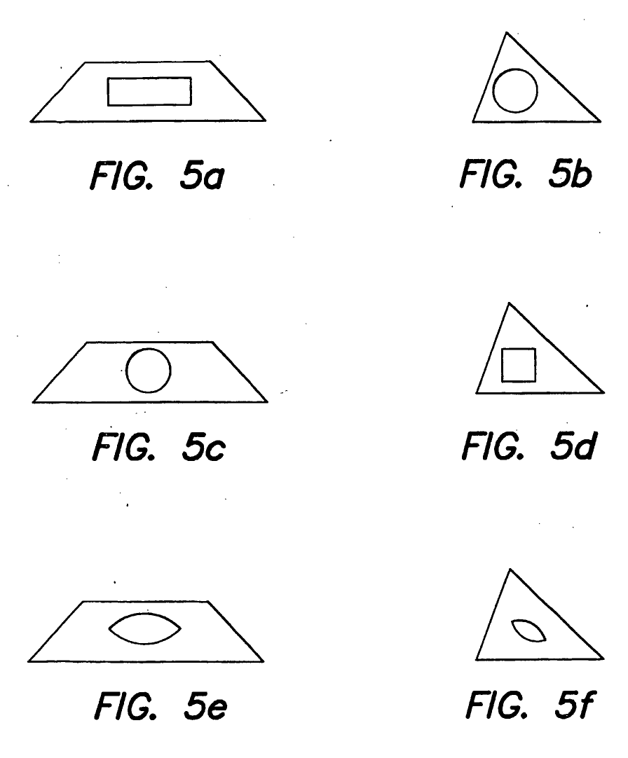

FIGS. 5a-5f illustrate a plurality of alternative configurations, in cross-section, of photobioreactor 300 illustrated previously. In each of the illustrated configurations in FIGS. 5a-5f, the cross-sectional shape of the inner chamber differs from the cross-sectional shape of the outer enclosure, thereby providing flow loops having conduit shapes and dimensions potentially useful for creating desirable recirculation flows and corresponding photomodulation characteristics.

In other aspects, the invention provides systems and methods for treating a gas with a photobioreacto'r including methods for monitoring and controlling liquid flow rates and flow patterns within the photobioreactor to create desired or optimal exposure of the photosynthetic organisms to successive and alternating periods of light and dark exposure to provide a desired or optimal level of photomodulation during operation. It is know that excessive exposure time of algae to light can cause a viability and growth limiting phenomena known as photoinhibition, and that, algal growth and productivity is improved when the algae cells are exposed to both light and dark periods during their growth (i.e. photomodulation). (Burlew 1961; Wu X. and Merchuk J. C. “A model integrating fluid dynamics in photosynthesis and photoinhibition processes,” Chem. Eng. Sci. 56:3527-3538, 2001 (hereinafter “Wu and Merchuk, 2001,” incorporated herein by reference); Merchuk J. C., et al. “Light-dark cycles in the growth of the red microalga Porphyridium sp.,” Biotechnology and Bioengineering, 59:705-713, 1998; Marra, J. “Phytoplankton Photosynthetic Response to Vertical Movement in A Mixed Layer.” Mar. Biol. 46:203, 1978). As illustrated in FIG. 6a, certain aspects of the present invention provide gas treatment systems comprising one or more photobioreactors and further comprising a control system for controlling and/or monitoring various environmental and performance conditions and/or operating parameters of the photobioreactor, as well as implementing the methods for inducing and controlling photomodulation.

Referring to FIG. 6a, a gas treatment system 600 is shown that includes a photobioreactor 100, a plurality of monitoring and control devices, described in more detail below, and a control system comprising a computer implemented system 602 that is configured to control various operating parameters as well as to control flow within the photobioreactor to provide desired or optimal levels of light/dark exposure intervals and frequency to yield desired or optimal levels of photomodulation.

In certain embodiments, as discussed in more detail below in the context of the FIGS. 7a and 7b, the computer implemented system 602 is configured to control photomodulation by: performing a simulation of liquid flow patterns within the photobioreactor; and, from the simulation, to calculate exposure intervals of the photosynthetic organisms to light at an intensity sufficient to drive photosynthesis and to dark or light at an intensity insufficient to drive photosynthesis; and to control the flow of the liquid medium within the photobioreactor so as to yield desired or optimal exposure intervals providing a desired or optimal level of photomodulation. Also, as explained in more detail below, desirable or optimal light/dark exposure intervals are, in certain embodiments, also determined by the computer implemented system utilizing a mathematical model, described in more detail below, of algal growth rate as a function of light/dark exposure intervals.

As used in the above context, an “exposure interval” of a photosynthetic organism to light or dark refers to both length and frequency of exposure to such conditions over a given time period of interest (e.g. a time period required for liquid medium in a tubular flow loop photobioreactor to flow around the entire flow loop). Specifically, as discussed in more detail below, computer implemented system 602, in certain preferred embodiments in calculating “exposure intervals” determines the duration of exposure of the algae, on average, to light intensities both above and below the threshold required to drive photosynthesis as well as the frequency of exposure of the algae to light and dark periods as the algae in the liquid medium is carried around the flow loop of the photobioreactor.

It should be understood that even though the current aspect of the present invention is illustrated utilizing photobioreactor 100 for illustrative purposes, in other embodiments, the photomodulation control methodology and control systems described herein could be utilized with other photobioreactors described herein or other conventional photobioreactors. In certain embodiments, photobioreactors of a design similar to photobioreactor 100 are preferred because of the above-described ability of the photobioreactor to create liquid flow in a solar panel tube, such as tube 102, characterized by recirculating vortices 134 and/or turbulent eddies, which can be effective in subjecting the algae within the tube 102 relatively high frequency cycling between areas of the tube in which light intensity will be sufficient to drive photosynthesis (e.g. near surface 132) and other areas of the tube further away from the surface where light intensity is insufficient to drive photosynthesis.

For example, depending on the relative velocities of the liquid medium flow and gas bubble flow within tube 102, photomodulation frequency (i.e. light to dark interval transition) of greater than 100 cycles per second to less than one cycle per second may be provided. Such a high frequency “flashing light” effect during photosynthetic activity has been found to be very beneficial for growth and productivity of many species of algae (see, Burlew 1961). Moreover, tubes 104 and 106, in certain embodiments, can be made either entirely or partially non-transparent to provide additional, more extended exposure of the algae to dark, rest periods, which can be beneficial for productivity as well.

Before describing the inventive photomodulation control methodology and control system of the photobioreactor system 600, various sensors and controls that can be provided by the photobioreactor system will be explained. Control of certain of the physico-chemical conditions within the photobioreactor can be achieved using conventional hardware or software-implemented computer and/or electronic control systems together with a variety of electronic sensors.

For example, it can be important to control liquid medium temperature within photobioreactor 100 during operation to maintain liquid medium temperature within a range suitable or optimal for productivity. These specific, desirable temperature ranges for operation will, of course, depend upon the characteristics of the algae species used within the photobioreactor systems. Typically, it is desirable to maintain the temperature of the liquid medium between about 5 degrees C. and about 45 degrees C., more typically between about 15 degrees C. and about 37 degrees C., and most typically between about 15 degrees C. and about 25 degrees C. For example, a desirable temperature operating condition for a photobioreactor utilizing Chlorella algae could have a liquid medium temperature controlled at about 30 degrees C. during the daytime and about 20 degrees C. during nighttime.

Gas treatment system 600 can control the liquid medium temperature, in certain embodiments, in one or more ways. For example, the temperature of the liquid medium can be controlled via control of the inlet temperature of the gas to be treated fed to spargers 122 and 124 and/or via supplemental cooling systems for directly cooling photobioreactor 100. Liquid medium temperature can be monitored in one or more places throughout photobioreactor 100 for example by temperature sensors 604 and 606. Feed gas from gas source 608 fed to sparger 122 and sparger 124 can be temperature monitored via temperature sensors 610 and 612, respectively. In certain embodiments, feed gas from gas source 608 is passed through a heat exchanger, for example algal drier 912 illustrated in FIG. 9, prior to injection into photobioreactor 100. Depending on the temperature of the liquid medium detected by temperature sensor 604 and 606, the computer implemented control system 602 can, in certain embodiments, control such a heat exchanger system so as to increase or decrease the temperature of the gas fed to spargers 122 and 124 to raise or lower the temperature of the liquid medium.

As mentioned above, and as explained in more detail below, the demand for cooling and/or heating of the photobioreactor system can be lessened by using an algal strain which has an optimal productivity at temperatures close to actual temperatures to which the algae will be exposed at the operating site. In addition to controlling the liquid medium temperature via modifying the temperature of the feed gas with a heat exchange device, as described above, in other embodiments, especially for embodiments wherein the photobioreactor apparatus is operated in a hot climate, infrared optical filters, as described above, can be utilized to keep heat energy out of the photobioreactor and/or a supplemental cooling system, such as a set of external water sprinklers spraying water on the outside of the photobioreactor, could be utilized to lower temperature.

Liquid medium pH can be monitored via pH probe 614. pH can be controlled at desirable levels for a particular species of algae by, for example, providing one or more injection ports, for example in fluid communication with liquid medium inlet/outlets 150 and/or 152, into which pH adjusting chemicals, such as hydrochloric acid and sodium hydroxide, could be controllably injected.

System 600 can also provide various probes and monitors for measuring the pressure of the feed gas fed to the spargers (e.g. pressure monitors 616 and 618) as well as flow meters for measuring gas flow rates (620, 622), and bulk liquid flow rate within the photobioreactor flow loop (flow meter 624). Gas and liquid flow rates can be controlled, as explained in more detail below, at least in part, to facilitate desired or optimal levels of photomodulation by inducing desirable liquid flow patterns within the photobioreactor. A second control factor dictating the overall flow of gas fed to photobioreactor 100 can be the desired level of removal of pollutants such as CO₂ and/or NOₓ by the photobioreactor. For example, as illustrated, system 600 includes appropriate gas composition monitoring devices 626 and 628 for monitoring the concentration of various gases, such as CO₂, NOₓ, O₂, etc. in the feed gas and treated gas, respectively. Gas inlet flow rate and/or distribution to the spargers can be adjusted and controlled to yield a desirable level of pollutant removal by the photobioreactor system.

As mentioned above, periodically, in order to keep the concentration of algae within the photobioreactor within a range suitable for long term operation and productivity, it can be necessary to harvest at least a portion of the algae and supplement the photobioreactor with fresh, algae-free medium to adjust concentration of algae within the photobioreactor. As illustrated in FIG. 6b, under growth conditions, algae concentration (y axis) will increase exponentially with time (the log growth phase) up to a certain point 629, after which the concentration will tend to level off and proliferation and growth will decrease. In certain preferred embodiments, the concentration of algae within the photobioreactor is maintained within an operating range 630 that is near the upper end of the concentration in which the algae is still in the log growth regime. As would be understood by those by those skilled in the art, the particular growth curve characterizing a given species of algae will be different from species to species and, even within a given a species of algae, may be different depending on differences in operating and environmental factors, (e.g., liquid medium composition, growth temperature, gas feed composition, etc.). As explained in more detail below, in certain embodiments the invention teaches the use of photobioreactor systems using pre-conditioned or pre-adapted algae optimized for growth at the particular operating conditions expected within the photobioreactor gas treatment systems provided according to the invention. In any case, the appropriate algae concentration range which photobioreactor control system 602 should be configured to maintain the photobioreactor should be determined for a particular application by routine testing and optimization. Such routine testing and optimization may take place in a pilot-scale photobioreactor system or in an automated cell culture management system, as are described in more detail below.

Once the desired algae concentration range has been determined, as described above, control system 602 can be configured to control the algal concentration within this range by detecting the algae concentration within the liquid medium, harvesting the algae, and supplementing the system with fresh liquid medium, which harvesting procedure was described in detail previously. In order to determine the concentration of algae within the photobioreactor, a turbidity meter and/or spectrophotometer 632 (or other appropriate optical density or light absorbance measuring device) can be provided. For example, a spectrophotometer could be used to continuously measure the optical density of the liquid medium and evaluate the algal concentration from the optical density according to standard methods, such as described in Hiroyasu et al. 1998.

In general, chemicals for nutrient level maintenance and pH control and other factors could be added automatically directly into the liquid phase within the photobioreactor, if desired. Computer control system 602 can also be configured to control the liquid phase temperature in the photobioreactor by either or both of controlling a heat exchanger system or heat control system within or connected with the photobioreactor, or, in alternative embodiments removing liquid medium from the photobioreactor and passing through a heat exchanger in, for example, a temperature controlled water bath (not shown).

As mentioned above, certain preferred embodiments of photobioreactor gas treatment system 600 include a computer-implemented control system 602 configured for controlling liquid flow patterns within photobioreactor 100 so as to provide desired photo modulation characteristics to provide a desired average algae growth rate, for example a maximum average growth rate achievable. In certain embodiments, the photomodulation control system and methodology utilizes two mathematical models to determine optimal or desired liquid flow patterns for optimizing photomodulation. The first mathematical model involves simulating the growth rate of the algae as a function of sequential and alternating exposure to intervals of light and dark, and the second mathematical model involves a simulation of liquid flow patterns within the photobioreactor as a function of system configuration and geometry and flow rates of liquid medium, (and for systems involving gas injection-driven liquid flow, gas injection rates into the photobioreactor). FIGS. 7a and 7b outline two of the many possible strategies for implementing the above-described photomodulation control scheme with computer-implemented control system 602.

Regarding the above-described mathematical models that can be utilized by control system 602 in optimizing photomodulation, the first mathematical model for correlating light/dark exposure intervals (photomodulation) to average growth rate can, in certain embodiments, be based upon a mathematical model proposed in the literature (see Wu and Merchuk, 2001). The model is based upon the hypothesis that the photosynthetic process in algal cells has three basic modes: (1) activated, (2) resting, and (3) photoinhibited. The fraction of an algal population in each of the three above modes can be represented by x₁, x₂, and x₃ respectively (where x₁+x₂+x₃₌₁).

The model proposes that under normal conditions, an active algal culture reaches photosaturation, becomes photoinhibited and must rest at regular intervals for optimal productivity. In the photoinhibition and resting modes, the culture is unable to use light for carbon fixation. Thus, light exposure during periods of photoinhibition or rest is essentially wasted because it is not available for photosynthesis and carbon fixation and can actually be detrimental to the viability of the culture. The proposed model provides a series of differential, time-dependent equations describing the dynamic process by which the algal culture shifts between the activated, resting, and photoinhibited modes:

$$\begin{matrix}\frac{ⅆ {x}_{1}}{ⅆ t} = - α I {x}_{1} + γ {x}_{2} + δ {x}_{3} & Eq . 1 \\ \frac{ⅆ {x}_{2}}{ⅆ t} = α I {x}_{1} - γ {x}_{2} - β I {x}_{2} & Eq . 2 \\ \frac{ⅆ {x}_{3}}{ⅆ t} = β I {x}_{2} - δ {x}_{3} & Eq . 3\end{matrix}$$

while,
x₁+x₂+x₃=1 Eq. 4
and,
μ=kγ x₂−Me Eq. 5

In these equations, a is a rate constant of photon utilization to transfer the algal culture from x₁ to x₂, β is a rate constant describing transfer from x₂ to x₃, γ is a rate constant describing transfer from mode x₂ to x₁, β is a rate constant describing transfer from x₃ to x₁, μ is the specific growth rate, Me is the maintenance coefficient, and k is the dimensionless yield of photosynthesis production to the transition x₂ to x₁.

In a photobioreactor apparatus such as photobioreactor 100, illumination intensity I will be a complex function of time, depending on the fluid dynamics, light intensity of exposure, and algal concentration within photobioreactor 100.

Illumination I as a function of time (i.e. the time history of illumination intensity of the algae as it flows through the photobioreactor) can be determined, as described in more detail below, utilizing a simulation of the fluid dynamics within the photobioreactor. Once this parameter is determined, and once the constants α, γ, β, k, and Me are determined, specific growth rate μ can be determined for a given illumination history around a flow loop cycle. Solution of these equations can be effected utilizing a wide variety of known numerical techniques for solving differential equations. Such numerical techniques can be facilitated by equation-solving software that is commonly commercially available or can be readily prepared by one of ordinary skill in the art of applied mathematics.

While it can be possible to utilize controlled experiments within a production-scale photobioreactor, such as photo bioreactor 100, to determine the appropriate values of the various constants in the above mathematical model via fitting the model to experimental data, in certain embodiments, for simplicity and accuracy, it may be desirable to utilize a pilot photobioreactor system being able to permit precise and direct manipulate of parameters such as the duration, frequency, and intensity of light exposure of the culture. For example, for a photobioreactor system wherein the algal culture is exposed to an essentially uniform light intensity throughout the entire culture and to a series of essentially identical light/dark exposure cycles (i.e. in which successive light/dark exposure cycles are essentially identical), a quasi-steady state analytical solution of the above-equations is possible. (see, Wu and Merchuk, 2001)

Such an experimental photobioreactor system could comprise, for example, a micro-scale photobioreactor in an automated cell culture system in which the algal cells are subjected to precisely controlled intervals of light and dark exposure at a regular, constant frequency. Alternatively, a pilot-scale, thin-film, tubular loop reactor having fluid flow behavior providing an exact, repetitive light/dark exposure ratio, such as that disclosed in Wu and Merchuk, 2001, could be utilized. Under such quasi-steady state conditions, the mean specific growth rate for one cycle is given by (Wu and Merchuk, 2001):

$$\begin{matrix}\overline{μ} = \frac{k γ}{{t}_{c}} {\int }_{0}^{{t}_{c}} {x}_{2} ( t ) ⅆ t - Me \\ = \frac{k γ}{{t}_{c}} [ {\int }_{0}^{{t}_{1}} {x}_{2 , l} ( t ) ⅆ t + {\int }_{{t}_{1}}^{{t}_{c}} {x}_{2 , d} ( t ) ⅆ t ] - Me \\ = \frac{k γ}{{t}_{c}} [ \frac{c}{b} {t}_{l} + \frac{{C}_{1}}{A} ( s - 1 ) + \frac{{C}_{2}}{B} ( n - 1 ) + ( \frac{c}{b} + {C}_{1} s + {C}_{2} n ) \frac{u - 1}{u γ} - Me\end{matrix} \qquad Eq . 6$$

where,
a=αI+βI+γ+δ,
b=αβI²+δγ+αIδ+βIδ,
c=αIβ;
and

$$\begin{matrix}A = - \frac{a + \sqrt{{a}^{2} - 4 b}}{2} , \\ B = - \frac{a - \sqrt{{a}^{2} - 4 b}}{2}\end{matrix}$$

$$and$$

$${C}_{1} = - \frac{\begin{matrix}Bc ( u - 1 ) ( n - v ) + α Ib ( n - u ) ( v - 1 ) + \\ c ( α I + β I + γ ) ( n - 1 ) ( u - v )\end{matrix}}{\begin{matrix}b [ B ( s - u ) ( n - v ) - A ( n - u ) ( s - v ) + \\ ( α I + β I + γ ) ( s - n ) ( u - v ) ]\end{matrix}}$$

$${C}_{2} = - \frac{\begin{matrix}Ac ( u - 1 ) ( s - v ) + α Ib ( s - u ) ( v - 1 ) + \\ c ( α I + β I + γ ) ( s - 1 ) ( u - v )\end{matrix}}{\begin{matrix}b [ B ( s - u ) ( n - v ) - A ( n - u ) ( s - v ) + \\ ( α I + β I + γ ) ( s - n ) ( u - v ) ]\end{matrix}}$$

$$where$$

$$s = {ⅇ}^{{At}_{1}} , n = {ⅇ}^{{Bt}_{1}} , u = {ⅇ}^{γ {t}_{d}} , v = {ⅇ}^{δ {t}_{d}}$$

In these equations, t is time, t₁ is the time during the cycle in which the algal culture is exposed to light at an intensity capable of driving photosynthesis, td is the time during the cycle during which the algal culture is exposed to dark or light at an intensity incapable of driving photosynthesis and t_{c} is the total cycle time (i.e. t₁+t_{d}).

The above equations describing the analytical may be curve fit to experimental data of algal growth rate as a function of time to determine the values of the various constants (e.g., as described in Wu and Merchuk, 2001). For example, using the above approach, Wu and Merchuk, 2001 determined the following values for the constants in Eqs. 1-5 for a culture of red marine algae, Porphyridium SP (UTEX 637) to be:

TABLE 1
Adjustable Parameter Values and 95% confidence intervals

| Parameter | Value | 95% confidence interval |
|---|---|---|
| α | 0.001935 μE m⁻² | −0.00189-0.00576 |
| β | 5.7848 × 10⁻⁷ μE m⁻² | −0.000343-0.000344 |
| γ | 0.1460 s⁻¹ | −0.133-0.425 |
| δ | 0.0004796 s⁻¹ | −0.284-0.285 |
| k | 0.0003647 | −0.000531-0.00126 |
| | (dimensionless) | |
| Me | 0.05908 h⁻¹ | −0.0126-0.131 |

The mathematical model utilized by computer-implemented control system 602 to determine liquid flow patterns within the photobioreactor as a function of liquid flow rate and/or overall gas injection rate and gas-injection distribution to spargers 122 and 124 can comprise a commercially available Computational Fluid Dynamics (CFD) software package, such as FLUENT™ or FIDAP (Fluent Incorporated, Lebanon, N.H.), or another known software package, or custom-designed CFD software program providing a three-dimensional solution to the Navier-Stokes Equations of Motion (e.g. see, Doering, Charles R. and J. D. Gibbon, Applied Analysis of the Navier-Stokes Equations, Cambridge University Press 2001, incorporated herein by reference). Those of ordinary skill in the art of fluid mechanics and computational fluid dynamics can readily devise such fluid flow simulations and, alone or in combination with one of ordinary skill in the art of computer programming, prepare software to implement such simulations. In such simulations, finite element mathematical techniques may be utilized and such computations may be performed or assisted using a wide variety of readily available general purpose or fluid-flow specific finite element software packages (for example one or more of those available from ALGOR, Inc., Pittsburgh, Pa. (e.g. ALGOR's “Professional Fluid Flow” software package)).

In the photobioreactor system 600 illustrated in FIG. 6a utilizing photobioreactor 100, the CFD simulation performed by computer implemented control system 602 in certain embodiments can determine, for each passage of algae around the flow loop (i.e., each cycle of the algae as it moves around the flow path provided by conduits 106, 104, and 102 of photobioreactor 100), the duration and frequency of the light and dark intervals to which the algae is exposed (i.e. the photomodulation pattern). In certain embodiments, the CFD model can account for the physical geometry of the photobioreactor and the various flow sources and sinks of the photobioreactor to determine the bulk flow and liquid flow patterns of the liquid medium in each of the three legs of photobioreactor 100. A moderate-to-tight finite element grid spacing could be selected to discern and analyze flow streamlines at the algae scale, for example on the order of ten algal cell diameters. The output of the CFD simulation will be the expected streamlines which show the path of fluid-driven cells into and out of light and dark regions and the photobioreactor. From these streamlines, the duration of light and dark exposure and the frequency with which the algae moves from light to dark exposure as it traverses the flow loop can be determined, and this illumination versus time relationship can be utilized in the above-described cell growth/photo modulation model to determine average growth rate around the flow loop.

If desired, experimental validation of the results of the CFD simulations can be performed using flow visualization studies of the actual flow trajectories in the photobioreactor. Such studies could be conducted by utilizing neutrally buoyant microspheres, simulating algal cells. In one particular embodiment, a laser can be configured and positioned to create a longitudinal sheet of coherent light through the active segment (i.e., conduit 102) of the photobioreactor. Such plane of laser illumination can be positioned to represent the boundary between “light” and “dark” regions. Its position can be adjusted to represent various expected light-dark transition depths within the conduit expected over the range of algal concentrations and illumination intensities that may be present during operation of the photobioreactor. In one embodiment, a combination of clear silica and fluorescent microspheres (available from Duke Scientific Corporation, Palo Alto, Calif.) could be used as model algae particles. The diameter and density of the microspheres should be selected to correspond to the particular strain of algae expected to be used in the photobioreactor. As the fluorescent microspheres cross the laser plane, they would scatter the laser beam and create a detectable “flash.” A video camera can be positioned to record such flashes, and the time between flashes can be used to measure the residence time of the particle in each of the two areas (i.e., the light and dark areas). A second laser plane could be generated, if desired, to visualize flow within an essentially perpendicular plane to the above longitudinal sheet, if it is desired to have a more detailed representation of the actual position of the various fluorescent microspheres within the cross section of the illuminated conduit.

Referring now to FIGS. 7a and 7b, two alternative computational and control methodologies for controlling and optimizing photomodulation in the photobioreactor of system 600 are described. The methodologies are similar and differ, primarily, in the computational parameters utilized for convergence (i.e. light/dark exposure intervals in the method of FIG. 7a, and predicted growth rate in the FIG. 7b method).

Referring now to FIG. 7a, in which one embodiment for creating and controlling photomodulation within a photobioreactor of a gas treatment system is disclosed. Initial step 702 is an optional model fitting step, which may be conducted off-line with a pilot-scale or micro-scale automated cell culture and testing system, as discussed above. Optional step 702 involves determining appropriate values of the various adjustable parameters comprising the constants of the growth rate/photomodulation mathematical model described above by fitting the model equations to experimental growth rate versus light/dark exposure interval data, as described above and in Wu and Merchuk, 2001.

In step 704, cell concentration within photobioreactor 100 is measured, for example through use of spectrophotometer 632. In step 706, the light intensity incident upon the active tube 102 of the photobioreactor is measured utilizing a light intensity measuring device (e.g., a light meter) 633. The measured cell concentration and illumination intensity can together be used to calculate, in step 708, the light penetration depth within tubular conduit 102 according to standard, well known methods (e.g., as described in Burlew, 1961).

In step 710, a mathematical calculation is performed to calculate, from the growth rate/photomodulation mathematical model, predicted light/dark exposure intervals (i.e., duration and frequency of light/dark exposure) required to yield a desired average growth rate, for example a maximal growth rate achievable (i.e. given the non-adjustable operating constraints of the system).

In step 712, computer implemented systems 602 performs a simulation (e.g., CFD simulation) of the liquid medium flow and determines the flow streamlines and patterns within the photobioreactor for a particular total gas flow rate and gas flow distribution to spargers 122 and 124. From the simulation, actual light/dark exposure intervals and photomodulation of the algae as it flows around the flow loop can be determined. The system can determine when algae within the liquid medium is exposed to light within active tube 102 by determining when it is within a region of the tube separated from the light exposed surface 132 by a distance not exceeding that which, as determined in the light penetration depth determination of step 708, would expose the algae to light at an intensity above that which is sufficient to drive photosynthesis (i.e., above that required to render the algae in the “active” photosynthetic mode as described in the above-discussed growth/photomodulation model). The precise light intensity, and corresponding penetration depth, required for active photosynthesis for a particular type or mixture of algae can be determined using routine experimental studies of algal growth versus light intensity in a model photobioreactor system.

In step 714, the light/dark exposure intervals and photomodulation characteristics determined in step 710 required to give a desired average growth rate are compared with the actual light/dark exposure intervals and photomodulation characteristics prevailing in the photobioreactor as determined in step 712. The simulation of step 712 is then repeated utilizing different gas flows and gas flow distributions until the difference between the exposure intervals determined in steps 710 and 712 is minimized and the simulations converge.

At this point, in step 716, computer implemented system 602 adjusts and controls the liquid flow rate within the photobioreactor and the liquid flow patterns (e.g., recirculation vortices) by, for example, adjusting the gas flow and gas distribution to spargers 122 and 124 so as to match the optimal values determined in step 714.

The alternative photomodulation determination and control methodology in FIG. 7b is similar to that disclosed in FIG. 7a, except that instead of the CFD and growth rate/photomodulation mathematical models converging upon calculated light/dark exposure intervals, the system is configured to run the simulations to determine flow parameters required to yield a desired predicted (i.e. by the growth rate/photomodulation model) growth rate.

Steps 702, 704, 706, 708, 712 and 716 can be performed essentially identically as described above in the context of the method outlined in FIG. 7a. In the current method, however, the actual light/dark exposure intervals and photomodulation data determined from the CFD simulation of step 712 is then utilized in step 710′ to calculate, utilizing the growth rate/photomodulation mathematical model, an average predicted growth rate that would result from such light/dark exposure characteristics. Step 712 is then repeated with different values of gas flow and gas distribution and a new predicted average growth rate is determined in step 710′. The computational procedure is configured to adjust the values in step 712 in order to converge in step 714′ upon a desired average growth rate as determined in step 710′, for example a maximum achievable growth rate. Once gas flow and gas distribution values resulting in such a predicted desired growth rate are determined, computer implemented control system 602 then applies these gas flow rates and distributions to the photobioreactor to induce the desired liquid flow dynamics in the system in step 716.

It should be appreciated that the above-described photomodulation control methodologies and systems can advantageously enable automated operation of the photobioreactor under conditions designed to create an optimal level of photomodulation. Advantageously, the system can be configured to continuously receive input from the various sensors and implement the methodologies described above so as to optimize photomodulation in essentially real time (i.e. with turn-around as fast as the computations can be performed by the system). This can enable the system to be quickly and robustly responsive to environmental condition changes that can change the nature and degree of photomodulation within the system. For example, in a particular embodiment and under one exemplary circumstance, computer implemented control system 602 could quickly and appropriately adjust the gas flow rates and distribution and, thereby, the liquid flow patterns and photomodulation within the photobioreactor, so as to account for transient changes in illumination, such as the transient passing of cloud cover, over a period of operation of the photobioreactor system.

The calculation methods, steps, simulations, algorithms, systems, and system elements described above may be implemented using a computer implemented system, such as the various embodiments of computer implemented systems described below. The methods, steps, systems, and system elements described above are not limited in their implementation to any specific computer system described herein, as many other different machines may be used.

The computer implemented system can be part of or coupled in operative association with a photobioreactor, and, in some embodiments, configured and/or programmed to control and adjust operational parameters of the photobioreactor as well as analyze and calculate values, as described above. In some embodiments, the computer implemented system can send and receive control signals to set and/or control operating parameters of the photobioreactor and, optionally, other system apparatus. In other embodiments, the computer implemented system can be separate from and/or remotely located with respect to the photobioreactor and may be configured to receive data from one or more remote photobioreactor apparatus via indirect and/or portable means, such as via portable electronic data storage devices, such as magnetic disks, or via communication over a computer network, such as the Internet or a local intranet.

Referring to FIG. 6a, computer implemented control system 602 may include several known components and circuitry, including a processing unit (i.e., processor), a memory system, input and output devices and interfaces (e.g., an interconnection mechanism), as well as other components, such as transport circuitry (e.g., one or more busses), a video and audio data input/output (I/O) subsystem, special-purpose hardware, as well as other components and circuitry, as described below in more detail. Further, the computer system may be a multi-processor computer system or may include multiple computers connected over a computer network.

The computer implemented control system may 602 include a processor, for example, a commercially available processor such as one of the series x86, Celeron and Pentium processors, available from Intel, similar devices from AMD and Cyrix, the 680×0 series microprocessors available from Motorola, and the PowerPC microprocessor from IBM. Many other processors are available, and the computer system is not limited to a particular processor.

A processor typically executes a program called an operating system, of which WindowsNT, Windows95 or 98, UNIX, Linux, DOS, VMS, MacOS and OS8 are examples, which controls the execution of other computer programs and provides scheduling, debugging, input/output control, accounting, compilation, storage assignment, data management and memory management, communication control and related services. The processor and operating system together define a computer platform for which application programs in high-level programming languages are written. The computer implemented control system 602 is not limited to a particular computer platform.

The computer implemented control system 602 may include a memory system, which typically includes a computer readable and writeable non-volatile recording medium, of which a magnetic disk, optical disk, a flash memory and tape are examples. Such a recording medium may be removable, for example, a floppy disk, read/write CD or memory stick, or may be permanent, for example, a hard drive.

Such a recording medium stores signals, typically in binary form (i.e., a form interpreted as a sequence of one and zeros). A disk (e.g., magnetic or optical) has a number of tracks, on which such signals may be stored, typically in binary form, i.e., a form interpreted as a sequence of ones and zeros. Such signals may define a software program, e.g., an application program, to be executed by the microprocessor, or information to be processed by the application program.

The memory system of the computer implemented control system 602 also may include an integrated circuit memory element, which typically is a volatile, random access memory such as a dynamic random access memory (DRAM) or static memory (SRAM). Typically, in operation, the processor causes programs and data to be read from the non-volatile recording medium into the integrated circuit memory element, which typically allows for faster access to the program instructions and data by the processor than does the non-volatile recording medium.

The processor generally manipulates the data within the integrated circuit memory element in accordance with the program instructions and then copies the manipulated data to the non-volatile recording medium after processing is completed. A variety of mechanisms are known for managing data movement between the non-volatile recording medium and the integrated circuit memory element, and the computer implemented control system 602 that implements the methods, steps, systems and system elements described in relation to FIGS. 6a, 7a, 7b, 8a, 8b, 8c, and 8d is not limited thereto. The computer implemented control system 602 is not limited to a particular memory system.

At least part of such a memory system described above may be used to store one or more data structures (e.g., look-up tables) or equations described above. For example, at least part of the non-volatile recording medium may store at least part of a database that includes one or more of such data structures. Such a database may be any of a variety of types of databases, for example, a file system including one or more flat-file data structures where data is organized into data units separated by delimiters, a relational database where data is organized into data units stored in tables, an object-oriented database where data is organized into data units stored as objects, another type of database, or any combination thereof.

The computer implemented control system 602 may include a video and audio data I/O subsystem. An audio portion of the subsystem may include an analog-to-digital (A/D) converter, which receives analog audio information and converts it to digital information. The digital information may be compressed using known compression systems for storage on the hard disk to use at another time. A typical video portion of the I/O subsystem may include a video image compressor/decompressor of which many are known in the art. Such compressor/decompressors convert analog video information into compressed digital information, and vice-versa. The compressed digital information may be stored on hard disk for use at a later time.

The computer implemented control system 602 may include one or more output devices. Example output devices include a cathode ray tube (CRT) display 603, liquid crystal displays (LCD) and other video output devices, printers, communication devices such as a modem or network interface, storage devices such as disk or tape, and audio output devices such as a speaker.

The computer implemented control system 602 also may include one or more input devices. Example input devices include a keyboard, keypad, track ball, mouse, pen and tablet, communication devices such as described above, and data input devices such as audio and video capture devices and sensors. The computer implemented control system 602 is not limited to the particular input or output devices described herein.

The computer implemented control system 602 may include specially programmed, special purpose hardware, for example, an application-specific integrated circuit (ASIC). Such special-purpose hardware may be configured to implement one or more of the methods, steps, simulations, algorithms, systems, and system elements described above.

The computer implemented control system 602 and components thereof may be programmable using any of a variety of one or more suitable computer programming languages. Such languages may include procedural programming languages, for example, C, Pascal, Fortran and BASIC, object-oriented languages, for example, C++, Java and Eiffel and other languages, such as a scripting language or even assembly language.

The methods, steps, simulations, algorithms, systems, and system elements may be implemented using any of a variety of suitable programming languages, including procedural programming languages, object-oriented programming languages, other languages and combinations thereof, which may be executed by such a computer system. Such methods, steps, simulations, algorithms, systems, and system elements can be implemented as separate modules of a computer program, or can be implemented individually as separate computer programs. Such modules and programs can be executed on separate computers.

The methods, steps, simulations, algorithms, systems, and system elements described above may be implemented in software, hardware or firmware, or any combination of the three, as part of the computer implemented control system described above or as an independent component.

Such methods, steps, simulations, algorithms, systems, and system elements, either individually or in combination, may be implemented as a computer program product tangibly embodied as computer-readable signals on a computer-readable medium, for example, a non-volatile recording medium, an integrated circuit memory element, or a combination thereof. For each such method, step, simulation, algorithm, system, or system element, such a computer program product may comprise computer-readable signals tangibly embodied on the computer-readable medium that define instructions, for example, as part of one or more programs, that, as a result of being executed by a computer, instruct the computer to perform the method, step, simulation, algorithm, system, or system element.

In another set of embodiments, the invention also provides methods for pre-adapting and pre-conditioning algae or other photosynthetic organisms to specific environmental and operating conditions expected to be experienced in a full scale photobioreactor during use. As mentioned above, the productivity and long-term reliability of algae utilized in a photobioreactor system for removing CO₂, NO_{X} and/or other pollutant components from a gas stream can be enhanced by utilizing algal strains and species that are native or otherwise well suited to conditions and localities in which the photobioreactor system will be utilized.

As is known in the art (see, for example, Morita, M., Y. Watanabe, and H. Saiki, “Instruction of Microalgal Biomass Production for Practically Higher Photosynthetic Performance Using a Photobioreactor.” Trans IchemE. Vol 79, Part C, September 2001.), algal cultures that have been exposed to and allowed to proliferate under certain sets of conditions can become better adapted and suited for long term growth and productivity under similar conditions. The present invention provides methods for reproducibly and predictably pre-conditioning and pre-adapting algal cultures to increase their long term viability and productivity under a particular expected set of operating conditions and to prevent photobioreactors inoculated with such algal species from having other, undesirable algal strains contaminating and dominating the algal culture in the photobioreactor over time.

In many current photobioreactor systems, chosen, desirable strains of algae can be difficult to maintain in a photobioreactor that is not scrupulously sterilized and maintained in a condition that is sealed from the external environment. The reason for this is that the algal strains being utilized in such photobioreactors are not well adapted or optimized for the conditions of use, and other, endemic algal strains in the atmosphere are more suitably conditioned for the local environment, such that if they have the ability to contaminate the photobioreactor they will tend to predominate and eventually displace the desired algae species. Such phenomena can be mitigated and/or eliminated by using the inventive adaptation protocols and algal cultures by practicing such protocols described below. Use of such protocols and algae strains produced by such protocols can not only increase productivity and longevity of algal cultures in real photobioreactor systems, thereby reducing capital and operating costs, but also can reduce operating costs by eliminating the need to sterilize and environmentally isolate the photobioreactor system prior to and during operation, respectively.

Typically, commercially available algal cultures are adapted to be grown under ordinary laboratory conditions. Accordingly, such commercially available algal cultures are typically not able or well-suited to be grown under one or more conditions of light exposure, gas composition, temperature fluctuation, etc. to which algae would be expected to be exposed in the field in a gas-treatment photobioreactor system, such as described above. For example, most commercially available algal cultures are conditioned for growth at relatively low light levels, such as 150 micro Einstein per meter squared per second (150 μEm⁻²s⁻¹). Exposure of such cultures to sunlight in photobioreactor gas-treatment systems of the invention—which may expose the organisms to light intensities of 2,500 μm⁻²s⁻¹ or greater—will typically cause substantial photoinhibition rendering such cultures unable to survive and/or grow adequately, and, therefore, unable to successfully compete with deleterious native species that may infiltrate the photobioreactor. Accordingly, as described in more detail below, one aspect of the inventive adaptation processes is to precondition and adapt such commercially available laboratory cultures to light of an intensity and duration expected to be experienced in full-scale photobioreactors of the invention.

In addition, as described above, the inventive photobioreactors, in certain embodiments, may be configured and operated to subject the algae to relatively high frequency photomodulation cycles. While such high-frequency photomodulation can be beneficial for the grown of the algae, unadapted and unconditioned algal strains may not be well adapted to and ideally suited for growing under such conditions. Accordingly, in certain embodiments, the inventive adaptation methods are able to produce algal strains that are adapted to and well-suited for growing under conditions of high-frequency photomodulation (e.g., light/dark interval switching frequencies of one per minute, one per second, one per 1/10 second, one per 1/100 second, one per millisecond, or higher). Similarly, many components found in typical flue gases, which are desirably removed by the photobioreactors of the current invention in certain embodiments, may be lethally toxic to and/or can substantially inhibit growth of nonadapted algal strains at concentrations that may be found in flue gas. For example, the concentration of CO₂, NO_{X}, SO_{X}, and heavy metals such as Hg in flue gases may be substantially higher than those that are toxic or deleterious to many unadapted algal strains.

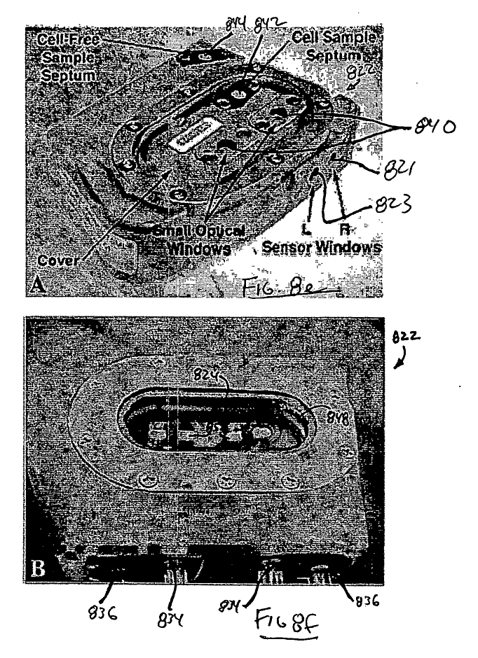

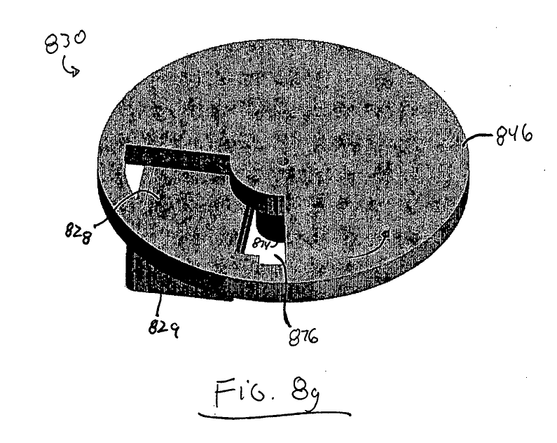

Certain exemplary embodiments of such algal adaptation and pre-conditioning methods are illustrated in FIGS. 8a-8d. Referring to FIG. 8a, initially, in step 802, one or more algae species are selected which are expected to be at least compatible with, and preferably well suited for, the expected environmental conditions at the particular photobioreactor installation site. In step 804, in a pilot-scale or a micro-scale photobioreactor system, an algal culture comprising the algae species from step 802 is exposed to a set of defined environmental, medium, growth, etc. conditions that are specifically selected to simulate conditions to which the algae will be exposed in the photobioreactor during operation, e.g., as part of a gas treatment system. In step 806, the algal cultures are grown and propagated under the selected simulation conditions for a sufficient period of time to allow for multi-generational natural selection and adaptation to occur. Depending on the algal species, this period may be anywhere from a few days to a few weeks to as much as a few months. At the end of adaptation, the adapted algae is harvested in step 808 and provided to an operator of a photobioreactor system, so that the photobioreactor may be inoculated with the algae to seed the photobioreactor.

In certain embodiments, steps 804 and 806 illustrated in FIG. 8a, which together comprise adaptation step 807, are performed according to a protocol such as that illustrated in FIG. 8b. Referring to FIG. 8b, after the selecting step 802, a pilot or small-scale photobioreactor, such as those described in more detail below, is inoculated in step 807a with an unadapted (starter) algal culture. Then, initially, in step 807b, the culture is grown under conditions that are known to facilitate normal growth for the particular algal culture until the culture is fully established and growing well. Then, in step 807c, gradually, for example over a period of time equal to many doubling times of the algal culture (i.e., many generations of growth) the initial conditions are adjusted to a set of defined growth conditions that are selected to simulate conditions to which the algae will be exposed in a full-scale photobioreactor of a gas treatment system.

In certain embodiments, in step 807c, the rate and amount of adjustment of particular growth conditions is selected to be gradual enough to permit the culture to continue to grow during the entirety of the adaptation process. In certain embodiments, changes may occur for one or a few process conditions at a time, so that the algal culture becomes adapted to one or a subset of defined growth conditions simulating operating conditions in the gas treatment system before being adapted to others (i.e., the adaptation to particular growth conditions occurs non-simultaneously). In other embodiments, each of the growth conditions that are different for the defined set of growth conditions simulating actual operating conditions of the photobioreactor, as compared to the initial growth conditions of step 807b, are gradually adjusted simultaneously over the selected period of time. As mentioned above, in preferred embodiments, the gradual adjustment of growth conditions in step 807c occurs over many generations and doubling times of the culture, and, at least, should exceed one doubling time of the starter culture. For example, in certain embodiments, the overall length of the period over which growth conditions are adjusted in step 807c can exceed two doubling times, five doubling times, ten doubling times, 100 doubling times, 200 doubling times, or 500 doubling times of the starter culture grown under conditions as outlined in step 807b.

As discussed above, and as illustrated and discussed below in the context of FIG. 8c, the gradual adjustment step 807c may be effected to facilitate adjustment of initial growth conditions to the defined growth conditions simulating photobioreactor gas-treatment system operation in a variety of ways. The particular manner and sequence of adjustment may vary substantially depending upon the particular nature, sensitivity, adaptability, etc., of the starter culture and the particular algal strains chosen. Those of ordinary skill in the art, given the teachings and information provided herein, can readily determine a suitable or optimal course of gradual parameter adjustment to effect a desirable level of adaptation of any selected algal strain/culture using no more than ordinary skill and routine experimentation and optimization.

FIG. 8c illustrates certain exemplary embodiments for performing step 807c of FIG. 8b. Referring to FIG. 8c, a gradual parameter adjustment protocol is outlined that entails changing parameter values, either simultaneously or sequentially, or a combination thereof, over the adjustment period in a series of small increments. In certain embodiments, the increments may be evenly spaced and/or of equal magnitude. In alternative embodiments, depending on the particular parameters being adjusted and their effect on the growth of culture, the increments may be unequally spaced over the entire interval and/or be of unequal magnitude at different intervals over the period.

In step 807ci, the value of at least one growth parameter is changed by an increment that is selected to be small enough to still permit survival and growth of the culture after the change. In one embodiment, represented by step 807cii′, the culture is then allowed to equilibrate and adjust to the new condition over a fixed interval of time selected to be sufficient to permit the growth rate to stabilize and recover. For example, such fixed interval of time may be at least two doubling times of the starter culture under the initial conditions, or greater. In other embodiments, especially for those in which the pilot/small-scale photobioreactor system utilized for adaptation includes the capability of automated growth rate determination of the culture, adjustment can be made as described in step 807cii″. In such embodiment, after incrementally changing the value of the growth parameter, the culture is allowed to equilibrate and adjust to the new growth condition until a measured growth rate is determined to reach a stable plateau, before performing a subsequent incremental change. After waiting the requisite period of time described in step 807cii′ or 807cii″, another incremental change to the same and/or different growth parameter is made, and the process is repeated until the growth parameters have been completely adjusted to the defined growth conditions selected to simulate conditions to which the algae will be exposed in the photobioreactor of the gas treatment system (step 807ciii). At this point, the adapted algal cultures can be continued to be cultured at the defined growth conditions for a period of time selected to be great enough to allow the growth rate to stabilize and to permit the cultures to become optimally suited to the defined simulation conditions. Typically, the adapted culture will be grown and maintained at the defined growth conditions indefinitely and until some sample of the adapted algae is harvested for inoculation into a photobioreactor of a gas-treatment system (steps 808 and 810 of FIG. 8a).

Referring again to FIG. 8c, after the adaptation process is complete, the effectiveness of the adaptation process can be determined in step 807civ by comparing the growth rate of the adapted algae to that of an equivalent unadapted culture (e.g., a sample of starter culture from step 807a of FIG. 8b) at the defined set of growth conditions selected to simulate conditions of operation of a photobioreactor in a gas-treatment system. In certain embodiments, the culture, when adapted, is able to grow under the defined set of conditions with a doubling time that is no greater than 50% that of an unadapted sample (i.e., twice the growth rate). In certain embodiments, the culture, when adapted, may be able to grow at the defined set of conditions with a doubling time that is no greater than 33%, 30%, 25%, 20%, 15%, 10% or less that of an unadapted sample of the starter culture subjected to the defined set of conditions.

As mentioned above, one growth parameter that may be very different in the photobioreactors of a gas-treatment systems of the invention during operation from that to which typical, commercially-available algal cultures are accustomed is light exposure, i.e., intensity and photomodulation frequency. For example, illuminance (or photon flux density) in full sunlight, such as may be experienced by cultures growing in photobioreactors that are part of gas-treatment systems of the invention, can be 2500 μEm⁻²s⁻¹ or more. Typical laboratory prepared cultures of algae are typically grown under conditions of much lower light intensity, e.g., 150 μEm⁻²s⁻¹ or less. In such commercially available cultures, a reduction in the growth rate of such cultures via photoinhibition may occur, depending on the particular algal species, at levels of about, for example, 300 μm⁻²s⁻¹. Accordingly, such commercially available cultures are poorly suited for, and may experience high levels of photoinhibition and poor growth or cell death, under conditions expected to be experienced by algal cultures in operation in the inventive photobioreactor of gas-treatment systems. Additionally, as mentioned above, commercially-available algal cultures may not be accustomed to photomodulation at high frequency.

In order to adapt algal cultures to higher illumination intensities, such as those that may be experienced in the inventive photobioreactors in full sunlight, in certain embodiments, prior to initiating photomodulation, a starter culture is gradually adapted, as described in FIGS. 8a-8c above, to illumination intensities that are above the intensity that is known to be capable of causing a reduction in the growth rate of the starter culture via photoinhibition. “Known to be capable of causing reduction in the growth rate of the starter culture via a photoinhibition” refers herein to such an intensity being known for unadapted cultures/samples either through values available in the published literature for such cultures or through routine screening tests to define a photoinhibition threshold. Once the culture has become adapted to growth at a light intensity above the known photoinhibition threshold, then, as described in more detail below, in the presently described embodiment, adaptation to higher frequency photomodulation may be commenced. In certain embodiments, the algal culture may be adapted to the light intensity that is at least twice that known to be capable of causing a reduction in the growth rate of an unadapted starter sample of the culture, in other embodiments the intensity level to which the culture is adapted may be 3, 5, 10, 20, or more times that known to be capable of causing growth rate reduction via photoinhibition of the starter sample.

In certain embodiments, the algal culture is adapted to relatively high-frequency photomodulation cycles, simulating those that may be expected during operation of a photobioreactor in a gas-treatment system of the invention. A photomodulation cycle comprises a period of illumination at an intensity above a threshold able to drive photosynthesis in the culture and a period of exposure to a lower intensity below the threshold capable of driving photosynthesis of the organisms of the culture. The frequency of the cycle can be characterized by the number of transitions from high (light) to low (dark) illumination intensities per unit of time. In certain embodiments, the duration of light intervals and dark intervals over a given light/dark cycle may be the same or, in other embodiments, the light period may exceed the dark period or the dark period may exceed the light period. Accordingly, it is possible to adapt the algae to both photomodulation frequency and relative duration of light versus dark periods within a given light/dark cycle, according to the methods of the invention. In certain embodiments, the algal culture may be adapted and preconditioned for growth conditions that comprise a variation in light intensity to cause photomodulation at a light/dark cycling frequency of at least one light/dark transition per minute. In other embodiments, the algal culture may be conditioned for light/dark cycling frequencies of at least one light/dark transition per 30 seconds, per 10 seconds, per 5 seconds, per second, per ½ second, per 1/10 second, per 1/100 second, per millisecond, or greater.

In certain embodiments, it may be desirable to develop a preconditioned, adapted algae, according to the methods of the invention, that is preconditioned and adapted to grow and thrive under conditions of exposure to one or more typical pollutant gases, dissolved in the growth medium, that may be found in flue gas or other gases being treated by a gas treatment system in which the algal culture is intended to be used. In certain such embodiments, it may be desirable to adapt an algal culture to growth in a liquid medium that contains at least one of dissolved CO₂, NOₓ, SOₓ, and/or heavy metals, such as Hg. In certain embodiments, the algal culture is adapted to concentrations of such gases dissolved in the liquid medium that are typical of those that would be experienced when the algal culture is contained within a photobioreactor of a gas-treatment system of the invention that is fed a gas for treatment containing one or more of the above pollutant gases at concentrations typically found in flue gas, or other combustion gases that may be treated. Accordingly, in certain embodiments, an algal culture may be exposed to and adapted to a defined set of growth conditions that comprises growth of a culture in a liquid medium, wherein the liquid medium has been exposed in mass transfer communication with at least one of the above-mentioned substances.

A liquid medium that is exposed in “mass transfer communication” with a gas comprising at least one of the above-mentioned substances refers to such liquid medium being placed either in direct interfacial contact with such gas (e.g., as when the gas is sparged or bubbled into the liquid) or to the liquid medium being separated from the gas by a liquid impermeable membrane or layer through which one or more components of the gas or gas mixture is able to diffuse over a time scale allowing the dissolution of at least some of such diffusible components into the liquid medium. In certain embodiments, the liquid medium may be exposed in mass transfer communication with a gas under conditions sufficient to allow dissolution of soluble gas components in the liquid at amounts indicative of mass transfer equilibrium having been reached between the gas and the liquid at ambient conditions of the environment in which the mass transfer communication occurred (e.g. about 25° C. and atmospheric pressure at sea level in certain embodiments). In certain such embodiments, the gas to which the liquid medium is exposed in mass transfer communication can comprise an actual flue gas or a gas mixture simulating flue gas. In certain embodiments, the gas comprises at least about 5% wt CO₂, and in certain embodiments between about 8% wt CO₂ and about 15% wt CO₂. In certain embodiments, the gas comprises NOₓ in an amount of at least 1 ppm, in certain embodiments at least about 10 ppm, in certain embodiments at least about 100 ppm, and in certain embodiments between about 100 ppm and about 500 ppm. In certain embodiments, the gas comprises SOₓ in an amount of at least about 1 ppm, in other embodiments at least about 50 ppm, in other embodiments between about 50 ppm and about 1,000 ppm, and in other embodiments at least about 1,000 ppm.

While the presently disclosed adaptation methods are particularly well suited for adapting and preconditioning algal species to define growth conditions that are selected to simulate conditions in a photobioreactor of a gas treatment system of the invention, in other embodiments, other photosynthetic organisms, for example euglena may be similarly adapted and preconditioned. While essentially any algal species, species of other photosynthetic organisms, or collection of such species can potentially be adapted and preconditioned according to the methods disclosed herein, in certain embodiments, a preconditioned culture produced according to the invention will comprise at least one species of algae selected from the genuses Chlorella, Spirolina, Chlamydomonas, Dunaliella, and/or Porphyridium. In certain exemplary embodiments, a preconditioned culture produced according to the invention comprises at least one of Dunaliella tertiolecta, Porphyridium sp., Dunaliellaparva, Chlorellapyrenoidosa, and/or Chlamydomonas reinhardtii.

In certain embodiments, the pilot-scale photobioreactor utilized in adaptation step 807 could be similar to or identical to those described above in the context of determining growth model constants for the growth photomodulation mathematical model above. For example, a small volume, thin-film tubular photobioreactor as described in Wu and Merchuk, 2001 could be utilized.

In certain embodiments, step 807 is carried out and performed utilizing an existing or custom-developed automated cell culture and testing system, in certain embodiments utilizing a plurality of precisely controllable small-scale bioreactors, which can be operated as photobioreactors, thus allowing for precise, simultaneous multi-parameter manipulation and optimization of algal cultures with the system. An “automated cell culture and testing system” as used herein, refers to a device or apparatus providing at least one bioreactor and which provides the ability to control and monitor at least one, and preferably a plurality of, environmental and operating parameters. Certain embodiments employ systems that are automated cell culture and testing systems having at least one, and more preferably a plurality of, bioreactors providing photobioreactors having a culture volume of between about 1 microliter and about 1 liter, between about 0.5 ml and about 100 ml, or between about 1 ml and about 50 ml. Potentially suitable, as provided or after suitable modifications, automated cell culture and testing systems are available and are described, for example, in (Vunjak-Novakovic, G., de Luis J., Searby N., Freed L. E. Microgravity Studies of Cells and Tissues. Ann. NY Academy of Sciences; Vol. 974, pp. 504-517 (2002); Searby N. D., J. Vandendriesche, L. Sun, L. Kundakovic, C. Preda, I. Berzin and G. Vunjak-Novakovic (2001) Space Life Support From the Cellular Perspective, ICES Proceeding 0011ICES-331 (2001); de Luis, J., Vunjak-Novakovic, G., and Searby N. D. Design and Testing of the ISS Cell Culture Unit. Proc. 51^{st} Congress of the Astronautical Federation, Rio de Janeiro, Oct. 2-6, 2000; Searby N. D., de Luis, J., and Vunjak-Novakovic, G. Design and Development of a Space Station Cell Culture Unit. J. Aerospace, Vol. 107, pp. 445-457 (1998); and U.S. Pat. No. 5,424,209; U.S. Pat. No. 5,612,188; U.S. Patent Application Publication 2003/0040104; U.S. Patent Application 2002/0146817; and International Application Publication no. WO 01/68257, each of the above patents, published applications, and literature references being incorporated herein by reference).

In certain configurations, such an automated cell culture and testing system includes computer process control and monitoring enabling growth conditions such as temperature, light exposure intervals and frequency, nutrient levels, nutrient flow and mixing, etc. to be monitored and adjusted. Certain embodiments can also provide on-line video microscopy and automatic sampling capability. Such automated cell culture and testing systems can allow multidimensional adaptation and optimization of the algal system by enabling control of a variety of growth parameters, autonomously.

In one particular embodiment, an automated cell culture and testing system, as described above, is configured to expose the algal cultures to expected conditions of: liquid medium composition; liquid medium temperature; liquid medium temperature fluctuation magnitude, frequency and interval; pH; pH fluctuation; light intensity; light intensity variation; light and dark exposure durations and light/dark transition frequency and pattern; feed gas composition; feed gas composition fluctuation; feed gas temperature; feed gas temperature fluctuation; and others; and to carry out the above-described culture adaptation protocols.

In one exemplary embodiment, high frequency light/dark cycles simulating photomodulation created by turbulent eddies and/or recirculation vortices in a light exposed part of the photobioreactor are simulated utilizing a light source shining on a micro-photobioreactor of an automated cell culture and testing system through a variable-speed chopper wheel with interchangeable disks machined with slits, or otherwise provided with opaque and transparent regions, to give appropriate frequencies of photomodulation and ratio of light/dark periods. In one example, photomodulation light/dark interval frequencies of 0.1, 0.5, 1, 10, 100, and 1000 cycles per second are simulated. As described above, each adaptation step 807 should occur over a long enough period to allow for multi-generational adaptation. In a particular embodiment in which an algae species of Dunaliella is pre-adapted, each adaptation increment (FIG. 8c) is allowed to occur over at least a 1-, 2-, or 3-day cycle to allow a multi-generational adaptation.

FIGS. 8d-8g illustrate various components of an exemplary embodiment of an automated cell culture and testing system that can be utilized to perform the above-described cell culture adaptation and preconditioning methods. It should be emphasized that the particular example of a cell culture system illustrated in FIG. 8d comprises only one of a very wide variety of possible configurations and set ups. As would be understood by those of ordinary skill of the art, a wide variety of perfusion and non-perfusion based cell culture systems, including small-scale cell culture systems, can potentially be adapted to be used within the context of the invention. Accordingly, the particular system and components described herein are purely exemplary and may be otherwise configured, substituted, or eliminated in other embodiments within the scope of the invention defined by the claims appended below. The exemplary embodiment illustrated in FIGS. 8d-8g comprises a modified and adapted cell culture system similar to that described in: Vunjak-Novakovic, G., de Luis J., Searby N., Freed L. E. Microgravity Studies of Cells and Tissues. Ann. NY Academy of Sciences; Vol. 974, pp. 504-517 (2002); Searby N. D., J. Vandendriesche, L. Sun, L. Kundakovic, C. Preda, I. Berzin and G. Vunjak-Novakovic (2001) Space Life Support From the Cellular Perspective, ICES Proceeding 011CES-331 (2001); de Luis, J., Vunjak-Novakovic, G., and Searby N. D. Design and Testing of the ISS Cell Culture Unit. Proc. 51^{st} Congress of the Astronautical Federation, Rio de Janeiro, Oct. 2-6, 2000; Searby N. D., de Luis, J., and Vunjak-Novakovic, G. Design and Development of a Space Station Cell Culture Unit. J Aerospace, Vol. 107, pp. 445-457 (1998), to which the readers refer for additional details.

Referring to FIG. 8d an automated cell culture and testing system 820 is schematically illustrated comprising a perfusion-based cell culture system including a cell culture module 822 including therein a cell cultured chamber 824 and medium containing cell-free region 826. The configuration of cell culture module 822 is described in more detail in the above-mentioned references and is illustrated in greater detail in FIGS. 8e and 8f. In certain embodiments, the cell culture module 822 comprises a small-scale bioreactor having an internal volume between about 1 micro liter, in certain embodiments between about 0.5 ml and about 50 ml, and in certain embodiments between about 1 ml and about 10 ml. As is described in more detail below, automated cell culture and testing system 820 further comprises an adjustable source of artificial light 828 capable of driving photosynthesis and a light source modulator 830 that is constructed and arranged to vary the intensity of the light that reaches the algal cells 832 in cell culture chamber 824 between a first (light) intensity and a second (dark) intensity, preferably at a frequency of at least one variation per second, and in certain embodiments at frequencies mentioned above with regard to adaptation to defined levels of photomodulation simulating actual conditions of photobioreactors of the gas treatment systems of the invention.

In the illustrated exemplary embodiment, cell culture system 820 is configured as a perfusion-based system, and cell culture module 822 includes at least one liquid medium inlet 834 and at least one liquid medium outlet 836 interconnected in a flow loop described in more detail below, whereby liquid medium is continuously or intermittently removed from cell culture module 822, treated to effect maintenance or variation of various cell culture parameters, and returned to cell culture module 822. In alternative embodiments, cell culture module 822 and cell culture system 820 may be configured as a non-perfusion system in which adjustments in various cell culture parameters are effected upon the liquid medium while it remains contained in the cell culture module. Such non-perfusion systems are well know and may be substituted for the perfusion-based system illustrated and described herein.

Automated cell culture system 820 includes, in certain embodiments, a plurality of different sensors, actuators, valves, flow meters, etc., for measuring, maintaining, and/or adjusting/changing various cell culture parameters to provide defined growth conditions in order to effect various culture adaptation protocols according to the invention. Such components may comprise a variety of sensors, flow meters, etc., similar to those described above in the context of FIG. 6a, and the system can further comprise a computer implemented control system 602, that can be essentially the same as or similar to that described above in the context of FIG. 6a. In certain embodiments, wherein the cell culture module 822 comprises a small-scale bioreactor, sensors provided to monitor liquid medium conditions within cell culture module 822, for example pH sensor 614, CO₂ sensor 821, and oxygen sensor 823, may be configured as optical chemical sensors (e.g. such as those based on fluorescence modulation), which are well known in the art as being particularly well suited for non-invasive parameter measurement of small volume systems (see, e.g., U.S. Pat. Nos. 6,673,532; 6,285,807; 6,051,437; 5,628,311; 5,606,170; and 4,577,110, each incorporated herein by reference).

In the system illustrated FIG. 8d, the interior of cell culture module 822 is partitioned by an optional cell retaining membrane(s) 838, which divide the interior of cell culture module 822 into a cell culture chamber 824, including suspended algae 832, and cell-free volume 826 containing liquid medium. Membranes 838 can be formed of any of a wide variety of biocompatible materials, which are well known to those of ordinary skill in the art, and preferably have a permeability and pore size selected to allow the liquid medium and components dissolved therein to permeate freely through the membranes while retaining in cell culture chamber 824 algal cells 832. In alternative embodiments, in which it is not unacceptable or deleterious to circulate cells around the profusion loop of the cell culture system, membranes 838 may be eliminated.

Cell culture module 822, as illustrated, further includes a top surface having two small optically transparent windows 840 therein providing visual access to culture chamber 824, for example, to allow visual observation, video monitoring, illumination of the culture chamber, etc. In addition, cell culture module 822 includes a cell sampling septum 842 and a cell-free sample septum 844 to facilitate the ability to insert and withdraw samples to and from cell culture chamber 824 and cell-free volume 826, in certain embodiments in a sterile manner, respectively. Cell sampling septum 842 may also be used to remove cells from culture chamber 824 for the purpose of diluting the culture with cell free-medium when cell density exceeds a certain value. Such dilution/subculturing may be performed manually or automatically by an automated sampling station (not shown) under the control of computer implemented control system 602.

The bottom surface of cell culture module 822, which is positioned in spaced-apart relationship from light cutter wheel 846 of light source modulator 830 and light source 828, includes a region 848 comprising an optical window that is at least partially transparent to light of a wavelength capable of driving photosynthesis. As explained in greater detail below, in the illustrated embodiment, light source 828 is configured and positioned to direct light 850 so that it is incident upon transparent region 848 of cell culture module 822, thereby permitting the light to entered cell culture chamber 824 to illuminate the culture and drive photosynthesis and growth. In certain embodiments, light source 828 comprises a full-spectrum illuminator, which has an intensity that can be adjusted by, for example, modulating the power to the light source (e.g. under the control of computer implemented system 602), varying the distance from the light source to the optically transparent region 848 of the cell culture module 822, etc. In certain embodiments, light source 828 can comprise one or more incandescent lamps, fluorescent lamps, LEDs, lasers, or other known light source. In certain embodiments, other than that illustrated in FIG. 8d, cell culture module 822 may not include an optically transparent region 848 but, rather, may include a light source that is located directly within culture chamber 824. In certain such embodiments, and/or in alternative embodiments having a light source 828 positioned externally of culture chamber 824, that utilizes a light source modulator not including the illustrated cutter wheel mechanism 846 for high frequency modulation of light intensity and provision of photo modulation, high frequency photo modulation could be effected by for example, controllable rapid on/off switching of the power supply 829 to light source 828, for example, with an electric pulse generator, strobe circuit, etc.

In certain embodiments, in order to ensure that the contents of culture chamber 824 are well mixed so that algal cells 832 contained within the culture chamber are exposed to essentially uniform light intensity throughout the chamber (i.e. to reduce the effects of any photo modulation due to flow patterns within culture chamber 824), culture chamber 824 can include therein one or more magnetic stirring devices such as magnetic stir bars 852 that can be driven in rotation by a stir bar motor 854. In addition, it may be desirable to configure cell culture module 822 so that it has a thickness (T) that is small enough to ensure that algae cell located it any vertical position within culture chamber 822 are subjected to a light intensity that is substantially similar to cells located in any other position within the culture chamber.

As illustrated, automated cell culture system 820 includes a single cell culture module 822 and perfusion loop 856 associated therewith. However, in certain embodiments, cell culture system 820 may be made part of a larger, multi-module, automated cell culture system comprising a plurality of cell culture modules and associated perfusion loops configured in parallel. Such a multi-module system could permit simultaneous adaptation of multiple algal cultures to a plurality of different sets of defined culture parameters.

Perfusion loop 856, in certain embodiments, comprises flexible tubing 858 for medium recirculation, which has low gas permeability. A variety of suitable materials for forming such tubing are well known to those of ordinary skill in art and include, for example, polymeric tubing made out of one or more suitable polymers such as, for example, poly(vinyl chloride), polyethylene, polypropylene, etc. A pump 860, for example a peristaltic pump, may be used for circulation and may be controlled via computer implemented system 602 to provide a desirable liquid medium flow rate, for example as measured by flow meter 624. In certain embodiments, the computer implemented control system 602 can be provided with the capability to, provide periodic flow, provide for reverse flow, unsteady flow, etc.

Perfusion loop 856 can further comprise a gas exchanger 862 that is constructed and arranged to provide mass transfer communication between the liquid medium and gas comprising at least one component dissolvable in the liquid medium. In the illustrated embodiment, gas exchanger 862 comprises a silicone-coil gas exchanger in which liquid medium passes through a selected length of coiled silicone tubing 863, having high permeability for one or more dissolvable gas species, such as O₂, CO₂, NOₓ, SOₓ, etc. As would be understood by those of ordinary skill in the art, the particular degree of gas permeation and mass transfer into the liquid medium in gas exchanger 862 depends upon a variety of design factors well known to those of ordinary skill in the chemical engineering arts; such as, for example, the permeability of tubing 864 for the particular species, the length of tubing 863, the flow rate of liquid medium through the tubing, the temperature, the pressure of gas within gas exchanger 862, the composition and concentration of dissolvable components within the gas within gas exchanger 862, etc. Appropriate values of the above parameters that can provide a desirable level of mass transfer and dissolution of dissolvable gas species in the liquid medium for a given pass through gas exchanger 862 can be readily determined by those of ordinary skill in the chemical engineering arts. Gas exchanger 862 is connected in fluid communication with a gas source 866, which can comprise, in certain embodiments, flue gas or a gas mixture simulating flue gas and/or a defined gas mixture containing one or more components dissolvable in the liquid medium to which exposure it is desired to adapt algal cells 832. Such components and there concentrations have been discussed previously in the context of the inventive culture adaptation protocols.

In alternative embodiments, the silicone-coil gas exchanger 862 illustrated may be supplemented or replaced by a wide variety of other gas exchangers of known design. For example, in certain embodiments, the gas exchanger could comprise a stacked membrane or hollow fiber membrane type gas exchanger. In yet other embodiments, the gas exchanger could comprise a vessel containing the liquid medium into which gas is sparged, similar to the gas exchange systems utilized in photobioreactor apparatus 100 illustrated and discussed previously. In yet other embodiments, especially in embodiments wherein the cell culture system is a non perfusion-based system not comprising a perfusion loop, a gas exchanger could comprise one or more external surfaces of such cell culture module being formed of a gas permeable, liquid impermeable membrane. In such an embodiment, the entire cell culture module could be contained within an enclosure providing a surrounding gaseous environment comprising a gas including one or more components dissolvable in the liquid media that are desired to be added to the liquid media for adaptation of the cell culture.

As illustrated, perfusion loop 856 of automated cell culture system 820 further includes a liquid medium reservoir 868 connected in liquid communication with one or more sources 870, of fresh medium or other additives for adjustment of the composition of the liquid medium in cell culture module 822. Cell culture medium reservoir 868 may also comprise a medium outlet 872 from which spent medium may be removed, samples extracted, etc.

Light source modulator 830 in the embodiment illustrated in FIG. 8d comprises a rotating cutter wheel 846 (see FIG. 8g) driven in rotation by a variable speed motor 874, which is controlled by computer implemented system 602. Cutter wheel 846 can be made from a material that is optically opaque to light of a wavelength capable of driving photosynthesis and can include in spaced apart location(s) at one or more angular positions on the disk optically transparent region(s) 876, which are at least partially transparent to light of wavelength capable of driving photosynthesis (see FIG. 8). In one embodiment, cutter disk 846 is formed of an opaque medal having a plurality of slits therein comprising transparent regions 876. In other embodiments, cutter disk 846 could be made of an opaque material not having slits therein, but rather having regions of the material that have been rendered transparent to light of a wavelength capable driving photosynthesis. In alternative embodiments, cutter disk 846 can be made of a material that is transparent to light of a wavelength capable of driving photosynthesis and made to include thereon regions comprising an opaque coating, dye, etc. to provide an essentially equivalent effect as the illustrated cutter disk 846. In certain embodiments, transparent regions 876 of cutter disk 846 need not be completely transparent to light of a wavelength capable of driving photosynthesis, but, rather, could comprise regions of partial transparency and/or could comprise wavelength-selective optical filters, polarizers, etc. The light/dark cycle frequency and light and dark time interval duration can be controlled, in certain embodiments, via either or both of: (1) the number, position, and size of optically transparent region(s) 876 on the cutter wheel, and (2) the rotational speed of the cutter wheel, which is dictated by variable speed motor 874.

FIG. 9 illustrates one embodiment of an integrated system for performing an integrated combustion method, wherein combustion gases are treated with a photobioreactor system to mitigate pollutants and to produce biomass, for example in the form of harvested algae, with the bioreactor system, which can be utilized as a fuel for the combustion device and/or for the production of other fuel products, such as hydrogen, as is illustrated in FIG. 10 and described below. Integrated system 900 can be advantageously utilized to both reduce the level of pollutants emitted from a combustion facility into the atmosphere and, in certain embodiments, to reduce the amount of fossil fuels, such as coal, oil, natural gas, etc., burned by the facility and/or to produce a non-fossil, clean fuel, such as hydrogen, with and/or from the biomass. Such a system can potentially be advantageously utilized for treating gases emitted by facilities such as fossil fuel (e.g., coal, oil, and natural gas)— fired power plants, industrial incineration facilities, industrial furnaces and heaters, internal combustion engines, etc. Integrated gas treatment/biomass-producing system 900 can, in certain embodiments, substantially reduce the overall fossil fuel requirements of a combustion facility, while, at the same time, substantially reducing the amount of CO₂ and/or NO_{X} released as an environmental pollutant, and, in certain embodiments providing biomass useful in producing clean fuel products, such as hydrogen.

Integrated system 900 includes one or more photobioreactors or photobioreactor arrays 902, 904, and 906. In certain embodiments, these photobioreactors can be similar or identical in design and configuration to those previously-described in FIGS. 1, 2, and 6a or in FIGS. 3 and 3a. In alternative embodiments, other embodiments of the inventive photobioreactors could be utilized or conventional photobioreactors could be utilized. Except for embodiments wherein system 900 utilizes photobioreactors provided according to the present invention (in which the photobioreactors are inventive and not conventional), the unit operations illustrated in FIG. 9 can be of conventional designs, or of straightforward adaptations or extensions of conventional designs, and can be selected and designed by those of ordinary skill in the chemical engineering arts using routine engineering and design principles.

In the illustrated, exemplary system, hot flue gases produced by electrical generating power plant facility 908 are, optionally, compressed in a compressor 910 and passed through a heat exchanger comprising a dryer 912, the function of which is explained below. Heat exchanger 912 is configured and controllable to allow the hot flue gas to be cooled to a desired temperature for injection into the photobioreactor arrays 902, 904, and 906. The gas, upon passing through the photobioreactors is treated by the algae or other photosynthetic organisms therein to remove one or more pollutants therefrom, for example, CO₂ and/or NO_{X}. Treated gas, containing a lower concentration of CO₂ and/or NO_{X} than the flue gas is released from gas outlets 914, 916, and 918 and, in one embodiment, vented to the atmosphere.

As described above, algae or other photosynthetic organisms contained within the photobioreactors can utilize the CO₂ of the flue gas stream for growth and reproduction thereby producing biomass. As described above, in order to maintain optimal levels of algae or other photosynthetic organisms within the photobioreactors, periodically biomass, for example in the form of wet algae, is removed from the photobioreactors through liquid medium outlet lines 921, 922, and 924.

From there, the wet algae is directed to dryer 912, which is fed with hot flue gas as described above. In the dryer, the hot flue gas can be utilized to vaporize at least a portion of the water component of the wet algae feed, thereby producing a dried algae biomass, which is removed via line 926. In certain embodiments, advantageously, dryer 912, in addition to drying the algae and cooling the flue gas stream prior to injection in the photobioreactors, also serves to humidify the flue gas stream, thereby reducing the level of particulates in the stream. Since particulates can potentially act as a pollutant to the photobioreactor and/or cause plugging of gas spargers within the photobioreactors, particulate removal prior to injection into the photobioreactors can be advantageous.

The water, or a portion thereof, removed from the wet algae stream fed to dryer 912 can be fed via line 928 to a condenser 930 to produce water that can be used for preparation of fresh photobioreactor liquid medium. In the illustrated embodiment, water recovered from condenser 930 (at “A”), after optional filtration to remove particulates accumulated in dryer 912, or other treatment to remove potential contaminants, can be pumped by a pump 932 to a medium storage tank 934, which feeds make up medium to the photobioreactors.

The dried algae biomass recovered from dryer 912 can be utilized directly as a solid fuel for use in a combustion device of facility 908 and/or could be converted into a fuel grade oil (e.g., “bio-diesel”) and/or a combustible organic fuel gas. In certain embodiments, as discussed below in the context of FIG. 10, at least a portion of the biomass can be utilized for the production of hydrogen therefrom. Algal biomass earmarked for oil production, fuel gas production, or hydrogen production (see FIG. 10) can be decomposed in a pyrolysis or other known gasification processes and/or a thermochemical liquefaction process to produce oil and/or combustible gas from the algae. Such methods of producing fuel grade oils and gases from algal biomass are well known in the art (e.g., see, Dote, Yutaka, “Recovery of liquid fuel from hydrocarbon rich microalgae by thermochemical liquefaction,” Fuel. 73:Number 12. (1994); Ben-Zion Ginzburg, “Liquid Fuel (Oil) From Halophilic Algae: A renewable Source of Non-Polluting Energy, Renewable Energy,” Vol. 3, No 2/3. pp. 249-252, (1993); Benemann, John R. and Oswald, William J., “Final report to the DOE: System and Economic Analysis of Microalgae Ponds for Conversion of CO₂ to Biomass.” DOE/PC/93204-T5, March 1996; and Sheehan et al., 1998; each incorporated by reference).

In certain embodiments, especially those involving combustion facilities for which it may be required by regulation to release the photobioreactor-treated gases into the atmosphere through a smoke stack of a particular height (i.e. instead of venting the treated gas directly to atmosphere as previously described), treated gas stream 936 could be injected into the bottom of a smoke stack 938 for release to the atmosphere. In certain embodiments, treated gas stream 936 may have a temperature that is not sufficient to enable it to be effectively released from a smoke stack 938. In such embodiments, cool treated-flue gas 936 may be passed through a heat exchanger 940 to increase its temperature to a suitable level before injection into the smoke stack. In one such embodiment, cooled treated flue gas stream 936 is heated in heat exchanger 940 via heat exchange with the hot flue gas released from the combustion facility, which is fed as a heat source to heat exchanger 940.

As is apparent from the above description, integrated photobioreactor gas treatment system 900 can provide a biotechnology-based air pollution control and renewable energy solution to fossil fuel burning facilities, such as power generating facilities. The photobioreactor systems can comprise emissions control devices and regeneration systems that can remove gases and other pollutants, such as particulates, deemed to be hazardous to people and the environment. Furthermore, the integrated photobioreactor system provides biomass that can be used as a source of renewable energy, (such as in the form of hydrogen, as discussed below and as illustrated in FIG. 10), thereby reducing the requirement of burning fossil fuels.

In addition, in certain embodiments, integrated photobioreactor combustion gas treatment system 900 can further include, as part of the integrated system, one or more additional gas treatment apparatus in fluid communication with the photobioreactors. For example, an effective, currently utilized technology for control of mercury and/or mercury-containing compounds in flue gases is the use of activated carbon or silica injection (e.g. see, “Mercury Study Report to Congress,” EPA-452/R-97-010, Vol. VIII, (1997); (hereinafter “EPA, 1997”), which is incorporated herein by reference). The performance of this technology, however, is highly temperature dependant. Currently, effective utilization of this technology requires substantial cooling of flue gases before the technology can be utilized. In conventional combustion facilities, this requires additional capital outlay and operational costs to install flue gas cooling devices.

Advantageously, because flue gases are already cooled within integrated system 900 through utilization of the flue gases for drying the algae in dryer 912, mercury and mercury-containing removal apparatus and treatments can readily and advantageously be integrated into the cool flue gas flow path, upstream 942 of the photobioreactors and/or downstream 944 of the photobioreactors. In either case, the reduced-temperature flue gas produced within integrated system 900 is highly compatible with known mercury controlled technologies, allowing a multi-pollutant (NO_{X}, CO₂, mercury) control system.

Similarly, a variety of known precipitation-based SO_{X} removal technologies also require cooling of flue gas (e.g. see, EPA, 1997). Accordingly, as with the mercury removal technologies discussed above, such SO_{X} precipitation and removal technologies could be installed in fluid communication with the photobioreactors in system 900 in similar locations (e.g., 942 and 944) as the above-described mercury removal systems.

As mentioned above, the present invention, in certain embodiments, also provides methods and systems of generating hydrogen with and/or from biomass comprising at least one species of photosynthetic organisms. In certain embodiments, the biomass is produced in a photobioreactor; in such embodiments, or other embodiments, the biomass is algal biomass comprising algae. In certain such embodiments, because biomass containing a high percentage of starch-like materials may be well suited for generating hydrogen therefrom, the algal biomass comprises species of microalgae that are starch-accumulating. A variety of such starch-accumulating species of algae are known to those skilled in the art. In certain other embodiments, biomass utilized, according to the invention, for generating hydrogen need not, necessarily, comprise the algal materials mentioned immediately above, but, rather, may, in part or in whole, be derived from essentially any suitable source and/or may be produced in any suitable photobioreactor, including conventional photobioreactors, fed with any of a wide variety of fuel sources for photosynthesis, for example atmospheric air, flue gas, purified CO₂, etc. In certain embodiments, the inventive hydrogen production systems and methods utilize photobioreactors that are similar to or identical in design, configuration, and/or operation to those previously described in FIGS. 1, 2, and 6a or in FIGS. 3 and 3a. Moreover, in certain embodiments, such as certain embodiments of hydrogen generation processes 1000 illustrated in FIG. 10 and described below, a hydrogen production system, according to the invention, is part of an integrated combustion and hydrogen production method and system employing photobioreactors, which produce the biomass used for hydrogen generation, that are configured to mitigate pollutants from combustion gases, as previously described in the context of system 900 of FIG. 9.

In certain such embodiments, the photobioreactors forming part of the inventive hydrogen production system are utilized as part of an overall combustion system, wherein they are fed combustion gases comprising pollutants such as CO₂ and/or NOₓ. In such embodiments, the hydrogen generating systems and methods, such as described below in the context of FIG. 10, are utilized as part of an overall hydrogen production system in which one or more photobioreactors producing biomass utilized for hydrogen generation are also utilized for mitigating greenhouse, especially CO₂, from the emissions of combustion facilities, such as power plants, incinerators, etc. and converting at least a portion of the greenhouse gases mitigated into a substrate (biomass) for the production of hydrogen. As explained in detail below, in such embodiments, the present invention enables production of hydrogen as part of an overall system that also serves to reduce CO₂ and NOₓ emissions from, and fossil fuel use by, power plants and other combustion facilities.

The inventive methods and systems for generating hydrogen with and/or from biomass using a hydrogen production system and method in which photobioreactors producing the biomass are also used for converting CO₂ emissions from combustion facilities into the same biomass used for generating hydrogen provide an advantageous way of producing hydrogen from a renewable energy source (i.e. solar energy) that is environmentally friendly and economically attractive. Such an integrated combustion gas mitigation/hydrogen production system is environmentally friendly because, as explained in more detail below, such a system can involve net-zero CO₂ emissions and/or NOₓ mitigation. For example, in certain embodiments, CO₂ released in by-product gas produced during the generation of hydrogen from biomass is balanced or exceeded by the level of CO₂ mitigation in combustion gases when the biomass utilized for hydrogen generation is produced, by photosynthesis, in the photobioreactors. In short, any CO₂ released during the hydrogen generation is more than compensated for by the amount of CO₂ removed from combustion gas by the photobioreactors of the integrated hydrogen production system.

Moreover, in certain embodiments, as discussed in more detail below in the context of FIG. 10, CO₂ by-product, or a portion thereof, produced during the generation of hydrogen from the biomass can be recycled to the inlet(s) of the photobioreactor(s) of such an integrated system and “recycled” to produce additional biomass. Such a closed-loop CO₂ recycle can, in certain embodiments, even further reduce, and in certain embodiments almost entirely eliminate, overall CO₂ emissions from the hydrogen generation system. Similarly, NOₓ and/or other by-products produced during generation of hydrogen from biomass can be recycled to the inlet(s) of the photobioreactor(s) for mitigation. In addition, since biomass, such as algal biomass, creation is solar-driven, a major feed stock and energy source (the sun) utilized for hydrogen generation is renewable—at least for the foreseeable future! This is in stark contrast to typical conventional hydrogen generation systems that rely solely on natural gas and/or other fossil fuels (e.g. coal) as feed stocks for hydrogen generation.

A variety of exemplary hydrogen generating systems and methods, according to the invention, are illustrated in the schematic flow diagrams of FIG. 10. As mentioned above, and as discussed in further detail below, in certain embodiments, the hydrogen generation systems and methods 1000 illustrated in FIG. 10 may form part of an overall hydrogen production system including one or more photobioreactors that are utilized to produce the biomass 1002 used as feed stock for producing hydrogen gas 1004. In the discussion below, the hydrogen generation systems and methods illustrated in FIG. 10 are discussed in the context of an example in which hydrogen generation systems 1000 form part of an overall integrated combustion and hydrogen production method and system, in which the hydrogen generation system (e.g. system 1006 or 1007) is integrated with a combustion gas pollution mediation system, for example as described above and illustrated in FIG. 9. However, it must be noted that while the discussion below refers to the hydrogen generation systems and methods 1000 in the context of an overall combustion and hydrogen production system and method, the inventive hydrogen generation systems and methods are not so limited and can, in other embodiments, utilize biomass produced from other sources and by other methods and systems.

In addition, according to certain embodiments, the invention can involve a method for facilitating or promoting the production of hydrogen comprising providing biomass that is produced in a photobioreactor for the purpose of generating hydrogen therefrom. Such biomass produced in a photobioreactor may, in certain embodiments, have been produced by any of the systems and methods described previously, but, in other embodiments, need not have been so produced. In certain such embodiments, the biomass is produced in a photobioreactor during mitigation of pollutants such as CO₂ and/or NOₓ from combustion gases or other gas emissions. In certain such embodiments, optionally, such an inventive method also can involve producing the biomass provided for hydrogen generation. As used herein “facilitating” or “promoting” includes all methods of doing business including methods of education, industrial and other professional instruction, energy industry activity including sales of biomass, and any advertising or other promotional activity including written, oral and electronic communication of any form, associated with biomass produced in a photobioreactor in connection with using such biomass for the generation of hydrogen from such biomass. In certain embodiments, such inventive methods of promoting or facilitating the production of hydrogen can further comprise providing instructions for generating and/or directions as to how to generate hydrogen from such biomass. “Instructions” or “directions” can and often do define a component of promotion or facilitation, and typically involve written instructions. Instructions and directions can also include any oral and/or electronic instructions provided in any manner.

In yet another embodiment, the invention involves producing hydrogen from biomass produced in a photobioreactor. Such a method could, for example, involve obtaining biomass that was produced in a photobioreactor from a third party and generating hydrogen from the biomass. Such biomass produced in a photobioreactor may, in certain embodiments, have been produced by any of the systems and methods described previously, but, in other embodiments, need not have been so produced. In certain such embodiments, the biomass is produced in a photobioreactor during mitigation of pollutants such as CO₂ and/or NOₓ from combustion gases or other gas emissions.

Referring to FIG. 10, hydrogen generating systems 1000 are now discussed in more detail in the context of an example wherein system 1006 and/or 1007 is integrated within an overall hydrogen production and combustion gas treatment system in which the system components and process steps illustrated in FIG. 10 are integrated with the systems and methods illustrated and discussed above in the context of FIG. 9. As above for system 900 of FIG. 9, certain embodiments of hydrogen generating systems 1000 can utilize unit operations and sub-systems that are either of conventional design or are straight-forward adaptations or extensions of conventional designs, which can be selected and designed by those of ordinary skill in the chemical engineering art and/or other engineering arts using routine engineering and design principles or straight-forward adaptations or extensions thereof.

The process flow diagram illustrated in FIG. 10 shows two alternative systems and methodologies for generating hydrogen gas 1004 from the exemplary feed stock of algal biomass 1002: (1) system 1006 comprises a combination of pyrolysis/gasification system 1008 and steam reforming/water gas-shift (WGS) reaction system 1010; and system 1007, which utilizes bacterial fermentation system 1026. It should be noted that in alternative embodiments, technologies and systems for producing hydrogen gas from biomass other than those illustrated in FIG. 10, including those not yet developed or conceived of as of the filing date of the present application, can potentially be used to generate hydrogen gas according to the invention. Accordingly, the systems and methods described herein in the context of FIG. 10 are meant to be exemplary and not limiting.

In one alternative embodiment, for example, photosynthetic organisms, such as algae, are used to produce hydrogen directly as a by product of their metabolism. In such embodiments, hydrogen could be produced directly by algal cultures that are present in a photobioreactor, such as one or more of photobioreactors 902, 904, or 906, without the need for harvesting the algal biomass from the photobioreactor and converting it to hydrogen (i.e. producing hydrogen from the biomass), as described in the context of FIG. 10. In certain embodiments, hydrogen may be produced both directly by algae as a by-product of their metabolism and by converting harvested algal biomass to hydrogen, as described in the context of FIG. 10. In an exemplary embodiment of hydrogen production via growth of algal cultures themselves, an appropriate algae, such as Dunaliella salina, is used produce hydrogen gas. In such an embodiment, the algae is induced, either continuously or intermittently, to cease emitting oxygen and stop storing energy as carbohydrates, protein and fats by imposing a nutrient stress (e.g. a sulfur deficiency) within the system (e.g. via use of a sulfur-deficient medium). Instead, the algal cells are induced to use an alternative metabolic pathway to exploit stored energy reserves, anaerobically, in the absence of oxygen. As hydrogenase (a key enzyme in hydrogen production) becomes activated in such a pathway, large amounts of hydrogen gas from water can be formed and released as a byproduct.

Returning to FIG. 10, in certain embodiments, biomass 1002 can be utilized to generate hydrogen gas 1004 via a hydrogen generating system, such as system 1006, comprising one or more systems for performing pyrolysis/gasification (1008) in combination with one or more systems 1010 configured to react gas produced by pyrolysis/gasification system 1008 with a reactant, typically water, to produce a product stream comprising hydrogen. In certain embodiments, optionally, the product gas from system 1010 can be treated with one or more gas separation devices, such as system 1012, to separate hydrogen gas 1004 from other by-product gases.

It should be noted that while in the illustrated embodiment hydrogen generating system 1006 illustrates pyrolysis/gasification as occurring in one system or set of unit operations 1008, steam reforming/WGS reaction as occurring in another system/series of unit operations 1010, and gas separation occurring in yet another system/series of unit operations 1012, in other embodiments, these steps/systems may be combined and/or performed in fewer or more steps/systems/series of unit operations than illustrated or in a single step/system/series of unit operations. For example, certain known gasification technologies can involve reactors wherein gasification and catalytic steam reforming can occur in a single, combined-function reaction vessel. Such alternative systems and technologies for generating synfuel and/or hydrogen are within the scope of the present invention.

Referring to system 1006, feed stock 1002 comprising algal biomass is first subjected to pyrolysis or gasification 1008 to produce an organic biogas/syngas 1014, which is subsequently reacted with water in system 1010 to produce a product gas 1022 comprising hydrogen 1004.

Biomass feed stock 1002, in certain embodiments, can comprise wet algae, such as that fed to dryer 912 of system 900, or dry algal biomass, such as that removed via line 926 from system 900. Pyrolysis/gasification, as performed in system and step 1008, can comprise any of a wide variety of suitable known pyrolysis/gasification systems and methods, such as those described and referred to previously in the context of system 900.

In certain embodiments, system 1008 comprises a gasification system producing organic gases, herein referred to as biogas or, equivalently, syngas. Typical conventional gasification systems involve a combination of pyrolysis of organic feedstock and a secondary step of gasification of char and ash produced as a by-product of the pyrolysis reactions to form additional syngas. In certain embodiments the steps of pyrolysis and char conversion take place in separate unit operations. However, in certain embodiments, these steps and reactions are combined and take place in a single reaction vessel.

Typically, gasification is a two-step, endothermic process in which a solid or liquid organic fuel is thermally, chemically converted into a low-or medium-Btu organic syngas. In a first reaction, pyrolysis, volatile components of the organic fuel are converted into organic syngas vapor at elevated temperature, but typically below 600° C., by a set of complex chemical reactions. Typically included in the syngas thereby produced are hydrocarbon gases, hydrogen, CO, CO₂, and water vapor. In addition, NOₓ can be produced as a by-product. Char (fixed carbon) and ash comprise pyrolysis by-products, which are not vaporized in the pyrolysis step. In a second step and series of reactions, such char is gasified through reactions with oxygen, steam, and hydrogen. In certain embodiments, such char conversion reactions need not be performed by the system and the system can be configured to perform only pyrolysis.

A variety of known gasification and gasifier technologies for gasifying organic feedstocks (e.g. coal, crop wastes, waste plastics, etc.) are available or have been proposed that are suitable, potentially suitable, or could be modified for use with biomass in the context of the present invention. Such gasifiers can be either of a fixed-bed design or fluidized-bed design. Gasifiers can be either air-blown or fed with a gas stream comprising pure oxygen or oxygen-enriched air. Gasifiers employing oxygen or oxygen-enriched air typically generate a syngas having a higher Btu value and provide faster reaction rates than air-blown systems. In the illustrated embodiment, gasification system 1008 could advantageously utilize the oxygen-enriched gas streams released from photobioreactors 902, 904, and 906 of system 900 to improve performance of the system, as shown by dashed line 1016. Steam and energy/heat input requirements of the pyrolysis/gasification system 1008 can also be reduced, in certain embodiments, by utilizing hot flue gas 1018, obtained from, for example, electrical generating power plant facility 908, to provide heat energy to help drive the endothermic pyrolysis reactions, and steam 1020, produced by algae dryer 912 of system 900, can be fed to pyrolysis/gasification system 1008 for utilization therein. In addition, any cooled flue gas that was used for supplying heat energy to system 1008, as well as any waste/by-product gases produced by the system, which can contain CO₂ and NOₓ may be, in certain embodiments, recycled to system 900 and fed to photobioreactors 902, 904, and 906 for mitigation of CO₂ and other pollutants, such as NOₓ.

Conversion of organic biogas 1014 to hydrogen in system 1006 can occur via one or both of steam reforming or WGS reaction in sub-system 1010. Typical conventional steam reforming technologies and systems utilize a catalytic process that involves a reaction between organic gases in syngas, such as, for example, methane and other light hydrocarbons, with steam. The reforming step catalytically reacts these organic gases with steam in a exothermic reaction to form hydrogen and CO. In a second reaction, the water gas-shift (WGS) reaction, the CO is then “shifted” with steam (typically at 700-1100° C.) to form additional hydrogen and CO₂ in an endothermic reaction. A wide variety of systems and processes for performing these reactions are known and available to those skilled in the art.

Thus, in certain embodiments of reforming/WGS reaction system 1010 of system 1006, biogas 1014 is reacted in a two step process to produce a product gas 1022 including hydrogen with CO₂ as a major by-product. As previously discussed in the context of pyrolysis/gasification subsystem 1008, to reduce the overall energy and steam requirements of system 1010, hot flue gas from combustion device 908 of system 900 may be utilized as a heat source and at least a portion of the steam required for the reactions may be derived from steam produced by dryer 912 of system 900 during the drying of algae harvested from the photobioreactors in producing dry algal biomass.

Product gas 1022 exiting subsystem 1010, which is enriched in hydrogen and CO₂ can, optionally, undergo gas separation by optional gas separation system 1012 to separate purified product hydrogen gas 1004 from by-product gases 1024. A wide variety of well known, mature gas separation technologies can be utilized for performing such gas separation in system 1012 including, but not limited to, membrane-based separation processes and/or pressure swing adsorption (PSA) gas separation technologies. Such gas separation technologies and systems are well known and readily available to those skilled in the art. By-product gas stream 1024 can, in certain embodiments, be vented to the atmosphere or, advantageously, because it is enriched in CO₂ and may contain other pollutant by-products, such as NOₓ, be recycled as a feed to the bioreactors of flue gas mitigation system 900 shown in FIG. 9. Because separation of NOₓ from hydrogen product can be an expensive process, for certain embodiments where NOₓ is produced by the hydrogen generation methods utilized (e.g. pyrolysis), and where it is desirable to obtain a hydrogen product from which NOₓ has been at least partially removed and/or where it is desirable to recycle by-product NOₓ to photobioreactors for mitigation and/or where it is desirable to minimize atmospheric release of NOₓ during hydrogen generation, it can be desirable to reduce or minimize the nitrogen content of the biomass used for hydrogen generation.

A description of certain pyrolysis and gasification systems and technology and of certain technology, processes, and systems for catalytic steam reforming and WGS reaction formation of hydrogen can be found in a wide variety of texts, articles, and other references including, but not limited to, the following: Evans R., et al., “Hydrogen from Biomass: Catalytic Reforming of Pyrolysis Vapors” US DOE Hydrogen, Fuel Cells & Infrastructure Technologies Program-2003 Annual Merit Review Meeting, May 18-22, 2003, Berkeley Calif.; Magrini-Bair K., et al. “Fluidizable Catalysts for Hydrogen Production from Biomass Pyrolysis/Steam Reforming”, US DOE Hydrogen, Fuel Cells & Infrastructure Technologies Program-2003 Annual Merit Review Meeting, May 18-22, 2003, Berkeley Calif.; Czernik S., et al. “Hydrogen from Post-Consumer Residues”, US DOE Hydrogen, Fuel Cells & Infrastructure Technologies Program-2003 Annual Merit Review Meeting, May 18-22, 2003, Berkeley Calif.; Chomet E. and Czernik S. “Renewable Fuels: Harnessing Hydrogen”, Nature, vol. 418, pp. 928-929(2002); Czemik S., et al. “Hydrogen by Catalytic Steam Reforming of Liquid Byproducts from Biomass Thermoconversion Processes”, I&EC Research, vol. 41, pp. 4209-4215 (2002), Antal, Jr., M. J., S. G. Allen, D. Schulman, and X. Xu. 2000. Biomass Gasification in Supercritical Water. Industrial Engineering Chemical Research, Vol. 39, pp. 4040-4053; Antal, Jr., M. J., G. Varhegyi, and E. Jakab. (1998) “Cellulose Pyrolysis Kinetics: Revisited:, Industrial Engineering Chemical Research, Vol. 37, pp. 1267-1275; and Cortrigh, R. D., R. R. Davda and J. A. Dumesic. (2002) “Hydrogen from Catalytic Reforming of Biomass-derived Hydrocarbons in Liquid Water”, Nature Vol. 418, 964-967; all of which are incorporated herein by reference

In an alternative embodiment, instead of utilizing catalytic steam reforming technology and unit operations in system 1010, system 1010 can comprise a biological WGS fermenter/reactor that utilizes strains of photosynthetic bacteria isolated from the natural environment that are able to perform the WGS reaction at ambient temperatures. Such a biological WGS system and method for converting syngas to hydrogen is described in: Wolfrum E., et al. “Biological Water Gas Shift”, US DOE Hydrogen, Fuel Cells & Infrastructure Technologies Program-2003 Annual Merit Review Meeting, May 18-22, 2003, Berkeley Calif.; Maness and Weaver “Hydrogen Production From a Carbon-Monoxide Oxidation Pathway in Rubrivivax gelatinosus”, International J Hydrogen Energy, vol. 27, pp. 1407-1411 (2002), each incorporated herein by reference).

In addition, in alternative embodiments, algal biomass 1002 can be converted to hydrogen gas directly by utilizing the biomass as a substrate for bacterial fermentation/digestion as illustrated in system and process 1007. Such a system can utilize a bacterial fermentation system 1026 configured to perform a fermentation process, such as an anaerobic fermentation process, to convert organic substrates, such as algal biomass 1002 and/or organic products produced from a fermentation of such biomass, to hydrogen-rich gas 1028, which can, optionally, be separated from by-product gases by gas separation system 1012, as described previously. Potentially advantageously, after bacterial digestion of the biomass to produce hydrogen, the residual bacterial slurry may, in certain embodiments, contain the nitrogen component of the biomass used as feed, thereby eliminating or reducing production of NOₓ by-product by the process. Such bacterial fermentation systems for producing hydrogen directly or via sequential fermentations from organic materials and organic wastes, which are potentially suitable for use in the context of the present invention, are known in the art and have been described in: Sung S., et al. “Biohydrogen Production from Renewable Organic Wastes”, US DOE Hydrogen, Fuel Cells & Infrastructure Technologies Program-2003 Annual Merit Review Meeting, May 18-22, 2003, Berkeley Calif.; Van Ginkel, S, et al. “Biohydrogen Production as a Function of pH and Substrate Concentration”, Environmental Science & Technology, vol. 35, pp. 4726-4730 (2001); Ike, A., et al. “Algal CO₂ fixation and H₂ Photoproduction,” In BioHydrogen, Zaborsky et al., eds. Plenum Press, New York, pp. 265-271 (1998); Ikuata, Y., et al. “Hydrogen Production By Photosynthetic Microorganisms,” In BioHydrogen, Zaborsky et al., eds. Plenum Press, New York, pp. 319-327 (1998); Miura, Y., et al. “Stimulation of Hydrogen Production in Algal Cells Grown Under High CO₂ Concentration and Low Temperature,” Applied Biochemistry and Biotechnology, vol. 39/40: pp. 753-761 (1993); and Ike, A., et al. “Hydrogen Photoproduction from CO₂-Fixing Microalgal Biomass: Application of Lactic Acid Fermentation by Lactobacillus amylovorus,” Journal of Fermentation and Bioengineering, vol. 84, pp. 428-43³ (1997); each of which is incorporated herein by reference.

As is apparent from the above description, hydrogen generating systems 1000 as illustrated in FIG. 10, especially when integrated with a photobioreactor gas treatment system such as system 900 of FIG. 9, can provide a biotechnology-based hydrogen production system that can provide both clean hydrogen fuel as well as mitigation of pollutants and greenhouse gases while, simultaneously, reducing the amount of fossil fuel necessary to produce both energy and hydrogen over currently available technologies. Moreover, because the hydrogen fuel produced by the systems and methodologies 1000 of FIG. 10 utilize biomass such as algae, as opposed to fossil fuels, as a feed source, certain embodiments of the inventive hydrogen generating systems and methods can provide hydrogen fuel without exacerbating the depletion of fossil fuel reserves and without generating additional CO₂ emissions.

The function and advantage of these and other embodiments of the present invention may be more fully understood from the examples below. The following examples, while illustrative of certain embodiments of the invention, do not exemplify the full scope of the invention.

### EXAMPLE 1

### Mitigation of CO₂ and NOₓ with a Three-Photobioreactor Module Including Three Triangular Tubular Photobioreactors

Each photobioreactor unit of the module utilized for the present example comprised 3 tubes of essentially circular cross-section constructed from clear polycarbonate, assembled as shown in FIG. 1, with α₁=about 45 degrees and α₂=about 90 degrees. In this essentially triangular configuration, the essentially vertical leg was 2.2 m high and 5 cm in diameter; the essentially horizontal leg was 1.5 m long and 5 cm in diameter; and the hypotenuse was 2.6 m long and 10 cm in diameter. The photobioreactor module comprised 3 adjusted units arranged in parallel, similarly as illustrated in FIG. 2. This bioreactor module has a footprint of 0.45 m²

A gas mixture (certified, AGA gas), mimicking flue gas composition was used (Hiroyasu et al., 1998). The total gas flow input was 715 ml/min per each 10 liter photobioreactor in the module. Gas distribution to the spargers injecting gas into the vertical legs and the to the spargers injecting gas into the hypotenuse legs was 50:50. Mean bubble size was 0.3 mm. CO₂ and NOₓ composition at the bioreactor inlet and outlet ports was measured using a flue gas analyzer (QUINTOX™; Keison Products, Grants Pass, Oreg.).

Light source, applied only to the hypotenuse legs, was a full-spectrum “SUNSHINE™” lamps, with a radiation intensity of 390 W/m². Light radiation was measured with using TES light meter (TES Electrical Electronic Corp., Taipei, Taiwan, R.O.C.). Light cycle was 12 h light-12 h dark. The temperature was maintained at 26 degrees C.

Algal heat value was measured using a micro oxygen bomb calorimeter per Burlew, 1961.

The microalgae Dunaliella parva (UTEX.) culture was used as a model. It was specifically chosen for its proven track record in large scale production, tolerance to flue gas composition and, ability to produce high-quality biofuel.

Medium used was modified F/2 containing:

22 g/l NaCl, 16 g/l Artificial Sea Water Sea Salts (INSTANT OCEAN®, Aquarium Systems, Inc. Mentor, Ohio), 0.425 g/l NaNO₃, 5 g/l MgCl₂, 4 g/l Na₂SO₄, and 1 ml Metal Solution per liter medium (see contents of stock solution below)+5 ml Vitamin Solution (see contents of stock solution below) per liter medium. The pH was maintained at pH 8.

Stock Solution Compositions:

Metal-Solution—Trace metals stock solution (chelated) per liter

| | |
|---|---|
| EDTANa₂ | 4.160 g |
| FeCl₃.6H₂O | 3.150 g |
| CuSO₄.5 H₂O | 0.010 g |
| ZnSO₄.7 H₂O | 0.022 g |
| CoCl₂.6 H₂O | 0.010 g |
| MnCl₂.4 H₂O | 0.180 g |
| Na₂MoO₄.2 H₂O | 0.006 g |

Vitamin Solution— Vitamin stock solution per liter

| | |
|---|---|
| Cyanocobalamin | 0.0005 g |
| Thiamine HCl | 0.1 g |
| Biotin | 0.0005 g |

Cell density was calculated using spectrophotometer measurements at 680 nm (see, Hiroyasu et al., 1998).

Under the experimental conditions, the following performance was achieved:
90% CO₂ mitigation (in the presence of light);
98% and 71% NOₓ removal (in light and dark, respectively);
solar efficiency of 19.6%.

### EXAMPLES 2-5

### Photobioreactor Arrays for Mitigation of Power Plant Flue gas Pollutants and Production of Algal Biomass

All examples below relate to a 250 MW, coal-fired power plant with a flue gas flow rate of 781,250 SCFM, and coal consumption of 5,556 tons/d. Flue gas contains CO₂ (14% vol), NOx (250 ppm) and post-scrubbing level of SOx (200 ppm, defined in the US 1990 Clean Air Act Amendment). 12 h/d sunlight is assumed, as is a mean value of solar radiation of 6.5 kWh/m²/d, representing typical South-Western US levels (US Department of Energy). Algal solar efficiency of 20% is assumed, based on performance data of Example 1 and literature values (Burlew, 1961). Daytime algal CO₂ and NOₓ mitigation efficiency is 90% and 98% (respectively), and at night 0% and 75% (respectively), based on Example 1 performance and literature values (Sheehan et al., 1998; Hiroyasu et al., 1998). Biodiesel production potential is 3.6 bbl per ton of algae (dry weight) (Sheehan et al., 1998). System size and performance for various capacities and operating protocols are summarized below in Table 2.

TABLE 2
Examples 2-5 Size and Capacity Estimates

| | | % of total | Bioreactor | | | | | Algal | | Renewable |
|---|---|---|---|---|---|---|---|---|---|---|
| | | flue gas | operation | Overall | CO₂ | Overall | NOₓ | biomass | Biodiesel | power |
| | Footprint | produced | mode | % CO₂ | mitigated | % NOₓ | removed | production | production | production*** |
| Example | (km²) | processed | (h/day) | mitigated* | (tons/y) | mitigated** | (tons/y) | tons(dw)/y | (bbl/y) | MW |
| 2 | 0.45 | 11 | 12 | 5 | 81,000 | 6 | 170 | 31,000 | 111,600 | 7 |
| 3 | 0.45 | 11 | 24 | 5 | 81,000 | 9 | 290 | 31,000 | 111,600 | 7 |
| 4 | 0.45 | 100 | 24 | 5 | 81,000 | 85 | 2,600 | 31,000 | 111,600 | 7 |
| 5 | 1.3 | 33 | 12 | 15 | 244,000 | 17 | 520 | 95,000 | 342,000 | 22 |

*CO2 avoided basis
**NOₓ avoided basis
***Assuming 35% power plant efficiency

### EXAMPLE 6

### Use of a Small-Scale Automated Photobioreactor Cell Culture System for Preconditioning of Algal Cultures to High Intensity Illumination and Photomodulation

A culture of the microalgae Dunaliella parva (UTEX.) was grown and adapted, as described below, using a small-scale photobioreactor system similar to that illustrated in FIGS. 8a-8f. The medium used was the same modified F/2 described in Example 1. The cell culture module had an internal culture volume of about 10 ml. Gas exchange was performed utilizing a silicone-coil gas exchanger, similar to gas exchanger 862 of FIG. 8a, which was fed a gas mixture comprising 8% CO₂ (balance air) at a rate of 100 ml/min. Flow rate of liquid medium in the perfusion loop was about 1 mmin net forward flow. The culture was stirred using magnetic stir bars rotated at about 40 RPM. The culture was maintained at room temperature (about 25° C.). Cell density was monitored with a spectrophotometer, and culture dilutions were made as necessary to maintain growth of the culture (maintained within an operating range near the upper end of the concentration in which the algae is still in the log growth regime). Typically, such dilutions were performed at least once per day during the adaptation period. Initially, the culture was grown under steady illumination of about 150 μEm⁻²s⁻¹. The above conditions are referred to below as the “initial conditions.”

In a test culture, illumination intensity was increased by 50 μm⁻²s⁻¹ once per day until a level of 300 μm⁻²s⁻¹ was reached. At this point, a light source modulator utilizing a chopper wheel (similar to light source modulator 830 illustrated in FIGS. 8a and 8g) was used to subject the test culture to a photomodulation pattern of repetitive cycles of 0.5 second light exposure followed by 0.2 second dark exposure. This photomodulation pattern was maintained for the rest of the adaptation period for the test culture. For the remainder of the adaptation period, light intensity was increased once per day in 50 μEm⁻²s⁻¹ intervals until an illumination intensity of 2,000 μEm⁻²s⁻¹ was reached. Total adaptation time was about 40 days, with the final conditions referred to below as the “test conditions.”

At the end of this period, a control culture grown only under the initial conditions was exposed to culture at the test conditions and growth rate was measured for both the adapted culture and the control culture under the test conditions. It was found that the doubling time of the control culture grown under the test conditions was about 20 hours, while that of the adapted culture was about 6 hours.

### EXAMPLE 7

### Photobioreactor Arrays for Mitigation of Power Plant Flue gas Pollutants and Production of Hydrogen from Algal Biomass

This example is based on a 250 MW coal-fired power plant and photobioreactor array system similar to that described above for Examples 2-5, except that the power plant produces flue gas at an average flow rate of 781,250 SCFM, the total flue gas (100%) is processed, the overall % CO₂ mitigated is 1% (16,200 tons/y), the overall % NO_{X} mitigated is 85% (2,600 ton/y), and the footprint of the photobioreactor array system is 0.13 km². The photobioreactor array produces 6,200 tons(dw)/yr of algal biomass. This biomass (6,200 tons/yr) is converted to 2,000 tons/y of hydrogen using a hydrogen generating system that comprises biomass gasification, catalytic steam reforming, and hydrogen gas separation and purification systems, such as hydrogen generation system 1006 illustrated in FIG. 10 and described above.

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood, unless otherwise indicated, to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Any terms as used herein related to shape, orientation, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elipitical/elipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described.

In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

1. A method of producing hydrogen comprising acts of:
growing at least one species of algae in an enclosed photobioreactor system exposed to sunlight as a source of light driving photosynthesis; and
generating hydrogen with the algae.

2. A method as in claim 1, wherein the enclosed photobioreactor system comprises a plurality of enclosed photobioreactor apparatuses.

3. A method as in claim 1, further comprising after the growing act, an act of:
harvesting at least a portion of the photosynthetic organisms from the bioreactor to form biomass.

4. A method as in claim 3, wherein the generating act comprises generating hydrogen from the biomass.

5. A method as in claim 1, wherein the generating act comprises generating hydrogen via metabolism of the at least one species of algae in the enclosed photobioreactor system.

6. A method of producing hydrogen comprising:
growing at least one species of algae in a photobioreactor system comprising a plurality of enclosed photobioreactor apparatuses; and
generating hydrogen with the algae.

7. A method as in claim 6, further comprising after the growing act, an act of:
harvesting at least a portion of the photosynthetic organisms from the bioreactor to form biomass.

8. A method as in claim 7, wherein the generating act comprises generating hydrogen from the biomass.

9. A method as in claim 6, wherein the generating act comprises generating hydrogen via metabolism of the at least one species of algae in the photobioreactor system.

10. A method as in claim 6, wherein at least one of the photobioreactor apparatuses comprises at least a first, a second, and a third fluidically interconnected conduits, at least one of which is at least partially transparent to light of a wavelength capable of driving photosynthesis, the conduits together providing a flow loop enabling a liquid medium contained within the photobioreactor to flow sequentially from a region of origin within the flow loop through the first, second, and third conduits and back to the region of origin.

11. A method as in claim 10, wherein the region of origin comprises a liquid header in fluid communication with one end of the first conduit and one end of the third conduit.

12. A method as in claim 11, wherein the photobioreactor system further comprises a second liquid header in fluid communication with one end of the second conduit and the other end of the third conduit.

13. A method as in claim 12, wherein the first liquid header and the second liquid header are elongated end are in fluid communication with a plurality of fluidically interconnected conduits that are arranged to provide a plurality of flow loops, each of the flow loops comprising one of the plurality of enclosed photobioreactor apparatuses, and each of the flow loops comprising at least a first, a second, and a third fluidically interconnected conduit fluidically interconnected to each other such that the liquid medium contained within each flow loop is able to flow sequentially from the first header through the first conduit to and through the second conduit, into the second header, and through the third conduit so that the liquid returns to the first liquid header.

14. A method of producing hydrogen comprising acts of:
providing a liquid medium comprising at least one species of photosynthetic organisms within an enclosed photobioreactor;
exposing at least a portion of the photobioreactor and the at least one species of photosynthetic organisms to sunlight as a source of light driving photosynthesis;
harvesting at least a portion of the photosynthetic organisms from the bioreactor to form biomass; and
generating hydrogen from the biomass.

15. A method as in claim 14, further comprising drying the biomass.

16. A method as in claim 14, wherein the generating act further comprises an act of gasification or pyrolysis of the biomass.

17. A method as in claim 16, wherein the generating act further comprises reacting a gas produced by the gasification or pyrolysis act to form hydrogen gas.

18. A method as in claim 14, wherein the generating act comprises subjecting the biomass to bacterial digestion.

19. A method as in claim 14, comprising establishing a flow of the liquid medium comprising at least one species of photosynthetic organisms within the photobioreactor.

20. A method as in claim 19, further comprising acts of:
calculating a first exposure interval of the photosynthetic organisms to the light at an intensity sufficient to drive photosynthesis and a second exposure interval of the photosynthetic organisms to dark or the light at an intensity insufficient to drive photosynthesis required to yield a selected growth rate of the photosynthetic organisms within the photobioreactor; and
controlling the flow of the liquid medium within the photobioreactor based on the exposure intervals determined in the calculating act.

21. A method as in claim 19, further comprising acts of:
performing a simulation of liquid flow patterns within the photobioreactor and, from the simulation, determining a first exposure interval of the photosynthetic organisms to light at an intensity sufficient to drive photosynthesis and a second exposure interval of the photosynthetic organisms to dark or light at an intensity insufficient to drive photosynthesis;
calculating from the first exposure interval and the second exposure interval a predicted growth rate of the photosynthetic organisms within the photobioreactor; and
controlling the flow of the liquid medium within the photobioreactor so as to yield a selected first exposure interval and a selected second exposure interval of the photosynthetic organisms to achieve a desired predicted growth rate as determined in the calculating act.

22. A method as in claim 14, further comprising acts of:
introducing a stream of gas to be treated to the photobioreactor; and
at least partially removing from the gas with the photobioreactor CO₂ and/or NOₓ.

23. A method as in claim 22, wherein the gas introduced in the introducing act comprises combustion gas derived from a power generating apparatus and/or an incinerator.

24. A method as in claim 20, wherein in the controlling act, the flow of the liquid medium is controlled utilizing a computer implemented system configured to perform a simulation of liquid flow patterns within the photobioreactor, and, from the simulation, to determine a computed actual first exposure interval of the photosynthetic organisms to the light at an intensity sufficient to drive photosynthesis and a second computed actual exposure interval of the photosynthetic organisms to dark or the light at an intensity insufficient to drive photosynthesis and to establish a flow of the liquid medium within the bioreactor selected to minimize the difference between the computed actual first and second exposure intervals and the first and second exposure intervals calculated in the calculating act.

25. A method as in claim 21, wherein predicted growth rate calculated in the calculating act from the first and second exposure intervals is determined utilizing a mathematical model that simulates the growth rate of the photosynthetic organisms when exposed to alternating periods of exposure to light at an intensity sufficient to drive photosynthesis and exposure to light at an intensity insufficient to drive photosynthesis.

26. A method as in claim 25, further comprising, before the calculating act:
determining at least one adjustable parameter of at least one equation utilized in the mathematical model by curve fitting the at least one equation to growth rate versus light exposure interval data generated using a pilot-scale bioreactor containing a liquid medium comprising the at least one species of photosynthetic organisms.

27. A method as in claim 24, wherein the first and second exposure intervals required to yield a selected growth rate calculated in the calculating act are determined utilizing a mathematical model that simulates the growth rate of the photosynthetic organisms when exposed to alternating periods of exposure to light at an intensity sufficient to drive photosynthesis and exposure to light at an intensity insufficient to drive photosynthesis.

28. A method as in claim 27, further comprising, before the calculating act:
determining at least one adjustable parameter of at least one equation utilized in the mathematical model by curve fitting the at least one equation to growth rate versus light exposure interval data generated using a pilot-scale bioreactor containing a liquid medium comprising the at least one species of photosynthetic organisms.

29. A method as in claim 24, wherein the photobioreactor comprises at least a first and a second fluidically interconnected conduits, a first gas sparger configured and positioned to introduce a gas stream into the first conduit, and a second gas sparger configured and positioned to introduce a gas stream into the second conduit, and wherein
the computer implemented system is further configured to control the flow of the liquid medium within the photobioreactor by controlling the overall flow rate of the gas to be treated by the photobioreactor and the distribution of the overall flow rate of the gas to the first and second gas spargers.

30. A method as in claim 19, wherein
the photobioreactor comprises a least a first, a second, and a third fluidically interconnected conduits, at least one of which is at least partially transparent to sunlight, the conduits together providing a flow loop enabling the liquid medium contained within the photobioreactor to flow sequentially from a region of origin within the flow loop through the first, second, and third conduits and back to the region of origin, wherein
the first, second, and third conduits are constructed and arranged so that at least one of the conduits forms an angle, with respect to the horizontal, that differs from an angle formed with respect to the horizontal of at least one of the other conduits, and wherein
at least one of the conduits forms an angle, with respect to the horizontal, of greater than 10 degrees and less than 90 degrees.

31. A method as in claim 19, wherein the establishing act comprises:
introducing a first stream of a gas to be treated by the photobioreactor to a first gas sparger configured and positioned to introduce the gas stream into a first conduit of the photobioreactor;
introducing a second stream of the gas to be treated by the photobioreactor to a second gas sparger configured and positioned to introduce the gas stream into a second conduit of the photobioreactor;
inducing the liquid medium to flow in the first conduit in a direction that is counter-current to a direction of flow of gas bubbles formed from the first stream of gas introduced into the first conduit; and
inducing the liquid medium to flow in the second conduit in a direction that is co-current to a direction of a flow of gas bubbles formed from the second stream of gas introduced into the second conduit.

32. A method as in claim 14, wherein the at least one species of photosynthetic organisms within the photobioreactor comprises algae.

33. A method of producing hydrogen comprising acts of:
providing a liquid medium comprising at least one species of photosynthetic organisms within a photobioreactor system comprising a plurality of enclosed photobioreactor apparatuses;
exposing at least a portion of at least one of the photobioreactor apparatuses and the at least one species of photosynthetic organisms therein to sunlight as a source of light driving photosynthesis;
harvesting at least a portion of the photosynthetic organisms from a bioreactor exposed to the sunlight to form biomass; and
generating hydrogen from the biomass.

34. A hydrogen production system comprising:
a photobioreactor system comprising a plurality of enclosed photobioreactor apparatuses and containing a liquid medium therein comprising at least one species of photosynthetic organisms, at least a portion of at least one photobioreactor apparatus being configured to transmit light to the photosynthetic organisms, and the photobioreactor system comprising an inlet configured to be connectable to a source of gas to be treated and an outlet configured to release treated gas from the photobioreactor system; and
a hydrogen generating system configured to produce hydrogen gas from biomass comprising photosynthetic organisms harvested from the photobioreactor system.

35. A system as in claim 34, wherein the photobioreactor system further comprises a fluid circulator constructed and arranged to establish a flow of the liquid medium within the photobioreactor system.

36. A system as in claim 34, wherein the at least one species of photosynthetic organisms within the photobioreactor system comprises algae.

37. A system as in claim 36, further comprising a dryer configured to dry algae harvested from the photobioreactor system to form dried algal biomass.

38. A system as in claim 34, wherein the hydrogen generating system comprises at least one device configured to form a gas from the biomass through a pyrolysis process or other gasification process.

39. A system as in claim 38, wherein the hydrogen generating system further comprises a gas reactor configured to produce hydrogen gas from the gas formed by the at least one device configured to form a gas from the biomass through a pyrolysis process or other gasification process.

40. A system as in claim 34, wherein the hydrogen generating system comprises at least one bacterial fermenter configured to form hydrogen gas from the biomass.

41. A system as in claim 35, further comprising:
a computer implemented system configured to perform a simulation of liquid flow patterns within the at least one photobioreactor apparatus and, from the simulation, to calculate a first exposure interval of the photosynthetic organisms to light at an intensity sufficient to drive photosynthesis and a second exposure interval of the photosynthetic organisms to dark or light at an intensity insufficient to drive photosynthesis and to control the flow of the liquid medium within the at least one photobioreactor apparatus so as to yield a selected first exposure interval and a selected second exposure interval of the photosynthetic organisms.

42. A system as in claim 34, wherein the photobioreactor system comprises at least one gas inlet configured and positioned to introduce a stream of gas to be treated into the photobioreactor system, and wherein the photosynthetic organisms within the liquid medium, once it has been exposed to the stream of gas are able to at least partially remove from the gas CO₂ and/or NOₓ.

43. A system as in claim 42, wherein the at least one gas inlet is connected in fluid communication with a source of combustion gas derived from a power generating apparatus and/or an incinerator.

44. A system as in claim 41, wherein the selected first exposure interval and the selected second exposure interval are those yielding a desired average growth rate of the photosynthetic organisms as determined by a mathematical model that simulates the growth rate of the photosynthetic organisms when exposed to alternating periods of exposure to light at an intensity sufficient to drive photosynthesis and exposure to light at an intensity insufficient to drive photosynthesis.

45. A system as in claim 41, wherein the computer implemented system is further configured to calculate the selected first exposure interval of the photosynthetic organisms to the light at an intensity sufficient to drive photosynthesis and the selected second exposure interval of the photosynthetic organisms to dark or the light at an intensity insufficient to drive photosynthesis required to yield a desired growth rate of the photosynthetic organisms within the at least one photobioreactor apparatus, utilizing a mathematical model that simulates the growth rate of the photosynthetic organisms when exposed to alternating periods of exposure to light at an intensity sufficient to drive photosynthesis and exposure to light at an intensity insufficient to drive photosynthesis, and to establish a flow of the liquid medium within the at least one photobioreactor apparatus selected to minimize the difference between the first and second exposure intervals calculated from the simulation of liquid flow patterns and the selected first and second exposure intervals calculated from the mathematical model that simulates the growth rate of the photosynthetic organisms.

46. A system as in claim 44., further comprising
at least one sensor that is configured to monitor at least one environmental or performance condition of the photobioreactor system during operation, wherein
the computer implemented system is further configured to receive a signal from the at least one sensor.

47. A system as in claim 46, wherein the computer implemented system is further configured to utilize the at least one signal from the at least one sensor in calculating the first and second exposure intervals from the simulation of liquid flow patterns.

48. A system as in claim 47, wherein the at least one sensor is configured to monitor at least one condition selected from the group consisting of: light intensity incident on the at least one photobioreactor apparatus; optical density and/or turbidity of the liquid medium within the at least one photobioreactor apparatus; gas input flow rate to the at least one photobioreactor apparatus; liquid medium flow rate within the at least one photobioreactor apparatus; temperature of the liquid medium within the at least one photobioreactor apparatus; and temperature of a gas stream supplied to the at least one photobioreactor apparatus.

49. A system as in claim 48, wherein the computer implemented system is configured to account for changes in the at least one signal received from the at least one sensor in controlling the flow of the liquid medium within the at least one photobioreactor apparatus in essentially real-time.

50. A system as in claim 44, wherein the at least one photobioreactor apparatus comprises at least a first and a second fluidically interconnected conduits, and the photobioreactor system comprises a first gas sparger configured and positioned to introduce a gas stream into the first conduit and a second gas sparger configured and positioned to introduce a gas stream into the second conduit, and wherein
the computer implemented system is further configured to control the flow of the liquid medium within the at least one photobioreactor apparatus by controlling the overall flow rate of the gas to be treated by the at least one photobioreactor apparatus and the distribution of the overall flow rate of the gas to the first and second gas spargers.

51. A system as in claim 50, wherein the computer implemented system is further configured to control the overall flow rate of the gas and the distribution of the overall flow rate of the gas to the first and second gas spargers so as to induce a liquid flow in the first conduit having a direction that is counter-current to a direction of flow of gas bubbles in the first conduit and so as to induce a liquid flow in the second conduit having a direction that is co-current to a direction of flow of gas bubbles in the second conduit.

52. A system as in claim 35, wherein
the at least one photobioreactor apparatus comprises at least a first, a second, and a third fluidically interconnected conduits, at least one of which is at least partially transparent to light, the conduits together providing a flow loop enabling the liquid medium contained within the at least one photobioreactor apparatus to flow sequentially from a region of origin within the flow loop through the first, second, and third conduits and back to the region of origin, wherein
the first, second, and third conduits are constructed and arranged so that at least one of the conduits forms an angle, with respect to the horizontal, that differs from an angle formed with respect to the horizontal of at least one of the other conduits, and wherein
at least one of the conduits forms an angle, with respect to the horizontal, of greater than 10 degrees and less than 90 degrees.

53. A system for producing hydrogen comprising:
a photobioreactor;
means for propagating at least one species of photosynthetic organisms within the photobioreactor;
means for exposing at least a portion of the photobioreactor and the at least one species of photosynthetic organisms to sunlight as a source of light driving photosynthesis;
means for harvesting biomass comprising photosynthetic organisms from the photobioreactor; and
means for forming hydrogen gas from harvested biomass.

54. A method for facilitating the production of hydrogen comprising an act of:
providing biomass produced in an enclosed photobioreactor exposed to sunlight as a source of light driving photosynthesis.

55. A method as in claim 54, wherein the biomass is produced in a photobioreactor supplied with a feed gas comprising CO₂ and/or NOₓ, at least one of which is at least partially removed from the feed gas by at least one species of photosynthetic organism during biomass production in the photobioreactor.

56. A method as in claim 54, wherein the at least one species of photosynthetic organism comprises algae and the biomass comprises algal biomass.

57. A method of claim 54, further comprising an act of:
producing the biomass provided in the providing act.

58. A method as in claim 54, wherein the feed gas comprises combustion gas derived from a power generating apparatus and/or incinerator.

59. A method as in claim 54, further comprising an act of:
providing instructions for generating and/or directions to generate hydrogen from the biomass.

60. A method for facilitating the production of hydrogen comprising an act of:
providing biomass produced in a photobioreactor system comprising a plurality of enclosed photobioreactor apparatuses.

61. A method of producing hydrogen comprising acts of:
obtaining biomass produced in an enclosed photobioreactor exposed to sunlight as a source of light driving photosynthesis; and
generating hydrogen from the biomass.

62. A method as in claim 61, wherein the biomass is produced in a photobioreactor supplied with a feed gas comprising CO₂ and/or NOₓ, at least one of which is at least partially removed from the feed gas by at least one species of photosynthetic organism during biomass production in the photobioreactor.

63. A method as in claim 61, wherein the at least one species of photosynthetic organism comprises algae and the biomass comprises algal biomass.

64. A method as in claim 63, wherein the generating act further comprises an act of gasification or pyrolysis of the biomass.

65. A method as in claim 64, wherein the generating act further comprises reacting a gas produced by the gasification or pyrolysis act to form hydrogen gas.

66. A method as in claim 63, wherein the generating act comprises subjecting the biomass to bacterial digestion to form hydrogen gas.

67. A method of producing hydrogen comprising acts of:
obtaining biomass produced in a photobioreactor system comprising a plurality of enclosed photobioreactor apparatuses; and
generating hydrogen from the biomass.

68. An integrated combustion and hydrogen production method comprising acts of:
burning a fuel with a combustion device to produce a combustion gas stream;
passing the combustion gas to an inlet of an enclosed photobioreactor containing a liquid medium therein comprising at least one species of photosynthetic organisms and exposed to sunlight as a source of light driving photosynthesis;
at least partially removing at least one substance from the combustion gas with the photosynthetic organisms, the at least one substance being utilized by the organisms for growth and reproduction;
removing at least a portion of the at least one species of photosynthetic organisms from the photobioreactor to form a biomass product; and
using at least a portion of the biomass product to produce hydrogen gas.

69. An integrated combustion and hydrogen production method as in claim 68, further comprising an act of:
feeding the combustion gas stream to a dryer and cooling the combustion gas stream in the dryer, thereby forming a cooled combustion gas that comprises the combustion gas passed to the inlet of the photobioreactor in the passing act.

70. An integrated combustion and hydrogen production method as in claim 69, the removing act comprises:
removing at least a portion of the liquid medium comprising the at least one species of photosynthetic organisms from the photobioreactor; and
drying the liquid medium with the dryer fed with the combustion gas in the feeding act to produce a dried biomass product.

71. An integrated combustion and hydrogen production method as in claim 70, further comprising an act of:
using at least a portion of the dried biomass product as the fuel and/or to produce the fuel burned in the burning act.

72. An integrated combustion method and hydrogen production method as in claim 68, wherein the combustion device comprises or forms part of an electricity generating and/or incineration facility.

73. An integrated combustion method and hydrogen production method as in claim 68, wherein the at least one substance at least partially removed from the combustion gas in the at least partially removing act comprises CO₂ and/or NO_{X}.

74. An integrated combustion method and hydrogen production method as in claim 68, further comprising, after the at least partially removing act, an act of:
releasing treated gas from a gas outlet of the photobioreactor.

75. An integrated combustion method and hydrogen production method as claim 74, wherein the treated gas is released from the gas outlet of the photobioreactor and directed, directly or indirectly, to an inlet of a smoke stack.

76. An integrated combustion method and hydrogen production method as in claim 68, wherein the at least one species of photosynthetic organisms comprises algae and wherein the dried biomass product comprises a dried algal biomass product.

77. An integrated combustion method and hydrogen production method as in claim 76, wherein the dried algal biomass product is used to produce at least one fuel product comprising an oil and/or a combustible organic gas.

78. An integrated combustion method and hydrogen production method as in claim 68, wherein the using act comprises:
producing an organic gas from the biomass product through pyrolysis or gasification of the biomass product.

79. An integrated combustion method and hydrogen production method as in claim 78, wherein the using act further comprises:
reacting the organic gas with water to produce a product gas comprising hydrogen gas.

80. An integrated combustion method and hydrogen production method as in claim 78, wherein the using act further comprises:
separating the hydrogen gas from other gases in the product gas to produce purified hydrogen gas and at least one by-product gas.

81. An integrated combustion method and hydrogen production method as in claim 80, wherein the at least one by-product gas comprises CO₂.

82. An integrated combustion method and hydrogen production method as in claim 81, wherein the at least one by-product gas further comprises NOₓ.

83. An integrated combustion method and hydrogen production method as in claim 81, further comprising an act of:
passing at least a portion of the at least one by-product gas to the inlet of the photobioreactor.

82. An integrated combustion method and hydrogen production method as in claim 82, further comprising an act of:
passing at least a portion of the at least one by-product gas to the inlet of the photobioreactor.

85. An integrated combustion method and hydrogen production method as in claim 68, wherein the using act comprises:
producing a product gas comprising hydrogen gas from the biomass product through bacterial fermentation of the biomass product.

86. An integrated combustion method and hydrogen production method as in claim 85, wherein the using act further comprises:
separating the hydrogen gas from other gases in the product gas to produce purified hydrogen gas and at least one by-product gas.

87. An integrated combustion method and hydrogen production method as in claim 86, wherein the at least one by-product gas comprises CO₂.

88. An integrated combustion method and hydrogen production method as in claim 87, further comprising an act of:
passing at least a portion of the at least one by-product gas to the inlet of the photobioreactor.

89. An integrated combustion and hydrogen production method comprising acts of:
burning a fuel with a combustion device to produce a combustion gas stream;
passing the combustion gas to an inlet of a photobioreactor system comprising a plurality of enclosed photobioreactor apparatuses and containing a liquid medium therein comprising at least one species of photosynthetic organisms;
at least partially removing at least one substance from the combustion gas with the photosynthetic organisms, the at least one substance being utilized by the organisms for growth and reproduction;
removing at least a portion of the at least one species of photosynthetic organisms from the photobioreactor system to form a biomass product; and
using at least a portion of the biomass product to produce hydrogen gas.
